(19) United States
(12) Patent Application Publication
(10) Pub. No.: US 2026/0018258 A1
(43) Pub. Date: Jan. 15, 2026

(54) SYSTEM AND METHOD FOR ESTIMATING PERMEABILITY OF A BIOTURBATED RESERVOIR

(71) Applicants: KING FAHD UNIVERSITY OF PETROLEUM AND MINERALS, Dhahran (SA); University of Kansas, Lawrence, KS (US)

(72) Inventors: Hassan Abdalla Eltom MOSAD, Dhahran (SA); Robert H. GOLDSTEIN, Lawrence, KS (US)

(73) Assignees: KING FAHD UNIVERSITY OF PETROLEUM AND MINERALS, Dhahran (SA); University of Kansas, Lawrence, KS (US)

(21) Appl. No.: 18/975,822

(22) Filed: Dec. 10, 2024

Related U.S. Application Data
(60) Provisional application No. 63/670,523, filed on Jul. 12, 2024.

(51) Int. Cl. G16C 60/00; G01N 15/08

(52) U.S. Cl. CPC G16C 60/00; G01N 15/08

(57) ABSTRACT

A method of estimating a permeability of a bioturbated reservoir based on a Thalassinoides connectivity. The method includes generating geocellular models from a Thalassinoides morphology, converting the geocellular models to training images each having a host rock matrix and Thalassinoides burrows. The method further includes measuring statistical parameters from the training images to obtain a width of a Thalassinoides shaft, creating samples each having a burrow percentage, a burrow size, and a sample cross section from the geocellular models. Further, determining a largest connected burrow volume (LCBV) of each sample based on the Thalassinoides burrows to obtain a burrow connectivity and computing the Thalassinoides connectivity based on the burrow connectivity to thereby estimate the permeability of the bioturbated reservoir.

### CROSS REFERENCE TO RELATED APPLICATION

The present application claims benefit of priority to U.S. Provisional Application No. 63/670,523 having a filing date of Jul. 12, 2024 which is incorporated herein by reference in its entirety.

### STATEMENT REGARDING PRIOR DISCLOSURE BY THE INVENTORS

Aspects of the present disclosure are described in “Digital rock modeling to quantify scale dependence of petrophysical measurements in burrowed reservoir rocks: An example using Thalassinoides”, published in Marine and Petroleum Geology, Volume 155, 106412, which is incorporated herein by reference in its entirety.

### STATEMENT OF ACKNOWLEDGEMENT

Support provided by the College of Petroleum Engineering and Geosciences in King Fahd University of Petroleum and Minerals, Saudi Arabia, under research startup grant SF19031 is gratefully acknowledged.

### BACKGROUND

### Technical Field

The present disclosure is directed towards digital rock modelling, and more particularly, directed towards a system and a method for estimating a permeability of a bioturbated reservoir.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention. Permeability of subsurface carbonate reservoirs is an important parameter for formation evaluation and fluid flow simulation. Yet, permeability cannot be measured directly from rocks of subsurface reservoirs using conventional well logs. However, permeability may be inferred from well testing and production or may be modelled, with varying degrees of success, from well logs such as porosity and NMR logs. Reservoir permeability has been estimated through laboratory measurements from cored reservoir rocks. Such laboratory measurements have been used to calibrate well logs to calculate a permeability log that is eventually used in fluid flow simulation.

The rock samples used for such laboratory measurements have core plugs diameters in the range of 1 inch to 1.5 inch and conventional reservoir core diameter of about 4 inch. These diameters are small compared to the actual scale of the pore systems in many carbonate reservoirs. In some cases, the dimensions of the samples analyzed are not large enough to capture the dimensions that include the representative elementary volume (REV). REV refers to the smallest volume of a sample with correct dimensions from which a measured parameter is size-independent. Measured permeabilities on such small samples are unlikely to represent the actual permeability in reservoirs, because of the large scale of the heterogeneity in pore system of the reservoir. It has been demonstrated that use of such permeability measurements leads to errors when predicting reservoir performance. One source of such large-scale permeability heterogeneity is induced by burrows in carbonate reservoir rocks. In carbonate strata, burrows may enhance the porosity and permeability of a tight rock matrix, if the burrows remain open or are filled with sediment with porosity and permeability higher than the matrix. An example is carbonate reservoir with Thalassinoides, which refers to a branched burrow with a boxwork pattern and horizontal, as well as vertical penetration. In many carbonate reservoirs, Thalassinoides infills may be dolomitized and host intercrystalline porosity, making them considerably more porous and permeable than a surrounding limestone matrix. In some carbonate reservoirs, Thalassinoides are infilled with grain-dominated sediments that include interparticle porosity with substantially more porosity and permeability relative to surrounding muddy carbonate. Further, Thalassinoides can be open or partially open, when this occurs, the burrow porosity of Thalassinoides may result in flow zones that are extremely permeable. In cases where Thalassinoides enhances the porosity and permeability of carbonate reservoirs, burrow connectivity controls the permeability, because the connected Thalassinoides network provides permeability passageways in otherwise less permeable host rock matrix. Capturing the full connectivity of Thalassinoides networks is not guaranteed. Small sample sizes may, or may not, be suitable to represent the permeability of Thalassinoides bearing carbonate reservoirs. Determining what minimum sample size is needed to represent permeability and porosity requires systematically analyzing a variety of burrow sizes, burrow percentages, and sample dimensions. Getting such a variety from natural samples may be highly challenging. Therefore, in order to overcome above stated challenges, a need arise for an efficient and accurate digital modeling approach as an efficient alternative.

Hence, it is one object of the present disclosure to provide a system for estimating a permeability of a bioturbated reservoir and a method thereof, that may circumvent the aforementioned drawbacks.

### SUMMARY

In an exemplary embodiment, a method of estimating a permeability of a bioturbated reservoir based on a Thalassinoides connectivity is described. The method includes generating a plurality of geocellular models from a Thalassinoides morphology, converting the plurality of geocellular models to a plurality of training images each having a host rock matrix and Thalassinoides burrows. The method further includes measuring a plurality of statistical parameters from the plurality of training images to obtain a width of a Thalassinoides shaft, creating a plurality of samples each having a burrow percentage, a burrow size, and a sample cross section from the plurality of geocellular models. Further, the method includes determining a largest connected burrow volume (LCBV) of each sample of the plurality of samples based on the Thalassinoides burrows to obtain a burrow connectivity and computing the Thalassinoides connectivity based on the burrow connectivity to thereby estimate the permeability of the bioturbated reservoir.

In some embodiments, each geocellular model of the plurality of geocellular models includes a three-dimensional multipoint statistics (3DMPS) model having a three-dimensional volume of about 1 m³.

In some embodiments, the plurality of geocellular models includes 18 3DMPS models.

In some embodiments, each 3DMPS model of the 18 3DMPS models is constructed based on an Eltom method.

In some embodiments, the creating further includes extracting a plurality of columnar samples from each geocellular model of the plurality of geocellular models, extracting a plurality of subsamples each having a column cross section from each columnar sample of the plurality of columnar samples, and combining the plurality of subsamples to obtain the plurality of samples. An area of the column cross section is between 25 cm² and 900 cm².

In some embodiments, the plurality of subsamples includes 6 subsamples and wherein the area of the column cross section of the plurality of subsamples is 25 cm², 100 cm², 225 cm², 400 cm², 625 cm², or 900 cm².

In some embodiments, the burrow percentage of each sample of the plurality of samples is selected from the group consisting of 20%, 50%, and 75%.

In some embodiments, the burrow size of each sample of the plurality of samples is between 2.6 cm and 9 cm.

In some embodiments, the determining further includes determining the LCBV of each sample of the plurality of samples based on an Eltom method, determining whether the LCBV of each sample of the plurality of samples is connected across from a top to a bottom of each sample of the plurality of samples, indicating, when the LCBV is connected across from the top to the bottom, the LCBV as a connected burrow, and measuring a length and a position of the LCBV to determine the burrow connectivity.

In some embodiments, the computing further includes dividing the plurality of samples into a training set and a validation set, running a logistic regression analysis with the training set and the burrow connectivity to obtain a logistic regression result. The computing further includes validating the logistic regression result with the validation set and the burrow connectivity to obtain a probability equation, and computing the Thalassinoides connectivity based on the probability equation.

In another exemplary embodiment, a system for estimating a permeability of bioturbated reservoirs represented by a Thalassinoides connectivity is described. The system includes a processor configured to execute a program instruction, and a memory having the program instruction, where the memory is connected to the processor. The system further includes an input device connected to the processor and configured to receive a plurality of computed tomography (CT) scan images each having a Thalassinoides morphology, and a display device configured to display the Thalassinoides connectivity. The program instruction includes generating a plurality of geocellular models from the Thalassinoides morphology of the plurality of CT scan images. The program instruction further includes converting the plurality of geocellular models to a plurality of training images each having a host rock matrix and Thalassinoides burrows, measuring a plurality of statistical parameters from the plurality of training images to obtain a width of a Thalassinoides shaft, creating a plurality of samples each having a burrow percentage, a burrow size, and a sample cross section from the plurality of geocellular models. The program instruction further determines a largest connected burrow volume (LCBV) of each sample of the plurality of samples based on the Thalassinoides burrows to obtain a burrow connectivity, and computes the Thalassinoides connectivity based on the burrow connectivity to thereby estimate the permeability of bioturbated reservoirs.

In some embodiments, each geocellular model of the plurality of geocellular models includes a three-dimensional multipoint statistics (3DMPS) model having a three-dimensional volume of about 1 m³.

In some embodiments, the plurality of geocellular models includes 18 3DMPS models.

In some embodiments, each 3DMPS model of the 18 3DMPS models is constructed based on an Eltom method.

In some embodiments, the creating further includes extracting a plurality of columnar samples from each geocellular model of the plurality of geocellular models, extracting a plurality of subsamples each having a column cross section from each columnar sample of the plurality of columnar samples, and combining the plurality of subsamples to obtain the plurality of samples. An area of the column cross section is between 25 cm² and 900 cm².

In some embodiments, the plurality of subsample includes 6 subsamples and where the area of the column cross section of the plurality of subsamples is 25 cm², 100 cm², 225 cm², 400 cm², 625 cm², or 900 cm².

In some embodiments, the burrow percentage of each sample of the plurality of samples is selected from the group consisting of 20%, 50%, and 75%.

In some embodiments, the burrow size of each sample of the plurality of samples is between 2.6 cm and 9 cm.

In some embodiments, the determining further includes determining the LCBV of each sample of the plurality of samples based on an Eltom method, determining whether the LCBV of each sample of the plurality of samples is connected across from a top to a bottom of each sample of the plurality of samples, indicating, when the LCBV is connected across from the top to the bottom, the LCBV as a connected burrow, and measuring a length and a position of the LCBV to determine the burrow connectivity.

In some embodiments, the computing further includes dividing the plurality of samples into a training set and a validation set, running a logistic regression analysis with the training set and the burrow connectivity to obtain a logistic regression result, validating the logistic regression result with the validation set and the burrow connectivity to obtain a probability equation, and computing the Thalassinoides connectivity based on the probability equation.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic block diagram of a system configured to execute a set of program instruction and thereby to estimate a permeability of bioturbated reservoirs, according to certain embodiments;

FIG. 2 is a schematic flow chart of a method for estimating the permeability of the bioturbated reservoir represented by a Thalassinoides connectivity, according to certain embodiments;

FIG. 3A is a schematic block diagram depicting generation of a plurality of digital samples (540 samples) of Thalassinoides using multipoint statistical modelling (MPS), according to certain embodiments;

FIG. 3B is a schematic diagram depicting use of the plurality of digital samples in generating a probability equation for Thalassinoides connectivity in a laboratory setting, according to certain embodiments;

FIG. 3C is a schematic diagram depicting an implementation of the plurality of digital samples and the probability equation in order to predict Thalassinoides connectivity in a laboratory setting, according to certain embodiments;

FIG. 4A is a schematic perspective illustration of a digital three-dimensional (3D) cube representing 1 cubic meter (m³) MPS model showing sampling locations in 3D, according to certain embodiments;

FIG. 4B is a schematic illustration depicting an upper surface of the digital sample showing six cross sections of the digital samples, according to certain embodiments;

FIG. 4C is a schematic perspective illustration of a set of exemplary digital samples (six samples) at one particular sampling location with one particular burrow percentage (BP) and burrow size (BS), according to certain embodiments;

FIG. 5A is a schematic perspective illustration of exemplary results of MPS modelling on digital samples with 1 m³ volume and a BS of a 9.0 cm, according to certain embodiments;

FIG. 5B is a schematic perspective illustration of exemplary results of MPS modelling on digital samples with 1 m³ volume and a BS of a 5.1 cm, according to certain embodiments;

FIG. 5C is a schematic perspective illustration of exemplary results of MPS modelling on digital samples with 1 m³ volume and a BS of a 4.0 cm, according to certain embodiments;

FIG. 5D is a schematic perspective illustration of exemplary results of MPS modelling on digital samples with 1 m³ volume and a BS of a 3.4 cm, according to certain embodiments;

FIG. 5E is a schematic perspective illustration of exemplary results of MPS modelling on digital samples with 1 m³ volume and a BS of a 2.7 cm, according to certain embodiments;

FIG. 5F is a schematic perspective illustration of exemplary results of MPS modelling on digital samples with 1 m³ volume and a BS of a 2.6 cm, according to certain embodiments;

FIG. 6A is a graph depicting histogram of a diameter of the modelled Thalassinoides in a first 3D model having a cell number of 100³, according to certain embodiments;

FIG. 6B is a graph depicting histogram of a diameter of the modelled Thalassinoides in a second 3D model having a cell number of 200³, according to certain embodiments;

FIG. 6C is a graph depicting histogram of a diameter of the modelled Thalassinoides in a third 3D model having a cell number of 300³, according to certain embodiments;

FIG. 6D is a graph depicting histogram of a diameter of the modelled Thalassinoides in a fourth 3D model having a cell number of 400³, according to certain embodiments;

FIG. 6E is a graph depicting histogram of a diameter of the modelled Thalassinoides in a fifth 3D model having a cell number of 500³, according to certain embodiments;

FIG. 6F is a graph depicting histogram of a diameter of the modelled Thalassinoides in a sixth 3D model having a cell number of 600³, according to certain embodiments;

FIG. 7A is a graph depicting cross-plots between sample cross section (SCS) and probability of Thalassinoides connectivity displayed based on the BP for a BS of 9.0 cm, according to certain embodiments;

FIG. 7B is a graph depicting cross-plots between sample cross section and probability of Thalassinoides connectivity displayed based on the BP for a BS of 5.1 cm, according to certain embodiments;

FIG. 7C is a graph depicting cross-plots between sample cross section and probability of Thalassinoides connectivity displayed based on the BP for a BS of 4.0 cm, according to certain embodiments;

FIG. 7D is a graph depicting cross-plots between sample cross section and probability of Thalassinoides connectivity displayed based on the BP for a BS of 3.4 cm, according to certain embodiments;

FIG. 7E is a graph depicting cross-plots between sample cross section and probability of Thalassinoides connectivity displayed based on the BP for a BS of 2.7 cm, according to certain embodiments;

FIG. 7F is a graph depicting cross-plots between sample cross section and probability of Thalassinoides connectivity displayed based on the BP for a BS of 2.6 cm, according to certain embodiments;

FIG. 8A is a graph depicting exceedance probability trend with respect to length of the largest connected burrow volume (LCBV) in the plurality of digital samples based on BP, according to certain embodiments;

FIG. 8B is a graph depicting exceedance probability trend with respect to length of the LCBV in the plurality of digital samples based on BS, according to certain embodiments;

FIG. 8C is a graph depicting exceedance probability trend with respect to length of the LCBV in the plurality of digital samples based on SCS, according to certain embodiments;

FIG. 9A is a graph depicting a result of binary logistic regression with standardized coefficients of independent variables (BS, BP, and SCS), according to certain embodiments;

FIG. 9B is a graph depicting receiver operating characteristics (ROC) for internal validation of the data, according to certain embodiments;

FIG. 9C is a graph depicting ROC for external validation using 108 digital samples, according to certain embodiments;

FIG. 10 is a cross plot between sample cross section and probability of Thalassinoides connectivity calculated from the equation of the logistic regression model, according to certain

FIG. 11A is a graph depicting histogram and cumulative probability curve for pLCBV in the plurality of digital samples with 25 cm² cross section and a length of the sample being 1 cm, according to certain embodiments;

FIG. 11B is a graph depicting histogram and cumulative probability curve for pLCBV in the plurality of digital samples with 25 cm² cross section and a length of the sample being 2.5 cm, according to certain embodiments;

FIG. 11C is a graph depicting histogram and cumulative probability curve for pLCBV in the plurality of digital samples with 25 cm² cross section and a length of the sample being 5.0 cm, according to certain embodiments;

FIG. 12A is a graph depicting histogram and cumulative probability curve for the pLCBV in the plurality of digital samples with 100 cm² cross section and a sample length of 10 cm, according to certain embodiments;

FIG. 12B is a graph depicting histogram and cumulative probability curve for the pLCBV in the plurality of digital samples with 100 cm² cross section and a sample length of 20 cm, according to certain embodiments; and

FIG. 12C is a graph depicting histogram and cumulative probability curve for the pLCBV in the plurality of digital samples with 100 cm² cross section and a sample length of 25 cm, according to certain embodiments.

FIG. 13 is an illustration of a non-limiting example of details of computing hardware used in the system of FIG. 1, according to certain embodiments;

FIG. 14 is an exemplary schematic diagram of a data processing system used within the system, according to certain embodiments;

FIG. 15 is an exemplary schematic diagram of a processor used with the system, according to certain embodiments; and

FIG. 16 is an illustration of a non-limiting example of distributed components which may share processing with a controller, according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed towards a system and a method for estimating permeability of bioturbated reservoirs represented by a Thalassinoides connectivity. Thalassinoides is a trace fossil including branching burrows in sedimentary rock, attributed to ancient marine or freshwater crustaceans known as ghost shrimp. The burrows often display characteristics of Y-shaped and U-shaped branching pattern, indicating an activity of crustaceans as they excavated through sedimentary rock formation. Since burrow connectivity have shown that burrow morphology, burrow abundance, and burrow size are factors impacting a sampling scheme required for representing permeability of bioturbated reservoir, the present disclosure discloses use of multipoint statistics modelling and applies it to Thalassinoides. The present disclosure aims to simulate a range of burrow abundances and sizes, and subsequently interrogate the outcome to develop a statistical model that allows sampling strategies to be designed.

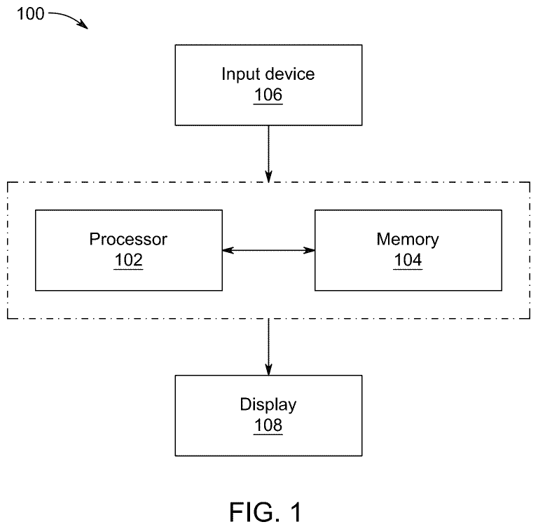

Referring to FIG. 1, a schematic block diagram of a computing environment including a system 100 is illustrated, according to certain embodiments. In particular, the system 100 is designed for estimating a permeability of bioturbated reservoirs represented by a Thalassinoides connectivity. In general, bioturbated reservoirs refer to geological formations where the sedimentary layers have been extensively reworked by physical activity of organisms. The bioturbation process may alter the original bedding and sediment structures, leading to a more homogenized and often complex arrangement of grains and particles of the geological formations. The activity of such organisms, typically invertebrates like worms and bivalves, can significantly impact properties of the reservoirs, such as porosity and permeability, which are crucial for the storage and flow of fluids like oil, gas, and water. In some embodiments, the system 100 includes a processor 102 and a memory 104. The processor 102 is configured to execute program instruction stored in the memory 104. In other words, the memory 104 having program instruction is connected to the processer 102 electronically. In some embodiments, an input device 106 is connected to the processor 102 and the input device 106 is configured to receive a plurality of computed tomography (CT) scan images. Further, each CT scan image has a Thalassinoides morphology. In an embodiment, the program instruction, as stored in the memory 104, is a set of digital instructions which includes generating a plurality of geocellular models from the Thalassinoides morphology of the plurality of CT scan images received by the input device 106. In particular, a large diameter core of a particular bioturbated reservoir underwent CT scans in order to generate the plurality of CT scan images. The processor 102 executes the program instructions to create a high-resolution geocellular model. In some embodiments, each of the plurality of geocellular models includes a three-dimensional multipoint statistics (3DMPS) model having a three-dimensional volume of about 1 cubic meter (m³). In general, a geocellular model is a three-dimensional representation of a geological space of bioturbated reservoirs, used for characterization and simulation. Geocellular models incorporates a plurality of geological, geophysical, and petrophysical data to create a detailed depiction of subsurface structures. The geocellular model includes cellular grids that align with major structural components like faults and horizons, allowing for greater accuracy in simulating fluid flow and reservoir potential. Geocellular models are crucial for making informed decisions in field development and operational planning, as they help understand how complex reservoir attributes influence fluid dynamics. Hence, the geocellular models created by the processor 102 upon execution of program instructions, and specifically, upon processing CT scan images allows for greater accuracy in determining the permeability of the bioturbated reservoir. Further, the plurality of geocellular models includes 18 3DMPS models. In an aspect, each 3DMPS model of the 18 3DMPS models is constructed based on an Eltom method. The “Eltom method” refers to creating 3DMPS models having three variables: burrow morphology (three end-member burrow morphologies boxwork, vertical and horizontal), bioturbation intensity expressed as burrow volume percentage (25 intensities, ranging from 2% to 50%, for example), and matrix properties held consistent for all models. These variables were simulated in the high-resolution geocellular models (e.g. 1 m³ model having 8×10⁶ cells, resulting in 75 models).

In some embodiments, the program instruction is encoded in such a way that they convert the plurality of geocellular models to a plurality of training images, each image having a host rock matric and Thalassinoides burrows. In some embodiments, the program instruction further includes measuring a plurality of statistical parameters from the plurality of training images to obtain a width of a Thalassinoides shaft. The program instruction creates a plurality of samples, each have a burrow percentage (BP), a burrow size (BS), and a sample cross section from the plurality of geocellular models. In other words, the plurality of training images is used to generate modelled Thalassinoides with a range of bioturbation intensity expressed as the BP and the BS. In an example, six Thalassinoides burrow sizes varying from 2.6 cm to 9 cm were modelled by varying a cell size of the 1 m³ 3D volume of the plurality of geocellular models. In an embodiment, creating the plurality of samples further includes extracting a plurality of columnar samples from each geocellular model of the plurality of geocellular models. In addition, creating the plurality of samples further includes extracting a plurality of subsamples each having a column cross section from each columnar sample of the plurality of columnar samples, where an area of the column cross section is between 25 cm² and 900 cm². In an example of the present disclosure, each 3DMPS model with a single BP and BS was extracted into 30 columnar samples, thus a total of 18 3DMPS model (as stated above) were converted into 540 columnar samples to evaluate a range of BP, BS, and sample cross section. The BP of each sample of the plurality of samples is selected from the group consisting of 20%, 50%, and 75%. As described above, the BS of each sample of the plurality of samples is between 2.6 cm and 9 cm. In other words, the plurality of subsamples was combined in order to obtain the plurality of subsamples. Examination of the 540 samples may help in determining the cross-sectional area needed to represent permeability of the reservoir, based on which the samples were modelled. Further, the plurality of subsamples includes 6 subsamples. The area of the column cross section of the plurality of subsamples is 25 cm², 225 cm², 400 cm², 625 cm², or 900 cm². In an example, the burrow connectivity (permeability) was analysed in a computing software provided by Petrel™.

The program instruction stored in the memory 104 of the system 100 further includes determining a largest connected burrow volume (LCBV) of each sample of the plurality of samples. The LCBV is determined based on the Thalassinoides burrows to obtain a burrow connectivity. In some embodiments, determining the LCBV further includes determining the LCBV of each sample of the plurality of samples based on the Eltom method. The program instruction stored in the memory 104 determines whether the LCBV of each sample of the plurality of samples is connected across from a top to a bottom of each sample of the plurality of samples, and further indicates, when the LCBV is connected across from the top to the bottom, the LCBV as a connected burrow. In other words, the LCBV may be considered a proxy for burrow connectivity, as such, if the LCBV touches an upper face and a lower face of the plurality of samples (top and bottom of each sample), then the 3DMPS model has vertical connected burrow and subsequently the bioturbated reservoirs have high permeability. In addition, the program instruction measures a length and a position of the LCBV to determine the burrow connectivity. Furthermore, the program instruction stored in the memory 104 includes computing the Thalassinoides connectivity based on the burrow connectivity to thereby estimate the permeability of the bioturbated reservoirs. The computing further includes dividing the plurality of samples into a training set and a validation set. In an example, each of the 540 samples were randomly divided into two subsets (training set and validation set). A total of 432 samples were used as the training set, running a logistic regression analysis with the training set and the burrow connectivity to obtain a logistic regression result for regression modelling. Further, a total of 108 samples out of the 540 samples were used as external validation samples, validating the logistic regression result with the validation set and the burrow connectivity to obtain a probability equation. The validation is conducted by comparing a predicted binary class to their actual class, of the Thalassinoides connectivity against an isolated burrow volume from the logistic regression model. Such comparison may assist in evaluating sensitivity and specificity of the logistic regression results. In some embodiments, the Thalassinoides connectivity is computed based on the aforementioned probability equation. A mathematical model of the probability equation and other specific details of logistic regression modelling are provided in the subsequent paragraph(s) in the ‘examples’ section.

Further, the system 100 includes a display device 108 configured to display the Thalassinoides connectivity. In particular, the display device 108 may refer to a graphic display, a digital display, a touch screen display, a dot-matrix display, a LED display, an LCD display, or a combination thereof. In some embodiments, the display device 108 is configured to receive a set of data from the processor 102 to display the set of data by converting it into a readable medium.

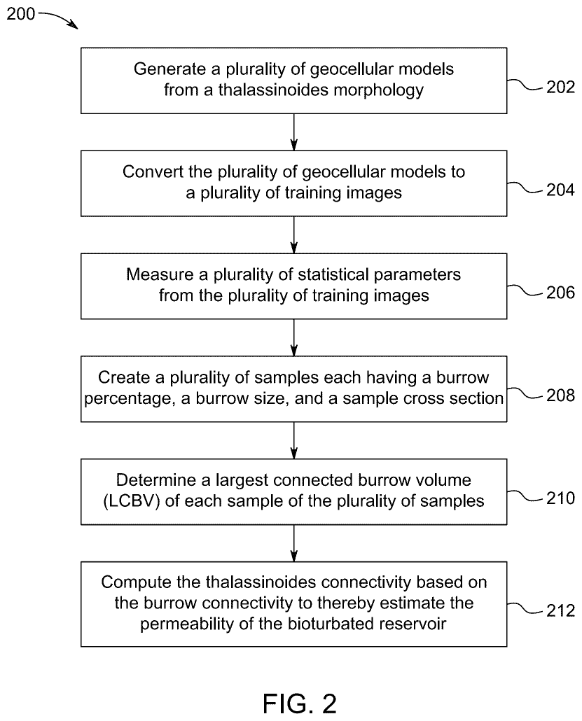

Referring to FIG. 2, a schematic flow chart of a method 200 for estimating the permeability of the bioturbated reservoir based on the Thalassinoides connectivity is illustrated, according to certain embodiments. The order in which the method 200 is described is not intended to be construed as a limitation, and any number of the described method steps can be combined in any order to implement the method 200. Additionally, individual steps may be removed or skipped from the method 200 without departing from the spirit and scope of the present disclosure. In particular, the method 200 describes a flow and order of implementation of the processes included in the processor 102 of the system 100. The method steps highlight major processes involved in the estimation of the permeability of bioturbated reservoirs. More detailed explanation of implementation of the method steps is provided in the ‘examples’ section of the present disclosure for sake of brevity in explanation.

At a step 202, the method 200 includes generating the plurality of geocellular models from the Thalassinoides morphology. As described with respect to the system 100, each geocellular model of the plurality of geocellular models includes the 3DMPS model having 3D volume of about 1 m³, further, other details pertaining to the plurality of models generated at the step 202 remains similar to the plurality of geocellular models of the system 100. The step 202 details the process of generating the plurality of geocellular models, this helps in regard to estimating the permeability of the bioturbated reservoirs more accurately. Further, the plurality of geocellular models may be used to train the system 100 in order to produce consistent results.

At a step 204, the method 200 includes converting the plurality of geocellular models to a plurality of training images each having a host rock matrix and Thalassinoides burrows. The host rock matrix and the Thalassinoides burrows are collectively known as rock fabrics. The rock fabrics and associated parameters are crucial for generation of accurate digital models of the Thalassinoides with a range of bioturbation intensity. The training images of step 204 may further be used to produce algorithms for sample dimensions that may represent the permeability of a particular reservoir correctly. Thus, the plurality of geocellular models are modelled with high accuracy and meticulous attention to detail in order to produce the training images with high accuracy.

At step 206, the method 200 includes measuring a plurality of statistical parameters from the plurality of training images generated at step 204, to obtain a width of a Thalassinoides shaft. The Thalassinoides shafts and their corresponding sizes may indicate a source of the Thalassinoides. Further, in an example, JMicroVision open source software was used for the analysis of the plurality of images generated at step 204, for accurate measurement of the Thalassinoides shaft at step 206. In some aspects, the Thalassinoides shaft and corresponding dimensions may be used as a proxy for burrow size. Furthermore, statistical parameters such as, mean, median, minima, maxima, and standard deviation may be used to conduct the measuring of the width of the Thalassinoides shaft as described in step 206.

At step 208, the method 200 includes creating the plurality of samples each having the BP, the BS, and the sample cross section from the plurality of geocellular models. At step 208, the method 200 further includes extracting the plurality of columnar samples from each geocellular model of the plurality of models. The column cross section of the plurality of columnar samples is between 25 cm² to 900 cm². As such, 540 columnar samples are analyzed to obtain the burrow connectivity. Further, at step 210, the method 200 includes determining the LCBV of each sample of the plurality of samples based on the Thalassinoides burrows to obtain the burrow connectivity. In some aspects, the BP, the BS, and the sample cross section are parameters that may be used to determine the LCBV. The LCBV may be determined from top of the sample to the bottom of the sample, or, from one side of the sample to another side of the sample. If the LCBV connected the top of the sample to the bottom of the sample, then the sample may be marked as permeable, and further may percolate a fluid from the top to the bottom of the sample. In case the LCBV does not touch the top and the bottom surfaces of the sample, then the burrow volumes may not provide permeability pathways from the bottom to the top of a measured sample.

At step 212, the method 200 includes computing the Thalassinoides connectivity based on the burrow connectivity to thereby estimate the permeability of the bioturbated reservoir. The method 200 further includes dividing the plurality of samples into training set and validation set. The logistic regression modelling, as described above with respect to system 100 is then applied in order to generate a probability equation to determine the permeability of bioturbated reservoirs. Further, the burrow connectivity approach based on the LCBV may be applied to any trace fossil morphology. A machine learning model may further be trained on the basis of the method 200 and subsequent steps 202 to 212. The machine learning model may determine patterns of relationship between the BP, the BS, sample cross section, sample length, burrow morphology, and burrow connectivity.

### EXAMPLES

The following examples demonstrate the system 100 for estimating the permeability of the bioturbated reservoir based on the Thalassinoides connectivity, and the method 200 thereof. The examples are provided solely for illustration and are not to be construed as limitations of the present disclosure, as many variations thereof are possible without departing from the spirit and scope of the present disclosure.

### Example 1: Multi-Point Statistics (MPS) Models

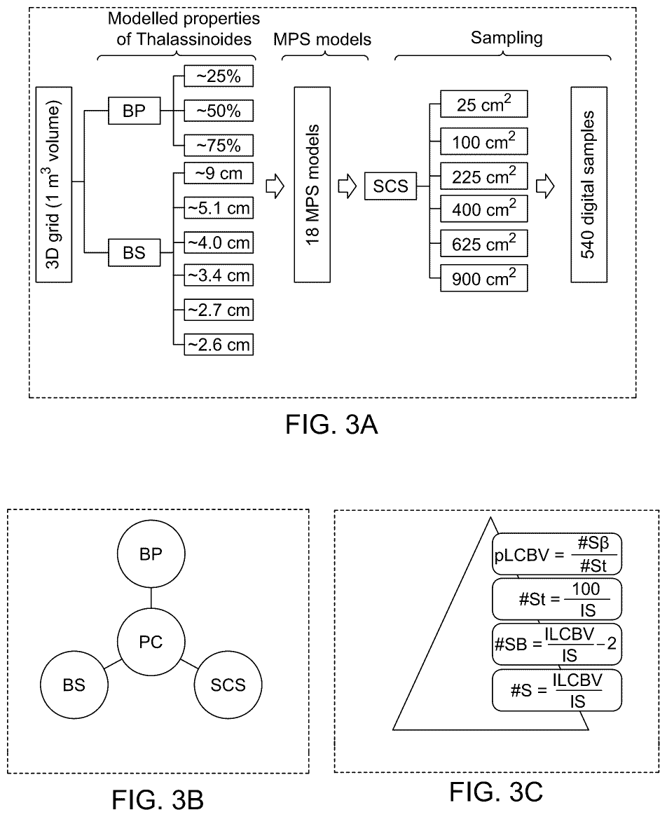

In accordance with the present disclosure, 18 MPS three-dimensional (3D) simulation scenarios of Thalassinoides were modelled in a 3D grid with a volume of about 1 cubic meter (m³), as shown in FIG. 3A. The MPS models were constructed in Petrel™ 2020 using the methods described by Eltom and coworkers in various studies from 2019 to 2021. The workflow starts with capturing Thalassinoides morphology in a high-resolution geocellular model with a 3D volume of about 1 m³ and converting the geocellular model to a training image. The training image includes two rock fabrics: the host rock matrix, and the Thalassinoides burrows. The training image is used to generate modelled Thalassinoides with a range of bioturbation intensity, expressed as burrow percentage (BP) and burrow size (BS), as shown in FIG. 3A. Each one of the 18 scenarios of Thalassinoides was modelled in a 1 m³ 3D volume representing three BPs of about 25%, 50%, and 75%. The above specified BPs were selected as previous experimentation had indicated these BPs as a likely range of BP covering natural systems, yet still, with 100% probability, that burrows connect across the sides of the 1 m³ 3D volume. This allows for evaluation of the sampling that is necessary to reflect connectivity. In addition, six Thalassinoides burrow sizes (expressed as the width of the Thalassinoides shafts), from 2.6 cm to 9 cm, were modelled by varying the cell size of the 1 m³ 3D volume. In an example, the lower limit of the size range, 2.6 cm, may represent the Thalassinoides shafts of Jubaila in Saudi Arabia, whereas the upper limit of the size range, 9 cm, may represent the Thalassinoides shafts in the Miocene of southeast Spain. Decreasing the cell size in the 1 m³ 3D volume results in an increase in the number of cells, as the smaller the cell size in the 1 m³ 3D volume, the smaller the size of the Thalassinoides. This yields 1 m³ 3D volumes with 1003, 2003, 3003, 4003, 5003, 6003 cells. In order to examine the results, six images from each of the six sides of the MPS models were extracted, for a total of 108 2D images with 1 m² area. Further, each image represents one face. JMicroVision™ open-source software was used for image analysis to measure the width of the Thalassinoides shafts in each of the 108 images and use it as a proxy for Thalassinoides BS. Descriptive statistics of these measurements were calculated. Statistical parameters such as the mean (μ), median, minima, maxima, and standard deviation (σ) were used to represent the width of the Thalassinoides shaft that may be measured in each MPS model. The μ and σ were used to indicate the size of the Thalassinoides in each MPS model.

### Example 2: Sampling MPS Model

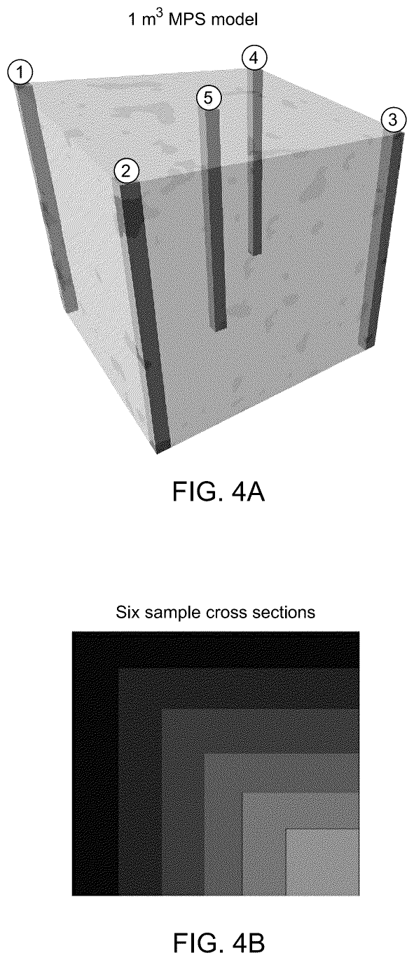

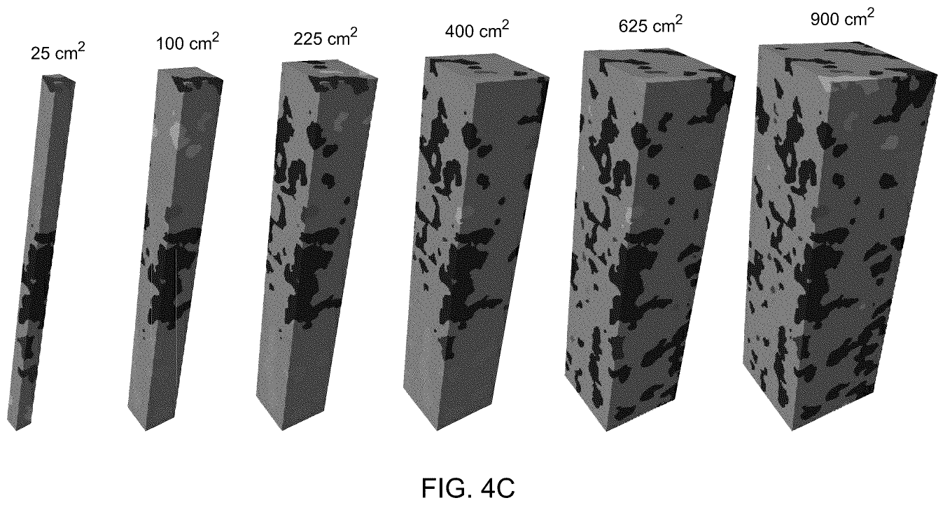

In order to simulate measurements that may be taken from core or image logs, vertical 1 m long columns in four corners and centre of the 18 MPS model cubes were selected as fixed locations for sampling. In particular, five sampling locations were selected, as shown in FIG. 3A and FIG. 4A. A systematic sampling of a randomized modelled Thalassinoides network was performed. At each one of these five sampling locations, six samples were extracted by cropping the model to progressively smaller column cross sections, as shown in FIGS. 4A-4C. An area of a top of the column is the sample cross section (SCS), and the SCS ranges from 25 cm² to 900 cm², by progressively increasing the side length of the top of the column in 5 cm steps, as specified in FIGS. 4B and 4C. Thus, in each 3D model cube (with a single BP and BS), there are 30 columnar samples. This yields a total of 540 digital samples to evaluate the range of BP, BS and SCS. The examination of the results helps for determining the cross-sectional area that is needed for a 1-meter-long sample to fully represent the burrow connectivity (proxy for permeability) from top to bottom of the sample. This is determined for a range of Thalassinoides BP and BS.

### Example 3: Burrow Connectivity

To evaluate sample-scale-dependence of being able to measure connectivity of burrows, separate burrow connectivity analyses was performed on the 540 columnar digital samples as well as on the MPS model cubes. Burrow connectivity was analysed in Petrel™ 2020 using a connected volume function which defines cells that share adjacent faces. The volume of the largest connected burrow volume (LCBV) is used as a proxy for burrow connectivity. In an example, if the LCBV touches the upper and lower faces of the digital cube or columnar sample, then the model has vertical connected burrow volumes and high permeability. This analysis was first performed on the model cubes to verify that the burrows connected across the top and bottom of each model cube. Furthermore, the columnar samples were taken from each cube. For each columnar sample, the burrow connectivity determination was made separately. In order to best simulate measurements on actual samples, the vertical faces of the column were impermeable, and connectivity was only determined from the top to the bottom. Each connected burrow volume was given a unique colour code and code ranked by size, as shown in FIG. 4C. In case the LCBV connected the upper face to the lower face, then the sample measures as permeable and may percolate fluid from top to bottom, as shown in FIG. 4C. Further, in case the LCBV does not touch the upper and lower faces of the sample, then the burrow volumes may not provide permeability pathways from bottom to top of a measured sample, even if the larger sample is known to show such connectivity. Furthermore, two useful parameters for the LCBV are also documented: the length and the position. The length of the LCBV represents its vertical extent, whereas the position of the LCBV represents its location with respect to the top and bottom of the digital sample. For LCBV position, there are four possibilities as follows: touching only the top of the sample; touching only the bottom of the sample; touching both the top and bottom, and not touching the top or bottom.

### Example 4: Probability of Burrow Connectivity

As stated above, in each of the 18 3DMPS models, with BP and BS, 5 digital samples were extracted with the same SCS and were used to determine if LCBV is connected across the upper and lower faces. If the LCBV showed connectivity, as explained in Example 3, the sample was assigned a score of 1. If not, the sample was assigned a score of 0. The probability of a digital sample with a particular BP and BS showing connectivity was calculated as the percentage of the samples with LCBV that showed connectivity from these five samples. A simple example is the five samples which were extracted from the MPS model of 1003 cells (9 cm burrows) and 25% BP. If the connectivity analysis showed that the LCBV in all five samples did not connect through the sample, then the probability of connectivity would be 0%. If all showed connectivity, then the probability of connectivity would be 100%. If three showed connectivity, then the probability of connectivity would be 60%. Probability of connectivity was also expressed by the exceedance probability (EP) of the LCBV length. The lengths of the LCBV were ranked from smallest to largest based on BP, BS and SCS, EP corresponding to each sample was calculated. The results provided three cross plots representing the EP of the ranked length of the LCBV against their actual length. The probability of the samples was calculated from the LCBV length data, specifying whether samples of a particular length may sample the LCBV, given specified SCS, BP, and BS, as provided in FIG. 3C.

### Example 5: Logistic Regression Analysis

In accordance with the present disclosure, binary logistic regression modelling was conducted in order to understand how BP, BS and SCS impact Thalassinoides connectivity of the digital samples, as shown in FIG. 3B. Two categories of connectivity of the LCBV, connected burrow network (given the binary code of 1) and isolated burrow volume (given the binary code 0), were used as a binary dependent variable, whereas the BP, BS and SCS were used as independent variables. The binary logistic regression was performed using XLSTAT software. Results of the binary logistic regression include significance level (P-value) which is used to determine if there is a statistically significant association between the dependent (binary class of Thalassinoides connectivity) and the independent variables (BP, BS and SCS). The results further include beta coefficients (β) which are used to calculate the factor score for the independent variables (BP, BS and SCS) for the probability of Thalassinoides connectivity, and probability equation from logistic regression results, which may be used to calculate the probability of LCBV, in a 1-meter-long sample, connecting across the top and bottom of the sample, for a given BP, BS and SCS.

### Example 6: Validation

The dataset, which includes results from 540 samples, was randomly divided into two subsets. A total of 432 samples (about 80% of the samples) were used as a training set for the regression modelling, whereas 108 samples (about 20% of the samples) were used as a training set for external validation, as shown in Table 1A. The training dataset was used to run the logistic regression analysis, and validation dataset was used to validate the results of the logistic regression.

Validation was conducted by comparing the predicted binary class of the Thalassinoides connectivity versus isolated burrow volume from the logistic regression equation to their actual class. Such comparison helped to evaluate the sensitivity and specificity of the logistic regression results and allowed for assessment if the method can be used as a tool for predicting the Thalassinoides connectivity. For such evaluation, receiver operating characteristics (ROC) method was used, which plots the specificity against 1-specificity of observed versus predicted values. Two approaches for validation were conducted (internal and external). In the internal validation, the training dataset (432 samples) was used for validation, whereas in the external validation, the validation dataset (108 samples) was used. The details of sample sets for training validation, as well other experimental data pertaining to validation is provided in the Tables 1A-1F. It is noteworthy that the provided data is non-limiting in nature.

TABLE 1A
Data sampling (training and validation set - 540 samples)

| BS | BP | SCS | BC | BS | BP | SCS | BC |
|---|---|---|---|---|---|---|---|
| 9 | 75 | 10 | 1 | 9 | 75 | 100 | 1 |
| 2.6 | 50 | 20 | 1 | 2.6 | 50 | 400 | 1 |
| 3.4 | 75 | 25 | 1 | 3.4 | 75 | 625 | 1 |
| 2.6 | 25 | 5 | 0 | 2.6 | 25 | 25 | 0 |
| 2.6 | 25 | 15 | 1 | 2.6 | 25 | 225 | 1 |
| 4 | 25 | 10 | 0 | 4 | 25 | 100 | 0 |
| 2.7 | 25 | 30 | 1 | 2.7 | 25 | 900 | 1 |
| 9 | 75 | 15 | 1 | 9 | 75 | 225 | 1 |
| 9 | 25 | 15 | 0 | 9 | 25 | 225 | 0 |
| 9 | 75 | 20 | 1 | 9 | 75 | 400 | 1 |
| 5.1 | 25 | 30 | 1 | 5.1 | 25 | 900 | 1 |
| 2.6 | 50 | 20 | 1 | 2.6 | 50 | 400 | 1 |
| 2.6 | 75 | 15 | 1 | 2.6 | 75 | 225 | 1 |
| 2.7 | 75 | 30 | 1 | 2.7 | 75 | 900 | 1 |
| 2.7 | 25 | 15 | 1 | 2.7 | 25 | 225 | 1 |
| 2.6 | 25 | 20 | 1 | 2.6 | 25 | 400 | 1 |
| 5.1 | 50 | 30 | 1 | 5.1 | 50 | 900 | 1 |
| 4 | 25 | 20 | 1 | 4 | 25 | 400 | 1 |
| 5.1 | 50 | 5 | 0 | 5.1 | 50 | 25 | 0 |
| 2.7 | 25 | 25 | 1 | 2.7 | 25 | 625 | 1 |
| 2.6 | 75 | 25 | 1 | 2.6 | 75 | 625 | 1 |
| 4 | 50 | 20 | 1 | 4 | 50 | 400 | 1 |
| 2.7 | 25 | 20 | 1 | 2.7 | 25 | 400 | 1 |
| 9 | 25 | 20 | 0 | 9 | 25 | 400 | 0 |
| 2.7 | 50 | 10 | 0 | 2.7 | 50 | 100 | 0 |
| 2.6 | 75 | 5 | 1 | 2.6 | 75 | 25 | 1 |
| 3.4 | 75 | 15 | 1 | 3.4 | 75 | 225 | 1 |
| 9 | 50 | 10 | 0 | 9 | 50 | 100 | 0 |
| 9 | 50 | 10 | 0 | 9 | 50 | 100 | 0 |
| 9 | 50 | 25 | 1 | 9 | 50 | 625 | 1 |
| 2.6 | 25 | 30 | 1 | 2.6 | 25 | 900 | 1 |
| 2.6 | 50 | 10 | 1 | 2.6 | 50 | 100 | 1 |
| 9 | 50 | 20 | 1 | 9 | 50 | 400 | 1 |
| 9 | 50 | 30 | 1 | 9 | 50 | 900 | 1 |
| 5.1 | 25 | 20 | 1 | 5.1 | 25 | 400 | 1 |
| 9 | 50 | 5 | 0 | 9 | 50 | 25 | 0 |
| 5.1 | 25 | 30 | 1 | 5.1 | 25 | 900 | 1 |
| 3.4 | 75 | 25 | 1 | 3.4 | 75 | 625 | 1 |
| 5.1 | 50 | 20 | 1 | 5.1 | 50 | 400 | 1 |
| 5.1 | 50 | 15 | 0 | 5.1 | 50 | 225 | 0 |
| 3.4 | 75 | 20 | 1 | 3.4 | 75 | 400 | 1 |
| 5.1 | 25 | 10 | 0 | 5.1 | 25 | 100 | 0 |
| 3.4 | 50 | 5 | 0 | 3.4 | 50 | 25 | 0 |
| 5.1 | 75 | 10 | 1 | 5.1 | 75 | 100 | 1 |
| 5.1 | 50 | 15 | 0 | 5.1 | 50 | 225 | 0 |
| 4 | 50 | 5 | 0 | 4 | 50 | 25 | 0 |
| 9 | 75 | 15 | 1 | 9 | 75 | 225 | 1 |
| 9 | 25 | 20 | 0 | 9 | 25 | 400 | 0 |
| 4 | 75 | 10 | 1 | 4 | 75 | 100 | 1 |
| 3.4 | 75 | 25 | 1 | 3.4 | 75 | 625 | 1 |
| 4 | 75 | 10 | 1 | 4 | 75 | 100 | 1 |
| 2.6 | 75 | 30 | 1 | 2.6 | 75 | 900 | 1 |
| 5.1 | 75 | 30 | 1 | 5.1 | 75 | 900 | 1 |
| 4 | 50 | 15 | 1 | 4 | 50 | 225 | 1 |
| 2.6 | 50 | 30 | 1 | 2.6 | 50 | 900 | 1 |
| 9 | 50 | 5 | 0 | 9 | 50 | 25 | 0 |
| 3.4 | 50 | 20 | 1 | 3.4 | 50 | 400 | 1 |
| 9 | 75 | 15 | 1 | 9 | 75 | 225 | 1 |
| 3.4 | 50 | 30 | 1 | 3.4 | 50 | 900 | 1 |
| 3.4 | 50 | 5 | 0 | 3.4 | 50 | 25 | 0 |
| 4 | 75 | 15 | 1 | 4 | 75 | 225 | 1 |
| 5.1 | 75 | 20 | 1 | 5.1 | 75 | 400 | 1 |
| 9 | 75 | 30 | 1 | 0 | 75 | 900 | 1 |
| 2.6 | 25 | 10 | 0 | 2.6 | 25 | 100 | 0 |
| 2.7 | 50 | 15 | 1 | 2.7 | 50 | 225 | 1 |
| 3.4 | 50 | 5 | 0 | 3.4 | 50 | 25 | 0 |
| 2.6 | 25 | 10 | 0 | 2.6 | 25 | 100 | 0 |
| 5.1 | 75 | 5 | 0 | 5.1 | 75 | 25 | 0 |
| 2.6 | 25 | 5 | 0 | 2.6 | 25 | 25 | 0 |
| 2.6 | 25 | 0 | 0 | 2.6 | 25 | 100 | 0 |
| 2.6 | 50 | 25 | 1 | 2.6 | 50 | 625 | 1 |
| 2.6 | 25 | 30 | 1 | 2.6 | 25 | 900 | 1 |
| 9 | 75 | 5 | 1 | 9 | 75 | 25 | 1 |
| 2.7 | 75 | 25 | 1 | 2.7 | 75 | 625 | 1 |
| 2.7 | 75 | 25 | 1 | 2.7 | 75 | 625 | 1 |
| 2.7 | 50 | 25 | 1 | 2.7 | 50 | 625 | 1 |
| 2.6 | 25 | 25 | 1 | 2.6 | 25 | 625 | 1 |
| 3.4 | 75 | 10 | 1 | 3.4 | 75 | 100 | 1 |
| 3.4 | 25 | 30 | 1 | 3.4 | 25 | 900 | 1 |
| 2.6 | 75 | 30 | 1 | 2.6 | 75 | 900 | 1 |
| 4 | 75 | 25 | 1 | 4 | 75 | 625 | 1 |
| 2.7 | 75 | 30 | 1 | 2.7 | 75 | 900 | 1 |
| 2.6 | 50 | 20 | 1 | 2.6 | 50 | 400 | 1 |
| 9 | 25 | 20 | 0 | 9 | 25 | 400 | 0 |
| 9 | 25 | 5 | 0 | 9 | 25 | 25 | 0 |
| 5.1 | 75 | 20 | 1 | 5.1 | 75 | 400 | 1 |
| 2.7 | 50 | 5 | 0 | 2.7 | 50 | 25 | 0 |
| 3.4 | 50 | 10 | 1 | 3.4 | 50 | 100 | 1 |
| 2.6 | 50 | 5 | 0 | 2.6 | 50 | 25 | 0 |
| 5.1 | 25 | 5 | 0 | 5.1 | 25 | 25 | 0 |
| 4 | 75 | 15 | 1 | 4 | 75 | 225 | 1 |
| 5.1 | 25 | 5 | 0 | 5.1 | 25 | 25 | 0 |
| 2.7 | 50 | 20 | 1 | 2.7 | 50 | 400 | 1 |
| 2.7 | 75 | 5 | 1 | 2.7 | 75 | 25 | 1 |
| 2.6 | 25 | 25 | 1 | 2.6 | 25 | 625 | 1 |
| 3.4 | 25 | 20 | 1 | 3.4 | 25 | 400 | 1 |
| 2.6 | 50 | 10 | 1 | 2.6 | 50 | 100 | 1 |
| 9 | 75 | 30 | 1 | 9 | 75 | 900 | 1 |
| 2.6 | 25 | 20 | 1 | 2.6 | 25 | 400 | 1 |
| 2.7 | 25 | 5 | 0 | 2.7 | 25 | 25 | 0 |
| 3.4 | 50 | 30 | 1 | 3.4 | 50 | 900 | 1 |
| 9 | 75 | 20 | 1 | 0 | 75 | 400 | 1 |
| 5.1 | 50 | 20 | 1 | 5.1 | 50 | 400 | 1 |
| 9 | 50 | 10 | 0 | 9 | 50 | 100 | 0 |
| 2.6 | 75 | 25 | 1 | 2.6 | 75 | 625 | 1 |
| 5.1 | 75 | 20 | 1 | 5.1 | 75 | 400 | 1 |
| 3.4 | 50 | 15 | 1 | 3.4 | 50 | 225 | 1 |
| 4 | 25 | 15 | 1 | 4 | 25 | 225 | 1 |
| 3.4 | 50 | 15 | 1 | 3.4 | 50 | 225 | 1 |
| 2.6 | 50 | 15 | 1 | 2.6 | 50 | 225 | 1 |
| 4 | 25 | 30 | 1 | 4 | 25 | 900 | 1 |
| 4 | 50 | 15 | 1 | 4 | 50 | 225 | 1 |
| 3.4 | 75 | 10 | 1 | 3.4 | 75 | 100 | 1 |
| 9 | 50 | 15 | 0 | 9 | 50 | 225 | 0 |
| 4 | 25 | 10 | 1 | 4 | 25 | 100 | 1 |
| 3.4 | 25 | 5 | 0 | 3.4 | 25 | 25 | 0 |
| 5.1 | 75 | 30 | 1 | 5.1 | 75 | 900 | 1 |
| 2.6 | 50 | 25 | 1 | 2.6 | 50 | 625 | 1 |
| 5.1 | 50 | 30 | 1 | 5.1 | 50 | 900 | 1 |
| 5.1 | 25 | 25 | 1 | 5.1 | 25 | 625 | 1 |
| 9 | 75 | 25 | 1 | 9 | 75 | 625 | 1 |
| 2.6 | 50 | 5 | 0 | 2.6 | 50 | 25 | 0 |
| 3.4 | 25 | 15 | 0 | 3.4 | 25 | 225 | 0 |
| 9 | 25 | 5 | 0 | 9 | 25 | 25 | 0 |
| 2.6 | 25 | 15 | 1 | 2.6 | 25 | 225 | 1 |
| 9 | 25 | 20 | 0 | 9 | 25 | 400 | 0 |
| 2.7 | 25 | 10 | 0 | 2.7 | 25 | 100 | 0 |
| 5.1 | 50 | 10 | 0 | 5.1 | 50 | 100 | 0 |
| 2.6 | 50 | 15 | 1 | 2.6 | 50 | 225 | 1 |
| 2.7 | 25 | 15 | 1 | 2.7 | 25 | 225 | 1 |
| 2.7 | 25 | 5 | 0 | 2.7 | 25 | 25 | 0 |
| 9 | 75 | 15 | 1 | 9 | 75 | 225 | 1 |
| 2.6 | 75 | 5 | 1 | 2.6 | 75 | 25 | 1 |
| 5.1 | 25 | 5 | 0 | 5.1 | 25 | 25 | 0 |
| 2.7 | 75 | 10 | 1 | 2.7 | 75 | 100 | 1 |
| 5.1 | 25 | 30 | 1 | 5.1 | 25 | 900 | 1 |
| 9 | 25 | 10 | 0 | 9 | 25 | 100 | 0 |
| 2.6 | 75 | 30 | 1 | 2.6 | 75 | 900 | 1 |
| 2.7 | 75 | 30 | 1 | 2.7 | 75 | 900 | 1 |
| 9 | 25 | 30 | 1 | 9 | 25 | 900 | 1 |
| 3.4 | 25 | 5 | 0 | 3.4 | 25 | 25 | 0 |
| 9 | 50 | 15 | 1 | 9 | 50 | 225 | 1 |
| 2.7 | 50 | 25 | 1 | 2.7 | 50 | 625 | 1 |
| 5.1 | 25 | 5 | 0 | 5.1 | 25 | 25 | 0 |
| 2.6 | 75 | 15 | 1 | 2.6 | 75 | 225 | 1 |
| 2.6 | 75 | 10 | 1 | 2.6 | 75 | 100 | 1 |
| 5.1 | 25 | 25 | 1 | 5.1 | 25 | 625 | 1 |
| 3.4 | 75 | 20 | 1 | 3.4 | 75 | 400 | 1 |
| 4 | 25 | 5 | 0 | 4 | 25 | 25 | 0 |
| 2.6 | 75 | 25 | 1 | 2.6 | 75 | 625 | 1 |
| 2.7 | 25 | 25 | 1 | 2.7 | 25 | 625 | 1 |
| 2.6 | 75 | 30 | 1 | 2.6 | 75 | 900 | 1 |
| 9 | 50 | 25 | 0 | 9 | 50 | 625 | 0 |
| 2.7 | 25 | 25 | 1 | 2.7 | 25 | 625 | 1 |
| 2.7 | 75 | 20 | 1 | 2.7 | 75 | 400 | 1 |
| 2.7 | 50 | 25 | 1 | 2.7 | 50 | 625 | 1 |
| 5.1 | 75 | 25 | 1 | 5.1 | 75 | 625 | 1 |
| 3.4 | 50 | 25 | 1 | 3.4 | 50 | 625 | 1 |
| 2.6 | 50 | 30 | 1 | 2.6 | 50 | 900 | 1 |
| 9 | 75 | 5 | 0 | 9 | 75 | 25 | 0 |
| 5.1 | 75 | 25 | 1 | 5.1 | 75 | 625 | 1 |
| 9 | 25 | 15 | 0 | 9 | 25 | 225 | 0 |
| 2.7 | 75 | 15 | 1 | 2.7 | 75 | 225 | 1 |
| 4 | 25 | 30 | 1 | 4 | 25 | 900 | 1 |
| 4 | 75 | 5 | 1 | 4 | 75 | 25 | 1 |
| 9 | 25 | 25 | 0 | 9 | 25 | 625 | 0 |
| 5.1 | 50 | 10 | 0 | 5.1 | 50 | 100 | 0 |
| 2.7 | 75 | 5 | 1 | 2.7 | 75 | 25 | 1 |
| 9 | 25 | 5 | 0 | 9 | 25 | 25 | 0 |
| 3.4 | 25 | 25 | 1 | 3.4 | 25 | 625 | 1 |
| 9 | 25 | 25 | 0 | 9 | 25 | 625 | 0 |
| 3.4 | 75 | 30 | 1 | 3.4 | 75 | 900 | 1 |
| 5.1 | 25 | 20 | 0 | 5.1 | 25 | 400 | 0 |
| 3.4 | 50 | 20 | 1 | 3.4 | 50 | 400 | 1 |
| 9 | 75 | 30 | 1 | 9 | 75 | 900 | 1 |
| 2.7 | 75 | 25 | 1 | 2.7 | 75 | 625 | 1 |
| 4 | 75 | 25 | 1 | 4 | 75 | 625 | 1 |
| 2.6 | 75 | 5 | 1 | 2.6 | 75 | 25 | 1 |
| 9 | 50 | 30 | 1 | 9 | 50 | 900 | 1 |
| 9 | 25 | 15 | 0 | 9 | 25 | 225 | 0 |
| 5.1 | 75 | 5 | 1 | 5.1 | 75 | 25 | 1 |
| 5.1 | 75 | 20 | 1 | 5.1 | 75 | 400 | 1 |
| 4 | 50 | 20 | 1 | 4 | 50 | 400 | 1 |
| 2.7 | 75 | 10 | 1 | 2.7 | 75 | 100 | 1 |
| 5.1 | 50 | 30 | 1 | 5.1 | 50 | 900 | 1 |
| 3.4 | 50 | 25 | 1 | 3.4 | 50 | 625 | 1 |
| 2.7 | 50 | 20 | 1 | 2.7 | 50 | 400 | 1 |
| 5.1 | 25 | 30 | 1 | 5.1 | 25 | 900 | 1 |
| 2.7 | 50 | 15 | 1 | 2.7 | 50 | 225 | 1 |
| 4 | 75 | 10 | 1 | 4 | 75 | 100 | 1 |
| 2.6 | 25 | 10 | 1 | 2.6 | 25 | 100 | 1 |
| 9 | 25 | 30 | 0 | 9 | 25 | 900 | 0 |
| 5.1 | 50 | 10 | 0 | 5.1 | 50 | 100 | 0 |
| 9 | 50 | 5 | 0 | 9 | 50 | 25 | 0 |
| 2.7 | 75 | 30 | 1 | 2.7 | 75 | 900 | 1 |
| 5.1 | 25 | 5 | 0 | 5.1 | 25 | 25 | 0 |
| 4 | 25 | 25 | 1 | 4 | 25 | 625 | 1 |
| 5.1 | 75 | 10 | 1 | 5.1 | 75 | 100 | 1 |
| 3.4 | 75 | 15 | 1 | 3.4 | 75 | 225 | 1 |
| 2.7 | 50 | 30 | 1 | 2.7 | 50 | 900 | 1 |
| 4 | 25 | 10 | 0 | 4 | 25 | 100 | 0 |
| 2.7 | 75 | 15 | 1 | 2.7 | 75 | 225 | 1 |
| 2.6 | 50 | 5 | 0 | 2.6 | 50 | 25 | 0 |
| 2.6 | 75 | 20 | 1 | 2.6 | 75 | 400 | 1 |
| 2.7 | 25 | 20 | 1 | 2.7 | 25 | 400 | 1 |
| 9 | 50 | 25 | 1 | 9 | 50 | 625 | 1 |
| 4 | 75 | 25 | 1 | 4 | 75 | 625 | 1 |
| 9 | 50 | 5 | 0 | 9 | 50 | 25 | 0 |
| 2.7 | 50 | 20 | 1 | 2.7 | 50 | 400 | 1 |
| 2.7 | 25 | 5 | 0 | 2.7 | 25 | 25 | 0 |
| 2.7 | 50 | 5 | 0 | 2.7 | 50 | 25 | 0 |
| 3.4 | 75 | 20 | 1 | 3.4 | 75 | 400 | 1 |
| 2.7 | 75 | 0 | 1 | 2.7 | 75 | 100 | 1 |
| 9 | 50 | 25 | 1 | 9 | 50 | 625 | 1 |
| 9 | 75 | 10 | 0 | 9 | 75 | 100 | 0 |
| 5.1 | 50 | 20 | 1 | 5.1 | 50 | 400 | 1 |
| 5.1 | 50 | 15 | 0 | 5.1 | 50 | 225 | 0 |
| 2.7 | 25 | 20 | 1 | 2.7 | 25 | 400 | 1 |
| 4 | 25 | 15 | 0 | 4 | 25 | 225 | 0 |
| 2.7 | 50 | 10 | 1 | 2.7 | 50 | 100 | 1 |
| 5.1 | 25 | 15 | 0 | 5.1 | 25 | 225 | 0 |
| 9 | 75 | 5 | 0 | 9 | 75 | 25 | 0 |
| 2.7 | 25 | 20 | 1 | 2.7 | 25 | 400 | 1 |
| 3.4 | 75 | 20 | 1 | 3.4 | 75 | 400 | 1 |
| 5.1 | 75 | 30 | 1 | 5.1 | 75 | 900 | 1 |
| 2.6 | 50 | 15 | 1 | 2.6 | 50 | 225 | 1 |
| 4 | 25 | 10 | 0 | 4 | 25 | 100 | 0 |
| 4 | 50 | 5 | 0 | 4 | 50 | 25 | 0 |
| 3.4 | 50 | 15 | 1 | 3.4 | 50 | 225 | 1 |
| 2.6 | 25 | 25 | 1 | 2.6 | 25 | 625 | 1 |
| 2.6 | 50 | 5 | 0 | 2.6 | 50 | 25 | 0 |
| 2.7 | 50 | 5 | 0 | 2.7 | 50 | 25 | 0 |
| 9 | 75 | 5 | 0 | 9 | 75 | 25 | 0 |
| 2.7 | 25 | 15 | 1 | 2.7 | 25 | 225 | 1 |
| 4 | 25 | 30 | 1 | 4 | 25 | 900 | 1 |
| 3.4 | 25 | 10 | 0 | 3.4 | 25 | 100 | 0 |
| 5.1 | 25 | 10 | 0 | 5.1 | 25 | 100 | 0 |
| 2.7 | 75 | 20 | 1 | 2.7 | 75 | 400 | 1 |
| 2.6 | 25 | 5 | 0 | 2.6 | 25 | 25 | 0 |
| 2.7 | 50 | 5 | 0 | 2.7 | 50 | 25 | 0 |
| 4 | 25 | 10 | 1 | 4 | 25 | 100 | 1 |
| 3.4 | 25 | 25 | 1 | 3.4 | 25 | 625 | 1 |
| 5.1 | 75 | 15 | 1 | 5.1 | 75 | 225 | 1 |
| 2.6 | 25 | 15 | 1 | 2.6 | 25 | 225 | 1 |
| 2.6 | 50 | 25 | 1 | 2.6 | 50 | 625 | 1 |
| 3.4 | 75 | 30 | 1 | 3.4 | 75 | 900 | 1 |
| 5.1 | 75 | 30 | 1 | 5.1 | 75 | 900 | 1 |
| 2.6 | 75 | 10 | 1 | 2.6 | 75 | 100 | 1 |
| 4 | 75 | 5 | 0 | 4 | 75 | 25 | 0 |
| 2.6 | 25 | 20 | 1 | 2.6 | 25 | 400 | 1 |
| 3.4 | 50 | 5 | 0 | 3.4 | 50 | 25 | 0 |
| 9 | 25 | 25 | 0 | 9 | 25 | 625 | 0 |
| 5.1 | 25 | 20 | 0 | 5.1 | 25 | 400 | 0 |
| 3.4 | 50 | 20 | 1 | 3.4 | 50 | 400 | 1 |
| 2.7 | 25 | 15 | 0 | 2.7 | 25 | 225 | 0 |
| 5.1 | 25 | 30 | 0 | 5.1 | 25 | 900 | 0 |
| 5.1 | 50 | 25 | 1 | 5.1 | 50 | 625 | 1 |
| 3.4 | 25 | 5 | 0 | 3.4 | 25 | 25 | 0 |
| 4 | 50 | 15 | 1 | 4 | 50 | 225 | 1 |
| 5.1 | 75 | 25 | 1 | 5.1 | 75 | 625 | 1 |
| 9 | 25 | 15 | 0 | 9 | 25 | 225 | 0 |
| 3.4 | 50 | 25 | 1 | 3.4 | 50 | 625 | 1 |
| 5.1 | 25 | 20 | 0 | 5.1 | 25 | 400 | 0 |
| 3.4 | 75 | 25 | 1 | 3.4 | 75 | 625 | 1 |
| 5.1 | 25 | 15 | 0 | 5.1 | 25 | 225 | 0 |
| 5.1 | 25 | 15 | 0 | 5.1 | 25 | 225 | 0 |
| 9 | 50 | 15 | 0 | 9 | 50 | 225 | 0 |
| 2.6 | 50 | 10 | 1 | 2.6 | 50 | 100 | 1 |
| 2.7 | 75 | 5 | 0 | 2.7 | 75 | 25 | 0 |
| 2.7 | 50 | 10 | 0 | 2.7 | 50 | 100 | 0 |
| 9 | 50 | 10 | 0 | 9 | 50 | 100 | 0 |
| 5.1 | 50 | 25 | 1 | 5.1 | 50 | 625 | 1 |
| 2.7 | 75 | 25 | 1 | 2.7 | 75 | 625 | 1 |
| 2.6 | 25 | 20 | 1 | 2.6 | 25 | 400 | 1 |
| 5.1 | 50 | 5 | 0 | 5.1 | 50 | 25 | 0 |
| 3.4 | 50 | 10 | 1 | 3.4 | 50 | 100 | 1 |
| 3.4 | 75 | 25 | 1 | 3.4 | 75 | 625 | 1 |
| 5.1 | 75 | 5 | 0 | 5.1 | 75 | 25 | 0 |
| 2.7 | 25 | 30 | 1 | 2.7 | 25 | 900 | 1 |
| 9 | 50 | 30 | 1 | 9 | 50 | 900 | 1 |
| 2.7 | 25 | 5 | 0 | 2.7 | 25 | 25 | 0 |
| 9 | 50 | 10 | 0 | 9 | 50 | 100 | 0 |
| 5.1 | 50 | 5 | 0 | 5.1 | 50 | 25 | 0 |
| 3.4 | 25 | 20 | 0 | 3.4 | 25 | 400 | 0 |
| 5.1 | 50 | 25 | 1 | 5.1 | 50 | 625 | 1 |
| 2.6 | 50 | 25 | 1 | 2.6 | 50 | 625 | 1 |
| 3.4 | 25 | 5 | 0 | 3.4 | 25 | 25 | 0 |
| 5.1 | 75 | 25 | 1 | 5.1 | 75 | 625 | 1 |
| 2.6 | 25 | 25 | 1 | 2.6 | 25 | 625 | 1 |
| 9 | 75 | 10 | 1 | 9 | 75 | 100 | 1 |
| 2.6 | 50 | 25 | 1 | 2.6 | 50 | 625 | 1 |
| 3.4 | 25 | 20 | 1 | 3.4 | 25 | 400 | 1 |
| 5.1 | 75 | 15 | 1 | 5.1 | 75 | 225 | 1 |
| 3.4 | 25 | 10 | 0 | 3.4 | 25 | 100 | 0 |
| 4 | 50 | 30 | 1 | 4 | 50 | 900 | 1 |
| 4 | 75 | 20 | 1 | 4 | 75 | 400 | 1 |
| 3.4 | 75 | 15 | 1 | 3.4 | 75 | 225 | 1 |
| 2.7 | 25 | 25 | 1 | 2.7 | 25 | 625 | 1 |
| 4 | 50 | 25 | 1 | 4 | 50 | 625 | 1 |
| 5.1 | 75 | 30 | 1 | 5.1 | 75 | 900 | 1 |
| 4 | 50 | 10 | 0 | 4 | 50 | 100 | 0 |
| 2.7 | 25 | 10 | 0 | 2.7 | 25 | 100 | 0 |
| 5.1 | 50 | 10 | 0 | 5.1 | 50 | 100 | 0 |
| 4 | 25 | 20 | 0 | 4 | 25 | 400 | 0 |
| 5.1 | 75 | 10 | 0 | 5.1 | 75 | 100 | 0 |
| 2.6 | 75 | 20 | 1 | 2.6 | 75 | 400 | 1 |
| 2.6 | 75 | 15 | 1 | 2.6 | 75 | 225 | 1 |
| 5.1 | 50 | 30 | 1 | 5.1 | 50 | 900 | 1 |
| 2.7 | 75 | 30 | 1 | 2.7 | 75 | 900 | 1 |
| 2.7 | 75 | 20 | 1 | 2.7 | 75 | 400 | 1 |
| 4 | 50 | 10 | 0 | 4 | 50 | 100 | 0 |
| 9 | 50 | 15 | 0 | 9 | 50 | 225 | 0 |
| 2.7 | 75 | 10 | 1 | 2.7 | 75 | 100 | 1 |
| 2.7 | 50 | 30 | 1 | 2.7 | 50 | 900 | 1 |
| 4 | 25 | 10 | 0 | 4 | 25 | 100 | 0 |
| 3.4 | 75 | 5 | 0 | 3.4 | 75 | 25 | 0 |
| 3.4 | 25 | 25 | 1 | 3.4 | 25 | 625 | 1 |
| 2.7 | 50 | 30 | 1 | 2.7 | 50 | 900 | 1 |
| 5.1 | 25 | 20 | 0 | 5.1 | 25 | 400 | 0 |
| 2.6 | 25 | 5 | 0 | 2.6 | 25 | 25 | 0 |
| 4 | 25 | 20 | 1 | 4 | 25 | 400 | 1 |
| 3.4 | 75 | 10 | 1 | 3.4 | 75 | 100 | 1 |
| 3.4 | 25 | 30 | 1 | 3.4 | 25 | 900 | 1 |
| 2.7 | 50 | 30 | 1 | 2.7 | 50 | 900 | 1 |
| 2.7 | 50 | 25 | 1 | 2.7 | 50 | 625 | 1 |
| 5.1 | 25 | 25 | 0 | 5.1 | 25 | 625 | 0 |
| 2.6 | 50 | 5 | 0 | 2.6 | 50 | 25 | 0 |
| 4 | 75 | 30 | 1 | 4 | 75 | 900 | 1 |
| 2.6 | 50 | 10 | 1 | 2.6 | 50 | 100 | 1 |
| 4 | 50 | 25 | 1 | 4 | 50 | 625 | 1 |
| 2.7 | 75 | 10 | 1 | 2.7 | 75 | 100 | 1 |
| 2.6 | 75 | 15 | 1 | 2.6 | 75 | 225 | 1 |
| 5.1 | 75 | 15 | 1 | 5.1 | 75 | 225 | 1 |
| 5.1 | 25 | 25 | 0 | 5.1 | 25 | 625 | 0 |
| 2.6 | 75 | 15 | 1 | 2.6 | 75 | 225 | 1 |
| 9 | 50 | 20 | 1 | 9 | 50 | 400 | 1 |
| 5.1 | 25 | 10 | 0 | 5.1 | 25 | 100 | 0 |
| 9 | 50 | 15 | 0 | 9 | 50 | 225 | 0 |
| 2.7 | 25 | 20 | 1 | 2.7 | 25 | 400 | 1 |
| 9 | 25 | 25 | 0 | 9 | 25 | 625 | 0 |
| 3.4 | 75 | 5 | 1 | 3.4 | 75 | 25 | 1 |
| 4 | 25 | 15 | 0 | 4 | 25 | 225 | 0 |
| 3.4 | 25 | 25 | 1 | 3.4 | 25 | 625 | 1 |
| 2.7 | 75 | 20 | 1 | 2.7 | 75 | 400 | 1 |
| 2.6 | 25 | 30 | 1 | 2.6 | 25 | 900 | 1 |
| 2.7 | 50 | 15 | 1 | 2.7 | 50 | 225 | 1 |
| 9 | 25 | 25 | 0 | 9 | 25 | 625 | 0 |
| 3.4 | 25 | 15 | 0 | 3.4 | 25 | 225 | 0 |
| 3.4 | 75 | 5 | 1 | 3.4 | 75 | 25 | 1 |
| 9 | 25 | 30 | 0 | 9 | 25 | 900 | 0 |
| 5.1 | 75 | 25 | 1 | 5.1 | 75 | 625 | 1 |
| 5.1 | 50 | 15 | 1 | 5.1 | 50 | 225 | 1 |
| 3.4 | 50 | 15 | 1 | 3.4 | 50 | 225 | 1 |
| 4 | 50 | 10 | 0 | 4 | 50 | 100 | 0 |
| 5.1 | 25 | 15 | 1 | 5.1 | 25 | 225 | 1 |
| 2.7 | 50 | 20 | 1 | 2.7 | 50 | 400 | 1 |
| 5.1 | 50 | 30 | 1 | 5.1 | 50 | 900 | 1 |
| 3.4 | 50 | 15 | 1 | 3.4 | 50 | 225 | 1 |
| 3.4 | 75 | 5 | 0 | 3.4 | 75 | 25 | 0 |
| 2.6 | 25 | 30 | 1 | 2.6 | 25 | 900 | 1 |
| 4 | 75 | 20 | 1 | 4 | 75 | 400 | 1 |
| 4 | 75 | 20 | 1 | 4 | 75 | 400 | 1 |
| 4 | 50 | 30 | 1 | 4 | 50 | 900 | 1 |
| 2.6 | 50 | 20 | 1 | 2.6 | 50 | 400 | 1 |
| 3.4 | 50 | 25 | 1 | 3.4 | 50 | 625 | 1 |
| 2.7 | 25 | 5 | 0 | 2.7 | 25 | 25 | 0 |
| 3.4 | 75 | 20 | 1 | 3.4 | 75 | 400 | 1 |
| 2.6 | 50 | 15 | 1 | 2.6 | 50 | 225 | 1 |
| 9 | 75 | 25 | 1 | 9 | 75 | 625 | 1 |
| 4 | 75 | 30 | 1 | 4 | 75 | 900 | 1 |
| 9 | 25 | 15 | 0 | 9 | 25 | 225 | 0 |
| 3.4 | 50 | 25 | 1 | 3.4 | 50 | 625 | 1 |
| 2.7 | 50 | 5 | 0 | 2.7 | 50 | 25 | 0 |
| 3.4 | 25 | 10 | 0 | 3.4 | 25 | 100 | 0 |
| 4 | 50 | 15 | 1 | 4 | 50 | 225 | 1 |
| 9 | 50 | 5 | 0 | 9 | 50 | 25 | 0 |
| 2.7 | 75 | 5 | 1 | 2.7 | 75 | 25 | 1 |
| 5.1 | 75 | 10 | 1 | 5.1 | 75 | 100 | 1 |
| 3.4 | 25 | 15 | 1 | 3.4 | 25 | 225 | 1 |
| 3.4 | 25 | 30 | 1 | 3.4 | 25 | 900 | 1 |
| 4 | 50 | 20 | 1 | 4 | 50 | 400 | 1 |
| 2.6 | 75 | 5 | 1 | 2.6 | 75 | 25 | 1 |
| 4 | 50 | 5 | 0 | 4 | 50 | 25 | 0 |
| 9 | 25 | 30 | 1 | 9 | 25 | 900 | 1 |
| 4 | 75 | 10 | 1 | 4 | 75 | 100 | 1 |
| 2.6 | 75 | 20 | 1 | 2.6 | 75 | 400 | 1 |
| 3.4 | 25 | 30 | 1 | 3.4 | 25 | 900 | 1 |
| 4 | 75 | 25 | 1 | 4 | 75 | 625 | 1 |
| 3.4 | 75 | 30 | 1 | 3.4 | 75 | 900 | 1 |
| 3.4 | 75 | 15 | 1 | 3.4 | 75 | 225 | 1 |
| 2.6 | 25 | 15 | 0 | 2.6 | 25 | 225 | 0 |
| 2.6 | 75 | 25 | 1 | 2.6 | 75 | 625 | 1 |
| 4 | 75 | 10 | 1 | 4 | 75 | 100 | 1 |
| 5.1 | 50 | 15 | 1 | 5.1 | 50 | 225 | 1 |
| 4 | 25 | 25 | 1 | 4 | 25 | 625 | 1 |
| 4 | 25 | 5 | 0 | 4 | 25 | 25 | 0 |
| 2.6 | 25 | 10 | 1 | 2.6 | 25 | 100 | 1 |
| 4 | 50 | 25 | 1 | 4 | 50 | 625 | 1 |
| 5.1 | 50 | 20 | 1 | 5.1 | 50 | 400 | 1 |
| 3.4 | 25 | 10 | 0 | 3.4 | 25 | 100 | 0 |
| 4 | 25 | 25 | 1 | 4 | 25 | 625 | 1 |
| 5.1 | 75 | 10 | 0 | 5.1 | 75 | 100 | 0 |
| 2.6 | 25 | 15 | 1 | 2.6 | 25 | 225 | 1 |
| 3.4 | 50 | 10 | 1 | 3.4 | 50 | 100 | 1 |
| 3.4 | 75 | 10 | 1 | 3.4 | 75 | 100 | 1 |
| 2.6 | 75 | 20 | 1 | 2.6 | 75 | 400 | 1 |
| 4 | 75 | 25 | 1 | 4 | 75 | 625 | 1 |
| 9 | 75 | 5 | 1 | 9 | 75 | 25 | 1 |
| 3.4 | 75 | 15 | 1 | 3.4 | 75 | 225 | 1 |
| 2.7 | 50 | 20 | 1 | 2.7 | 50 | 400 | 1 |
| 3.4 | 75 | 30 | 1 | 3.4 | 75 | 900 | 1 |
| 4 | 50 | 25 | 1 | 4 | 50 | 625 | 1 |
| 3.4 | 50 | 30 | 1 | 3.4 | 50 | 900 | 1 |
| 4 | 75 | 30 | 1 | 4 | 75 | 900 | 1 |
| 4 | 75 | 30 | 1 | 4 | 75 | 900 | 1 |
| 4 | 75 | 5 | 0 | 4 | 75 | 25 | 0 |
| 9 | 75 | 15 | 1 | 9 | 75 | 225 | 1 |
| 2.6 | 25 | 25 | 1 | 2.6 | 25 | 625 | 1 |
| 9 | 25 | 10 | 0 | 9 | 25 | 100 | 0 |
| 4 | 75 | 5 | 0 | 4 | 75 | 25 | 0 |
| 2.6 | 25 | 30 | 1 | 2.6 | 25 | 900 | 1 |
| 9 | 50 | 20 | 0 | 9 | 50 | 400 | 0 |
| 2.6 | 50 | 15 | 1 | 2.6 | 50 | 225 | 1 |
| 5.1 | 75 | 15 | 1 | 5.1 | 75 | 225 | 1 |
| 9 | 75 | 30 | 1 | 9 | 75 | 900 | 1 |
| 3.4 | 25 | 15 | 1 | 3.4 | 25 | 225 | 1 |
| 3.4 | 25 | 10 | 0 | 3.4 | 25 | 100 | 0 |
| 5.1 | 50 | 5 | 0 | 5.1 | 50 | 25 | 0 |
| 2.7 | 75 | 15 | 1 | 2.7 | 75 | 225 | 1 |
| 2.6 | 75 | 30 | 1 | 2.6 | 75 | 900 | 1 |
| 2.6 | 75 | 10 | 1 | 2.6 | 75 | 100 | 1 |
| 2.7 | 75 | 20 | 1 | 2.7 | 75 | 400 | 1 |
| 5.1 | 25 | 15 | 0 | 5.1 | 25 | 225 | 0 |
| 4 | 50 | 5 | 0 | 4 | 50 | 25 | 0 |
| 4 | 75 | 15 | 1 | 4 | 75 | 225 | 1 |
| 2.6 | 75 | 25 | 1 | 2.6 | 75 | 625 | 1 |
| 4 | 75 | 15 | 1 | 4 | 75 | 225 | 1 |
| 2.6 | 75 | 5 | 1 | 2.6 | 75 | 25 | 1 |
| 3.4 | 25 | 15 | 0 | 3.4 | 25 | 225 | 0 |
| 9 | 25 | 30 | 0 | 9 | 25 | 900 | 0 |
| 9 | 25 | 20 | 0 | 9 | 25 | 400 | 0 |
| 4 | 50 | 20 | 1 | 4 | 50 | 400 | 1 |
| 4 | 75 | 20 | 1 | 4 | 75 | 400 | 1 |
| 3.4 | 25 | 20 | 1 | 3.4 | 25 | 400 | 1 |
| 2.6 | 50 | 10 | 1 | 2.6 | 50 | 100 | 1 |
| 2.7 | 50 | 10 | 0 | 2.7 | 50 | 100 | 0 |
| 4 | 75 | 20 | 1 | 4 | 75 | 400 | 1 |
| 2.6 | 25 | 20 | 1 | 2.6 | 25 | 400 | 1 |
| 2.7 | 50 | 15 | 1 | 2.7 | 50 | 225 | 1 |
| 5.1 | 25 | 10 | 0 | 5.1 | 25 | 100 | 0 |
| 9 | 75 | 20 | 1 | 9 | 75 | 400 | 1 |
| 9 | 25 | 10 | 0 | 9 | 25 | 100 | 0 |
| 5.1 | 75 | 5 | 0 | 5.1 | 75 | 25 | 0 |
| 9 | 50 | 30 | 1 | 9 | 50 | 900 | 1 |
| 4 | 25 | 5 | 0 | 4 | 25 | 25 | 0 |
| 3.4 | 75 | 5 | 1 | 3.4 | 75 | 25 | 1 |
| 2.7 | 25 | 30 | 1 | 2.7 | 25 | 900 | 1 |
| 4 | 75 | 15 | 1 | 4 | 75 | 225 | 1 |
| 2.7 | 50 | 30 | 1 | 2.7 | 50 | 900 | 1 |
| 9 | 75 | 30 | 1 | 9 | 75 | 900 | 1 |
| 4 | 25 | 30 | 1 | 4 | 25 | 900 | 1 |
| 4 | 25 | 5 | 0 | 4 | 25 | 25 | 0 |
| 2.7 | 75 | 15 | 1 | 2.7 | 75 | 225 | 1 |
| 2.7 | 25 | 30 | 1 | 2.7 | 25 | 900 | 1 |
| 9 | 75 | 10 | 1 | 9 | 75 | 100 | 1 |
| 4 | 50 | 5 | 0 | 4 | 50 | 25 | 0 |
| 9 | 75 | 25 | 1 | 9 | 75 | 625 | 1 |
| 2.6 | 25 | 5 | 0 | 2.6 | 25 | 25 | 0 |
| 2.6 | 50 | 20 | 1 | 2.6 | 50 | 400 | 1 |
| 2.6 | 75 | 10 | 1 | 2.6 | 75 | 100 | 1 |
| 4 | 25 | 15 | 0 | 4 | 25 | 225 | 0 |
| 4 | 50 | 20 | 1 | 4 | 50 | 400 | 1 |
| 3.4 | 25 | 25 | 1 | 3.4 | 25 | 625 | 1 |
| 9 | 75 | 10 | 1 | 9 | 75 | 100 | 1 |
| 3.4 | 50 | 5 | 0 | 3.4 | 50 | 25 | 0 |
| 5.1 | 75 | 15 | 1 | 5.1 | 75 | 225 | 1 |
| 5.1 | 50 | 20 | 1 | 5.1 | 50 | 400 | 1 |
| 2.6 | 75 | 20 | 1 | 2.6 | 75 | 400 | 1 |
| 9 | 75 | 20 | 1 | 9 | 75 | 400 | 1 |
| 3.4 | 50 | 10 | 1 | 3.4 | 50 | 100 | 1 |
| 5.1 | 25 | 25 | 1 | 5.1 | 25 | 625 | 1 |
| 4 | 75 | 30 | 1 | 4 | 75 | 900 | 1 |
| 4 | 25 | 30 | 1 | 4 | 25 | 900 | 1 |
| 9 | 25 | 10 | 0 | 9 | 25 | 100 | 0 |
| 4 | 50 | 25 | 1 | 4 | 50 | 625 | 1 |
| 2.7 | 75 | 15 | 1 | 2.7 | 75 | 225 | 1 |
| 3.4 | 25 | 20 | 1 | 3.4 | 25 | 400 | 1 |
| 3.4 | 50 | 20 | 1 | 3.4 | 50 | 400 | 1 |
| 4 | 25 | 25 | 0 | 4 | 25 | 625 | 0 |
| 2.7 | 25 | 30 | 1 | 2.7 | 25 | 900 | 1 |
| 3.4 | 50 | 20 | 1 | 3.4 | 50 | 400 | 1 |
| 3.4 | 75 | 10 | 1 | 3.4 | 75 | 100 | 1 |
| 2.7 | 50 | 25 | 1 | 2.7 | 50 | 625 | 1 |
| 9 | 25 | 5 | 0 | 9 | 25 | 25 | 0 |
| 4 | 50 | 30 | 1 | 4 | 50 | 900 | 1 |
| 9 | 25 | 5 | 0 | 9 | 25 | 25 | 0 |
| 2.7 | 25 | 15 | 1 | 2.7 | 25 | 225 | 1 |
| 5.1 | 50 | 5 | 0 | 5.1 | 50 | 25 | 0 |
| 4 | 25 | 15 | 0 | 4 | 25 | 225 | 0 |
| 9 | 50 | 20 | 0 | 9 | 50 | 400 | 0 |
| 5.1 | 50 | 25 | 1 | 5.1 | 50 | 625 | 1 |
| 4 | 25 | 25 | 1 | 4 | 25 | 625 | 1 |
| 5.1 | 75 | 20 | 1 | 5.1 | 75 | 400 | 1 |
| 4 | 25 | 20 | 1 | 4 | 25 | 400 | 1 |
| 9 | 75 | 25 | 1 | 9 | 75 | 625 | 1 |
| 3.4 | 50 | 30 | 1 | 3.4 | 50 | 900 | 1 |
| 2.7 | 25 | 25 | 1 | 2.7 | 25 | 625 | 1 |
| 9 | 50 | 20 | 0 | 9 | 50 | 400 | 0 |
| 9 | 25 | 10 | 0 | 9 | 25 | 100 | 0 |
| 9 | 50 | 30 | 1 | 9 | 50 | 900 | 1 |
| 9 | 50 | 25 | 1 | 9 | 50 | 625 | 1 |
| 3.4 | 50 | 30 | 1 | 3.4 | 50 | 900 | 1 |
| 2.6 | 50 | 30 | 1 | 2.6 | 50 | 900 | 1 |
| 4 | 25 | 20 | 1 | 4 | 25 | 400 | 1 |
| 2.7 | 25 | 10 | 0 | 2.7 | 25 | 100 | 0 |
| 3.4 | 25 | 5 | 0 | 3.4 | 25 | 25 | 0 |
| 5.1 | 75 | 5 | 0 | 5.1 | 75 | 25 | 0 |
| 4 | 25 | 5 | 0 | 4 | 25 | 25 | 0 |
| 4 | 75 | 5 | 0 | 4 | 75 | 25 | 0 |
| 2.6 | 50 | 30 | 1 | 2.6 | 50 | 900 | 1 |
| 2.7 | 25 | 10 | 0 | 2.7 | 25 | 100 | 0 |
| 4 | 50 | 10 | 1 | 4 | 50 | 100 | 1 |
| 3.4 | 50 | 10 | 1 | 3.4 | 50 | 100 | 1 |
| 2.6 | 50 | 30 | 1 | 2.6 | 50 | 900 | 1 |
| 3.4 | 25 | 30 | 1 | 3.4 | 25 | 900 | 1 |
| 2.7 | 75 | 5 | 0 | 2.7 | 75 | 25 | 0 |
| 2.7 | 25 | 10 | 0 | 2.7 | 25 | 100 | 0 |
| 4 | 50 | 30 | 1 | 4 | 50 | 900 | 1 |
| 5.1 | 50 | 25 | 1 | 5.1 | 50 | 625 | 1 |
| 4 | 50 | 15 | 1 | 4 | 50 | 225 | 1 |
| 4 | 50 | 10 | 0 | 4 | 50 | 100 | 0 |
| 5.1 | 50 | 10 | 0 | 5.1 | 50 | 100 | 0 |
| 2.7 | 50 | 15 | 1 | 2.7 | 50 | 225 | 1 |
| 4 | 50 | 30 | 1 | 4 | 50 | 900 | 1 |
| 2.7 | 75 | 25 | 1 | 2.7 | 75 | 625 | 1 |
| 9 | 75 | 25 | 1 | 9 | 75 | 625 | 1 |
| 9 | 75 | 20 | 1 | 9 | 75 | 400 | 1 |
| 2.6 | 75 | 10 | 1 | 2.6 | 75 | 100 | 1 |
| 3.4 | 75 | 30 | 1 | 3.4 | 75 | 900 | 1 |
| 2.7 | 50 | 10 | 1 | 2.7 | 50 | 100 | 1 |

TABLE 1B
Summary statistics (quantitative data)

| | | Obs. | Obs. | | | | |
|---|---|---|---|---|---|---|---|
| | | with | without | | | | |
| | | missing | missing | | | | Std. |
| Variable | Observations | data | data | Minimum | Maximum | Mean | deviation |
| BS | 432 | 0 | 432 | 2.600 | 9.000 | 4.422 | 2.177 |
| BP | 432 | 0 | 432 | 25.00 | 75.000 | 50.289 | 20.540 |
| SCS | 432 | 0 | 432 | 25.00 | 900.000 | 376.157 | 303.477 |

TABLE 1C
Summary statistics (quantitative data/validation)

| | | Obs. | Obs. | | | | |
|---|---|---|---|---|---|---|---|
| | | with | without | | | | |
| | | missing | missing | | | | Std. |
| Variable | Observations | data | data | Minimum | Maximum | Mean | deviation |
| BS | 108 | 0 | 108 | 2.600 | 9.000 | 4.633 | 2.279 |
| BP | 108 | 0 | 108 | 25.000 | 75.000 | 48.843 | 20.042 |
| SCS | 108 | 0 | 108 | 25.000 | 900.000 | 391.204 | 315.049 |

TABLE 1D
Summary statistics (qualitative data)

| Variable | Categories | Counts | Frequencies | % |
|---|---|---|---|---|
| BC | 0 | 139 | 139 | 32.176 |
| | 1 | 293 | 293 | 67.824 |

TABLE 1E
Summary statistics (Qualitative data/validation)

| Variable | Categories | Counts | Frequencies | % |
|---|---|---|---|---|
| BC | 0 | 34 | 34 | 31.481 |
| | 1 | 74 | 74 | 68.519 |

TABLE 1F
Correlation matrix

| | BS | BP | SCS | BC |
|---|---|---|---|---|
| BS | 1 | −0.019 | −0.008 | −0.303 |
| BP | −0.019 | 1 | 0.012 | 0.384 |
| SCS | −0.008 | 0.012 | 1 | 0.496 |
| BC | −0.303 | 0.384 | 0.496 | 1 |

### Example 7: Thalassinoides MPS Models

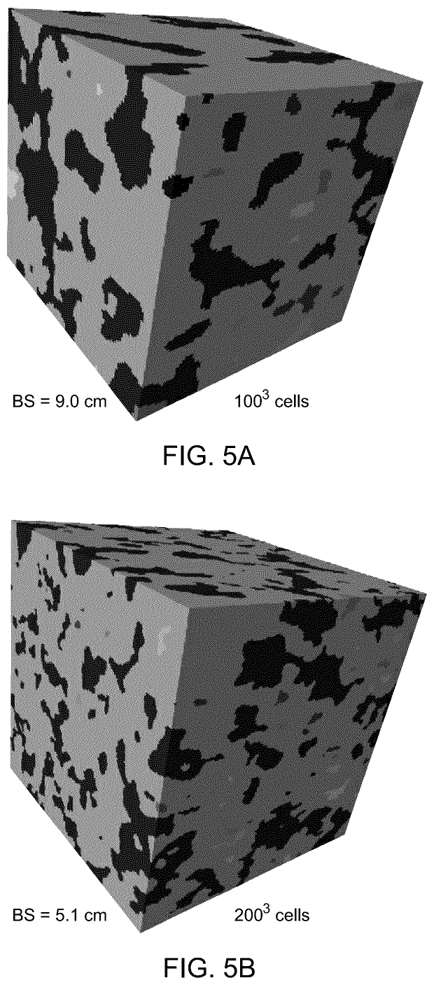

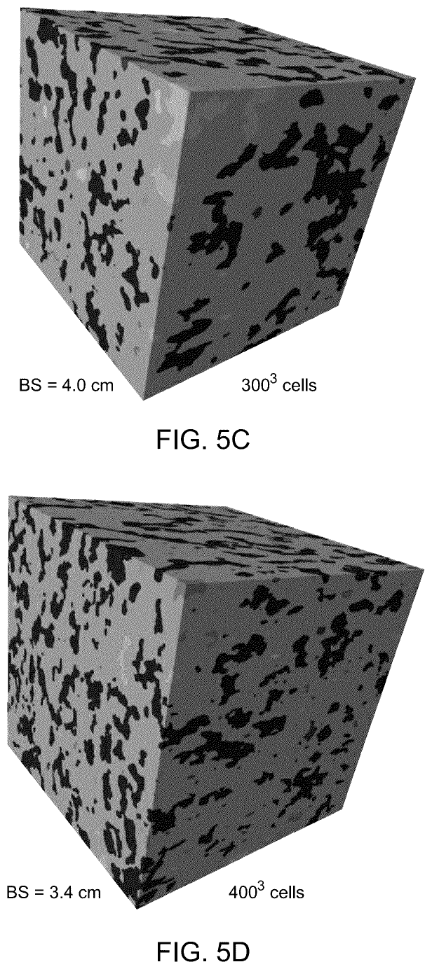

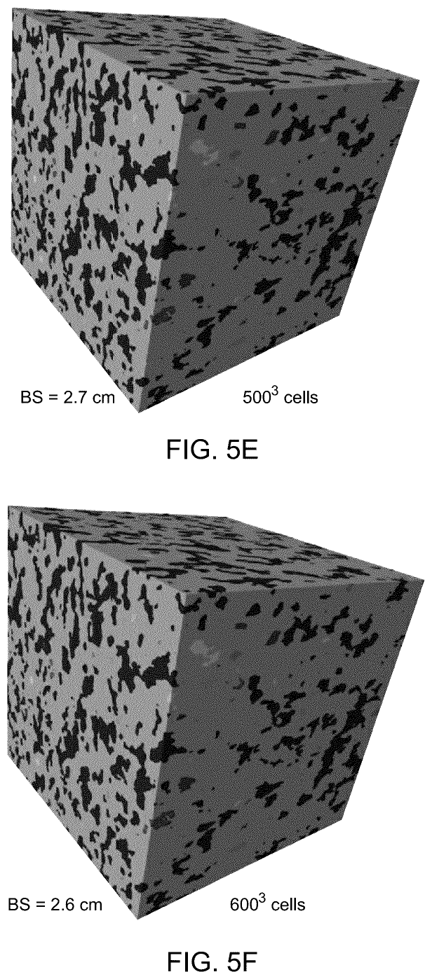

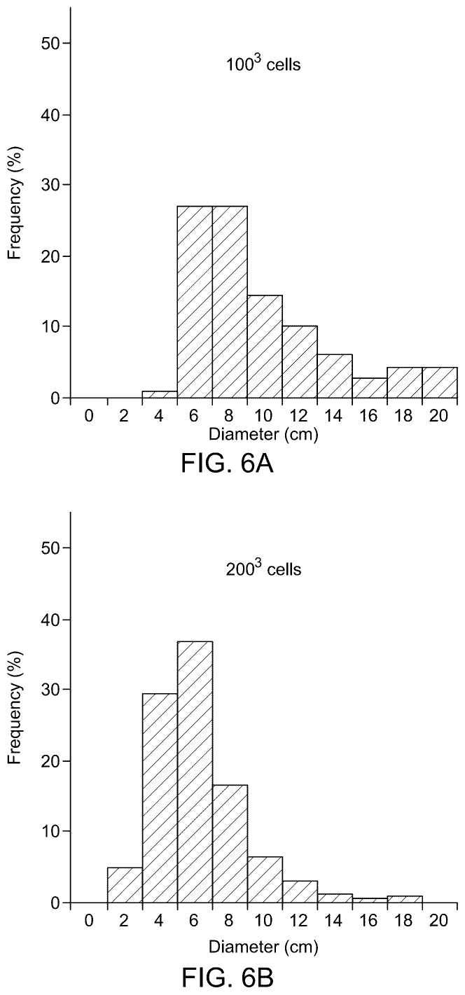

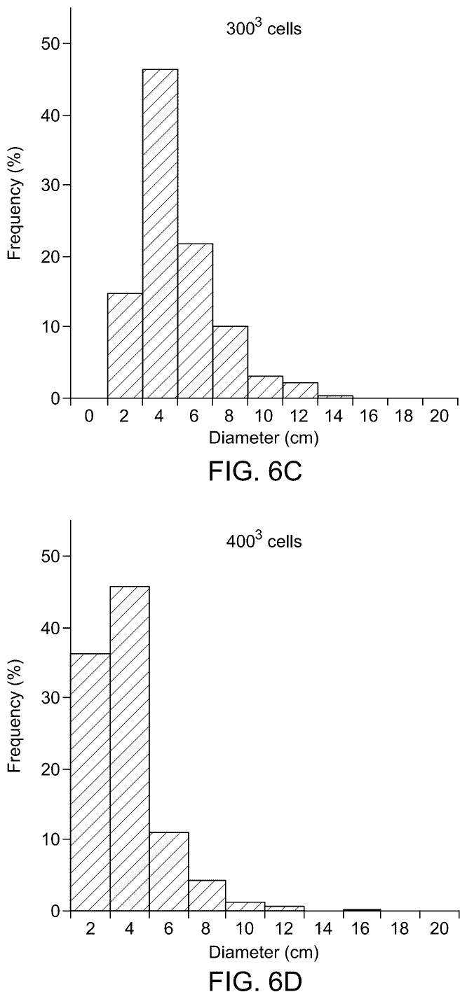

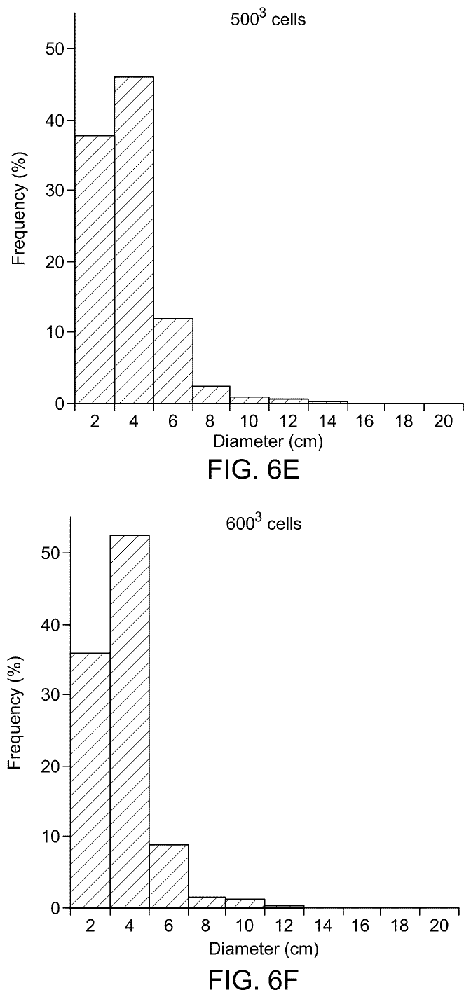

The MPS modelling produces variation in BS by varying cell size of the models (consequently increasing cell number), as shown in FIGS. 5A-5F, FIGS. 6A-6F and Table 2. Results of image analysis using JMicroVision™ help to evaluate BS of the modelled Thalassinoides. For example, the measured diameter of Thalassinoides (BS) on the images of the model with 1003 cells yields a mean of 9.0±4.6 cm; analysis of images from the 6003-cell model yields a mean BS of 2.6±1.3 cm, as listed in Table 2. Histograms of the measured diameter of the modelled Thalassinoides showed progressively smaller ranges, while increasing the number of the cells in the MPS models, as shown in FIGS. 6A-6F. This result is in line with the observation that the Thalassinoides network is better represented in the 1 m³ models, when cell size is large, and BS is small. Table 2 shows the mean of the measured diameter of Thalassinoides in each MPS model, which is used as a proxy for BS in the following sections.

TABLE 2
Mean of the cross section of Thalassinoides
network in each MPS model

| Cell size of | Mean of the Thalassinoides |
|---|---|
| the MPS model | diameter (cm) |
| 100 | 9 |
| 200 | 5.1 |
| 300 | 4 |
| 400 | 3.4 |
| 500 | 2.7 |
| 600 | 2.6 |

### Example 8: Thalassinoides Network and Connectivity

Results of connectivity analyses that were run on the full 1 m³ volumes of the 18 MPS models indicate that the Thalassinoides in these models connects across the top and bottom of the 1 m³ 3D models, as shown in FIGS. 5A-5F. In each model, more than 95% of the total volume of Thalassinoides was connected as part of a LCBV. When the same analysis was run on the 540 columnar samples, taken from these 18 MPS models, however, 173 of the samples (about 32%) showed no top-to-bottom connectivity for the Thalassinoides. This clearly indicates that there is scale dependence in how Thalassinoides connectivity must be sampled.

### Example 9: Controls on LCBV of Thalassinoides

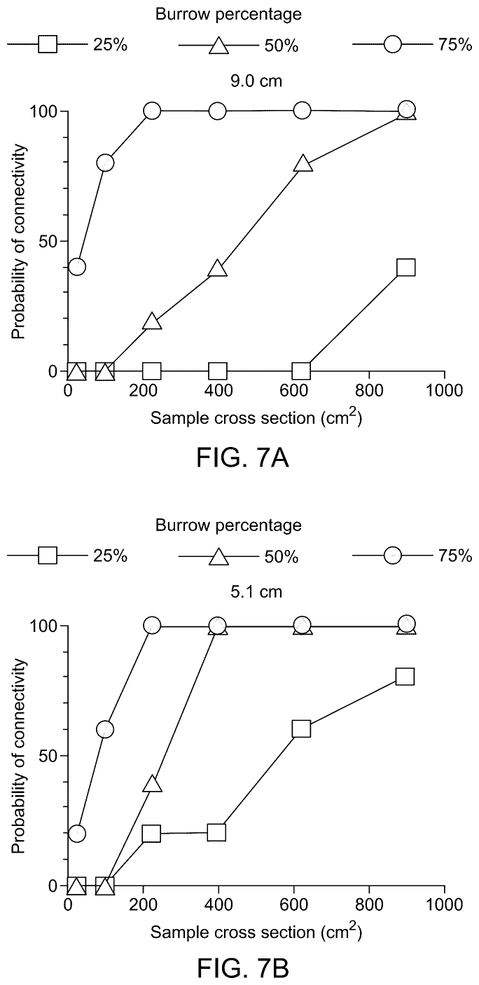

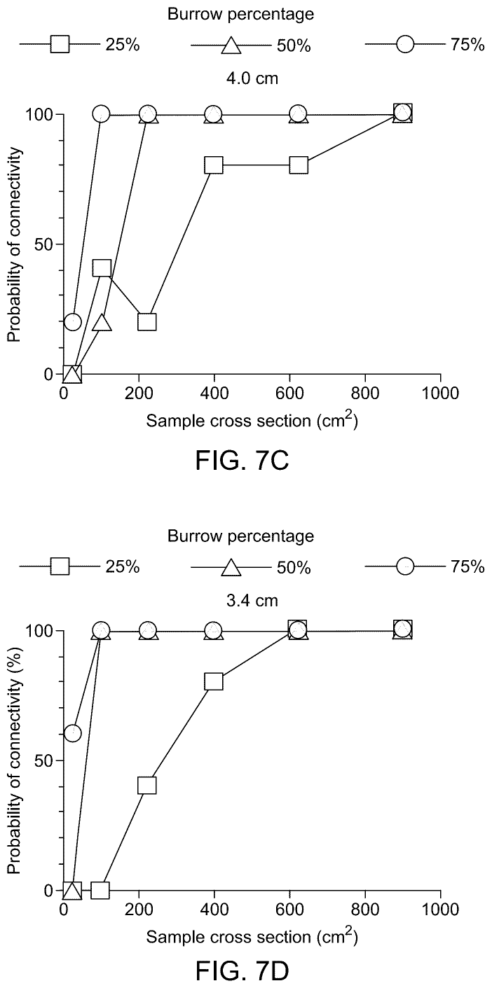

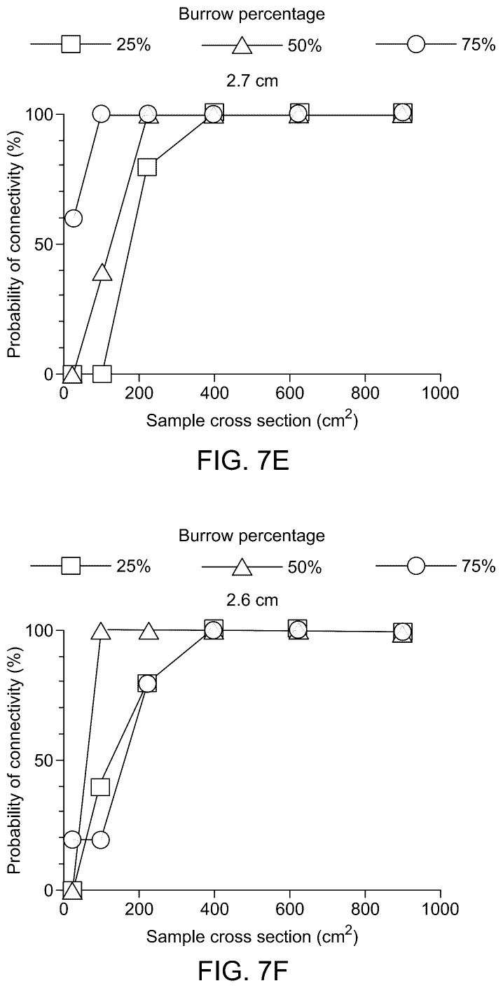

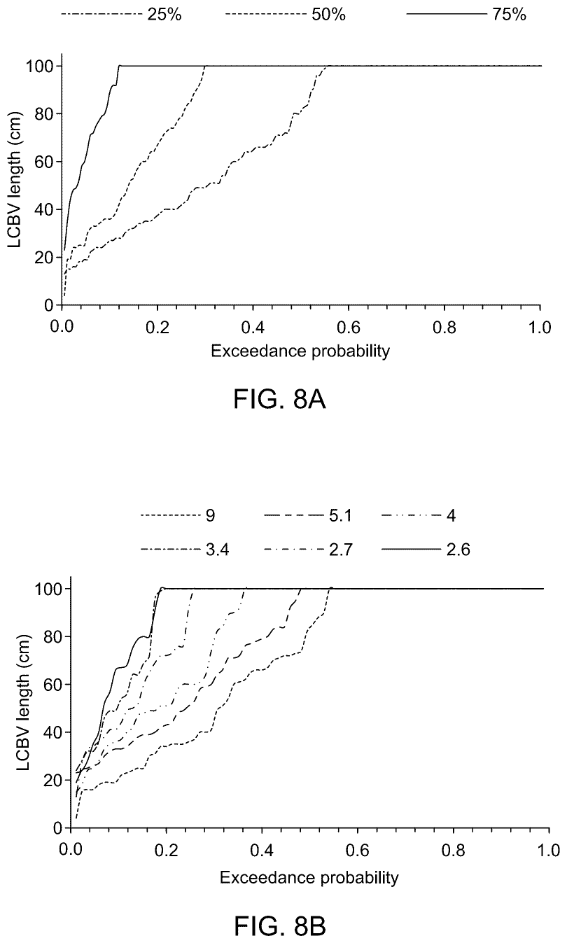

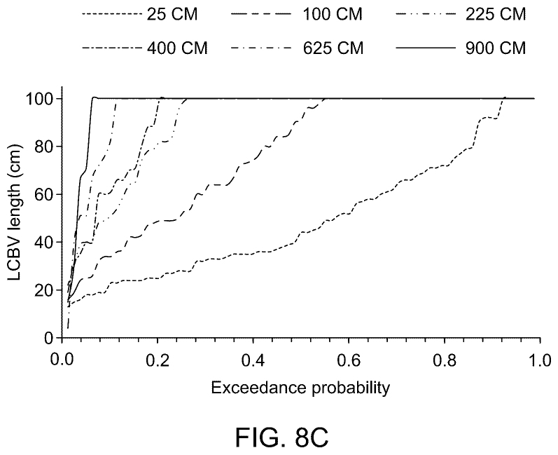

The BS, BP, and SCS controls on probability of Thalassinoides connectivity in the 540 samples may be illustrated graphically, as shown in FIGS. 7A-7F. The results show that for a given BS and SCS, increasing BP results in increased probability of Thalassinoides samples showing connectivity. In contrast, the results show that for a given BP and SCS, increased BS decreases the probability of samples showing the Thalassinoides connectivity, as listed in Table 3A and 3B. Also, for a given BP and BS, increasing the SCS results in an increased probability of samples showing Thalassinoides connectivity, as shown in FIGS. 7A-7F, Table 3A and Table 3B. Similar results may be shown in cross plots of the lengths of the LCBV, as shown in Table 3A and 3B, Tables 4A-4D, and Tables 5A-5D against their EP depicted in FIGS. 8A-8C. In these plots, the curves flatten out at 100 cm where the LCBV of Thalassinoides has reached the maximum length, showing vertical connectivity in the sample. It may be noted in these plots that the point at which the LCBV of Thalassinoides reaches the 100 cm flattening varies depending on the BP, BS and SCS. These graphical analyses of the results visually highlight the importance of BP, BS, and sample size as variables to allow Thalassinoides connectivity to be sampled and properly represented.

### Example 10: Binary Logistic Regression Analysis (Probability of Connectivity)

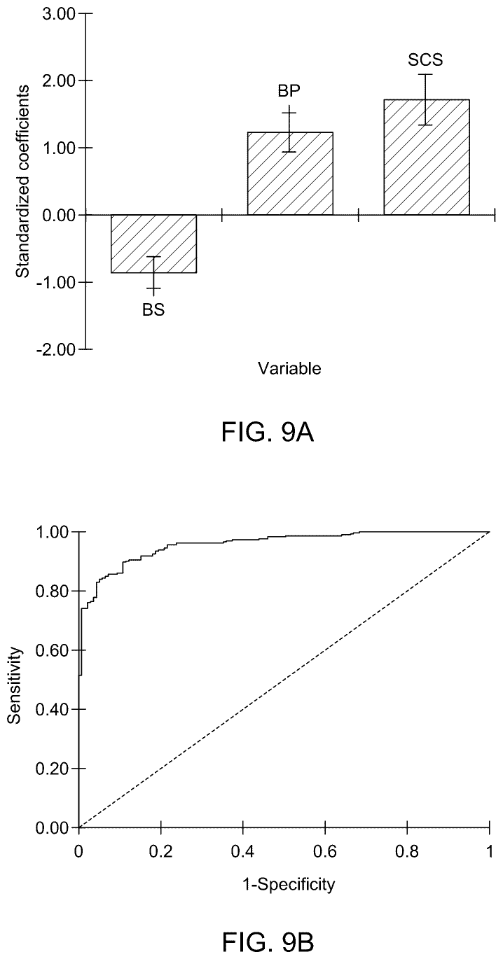

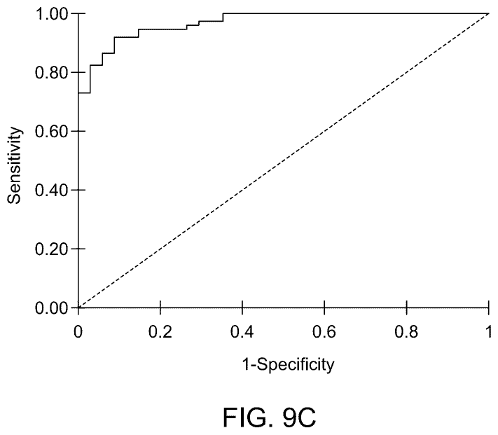

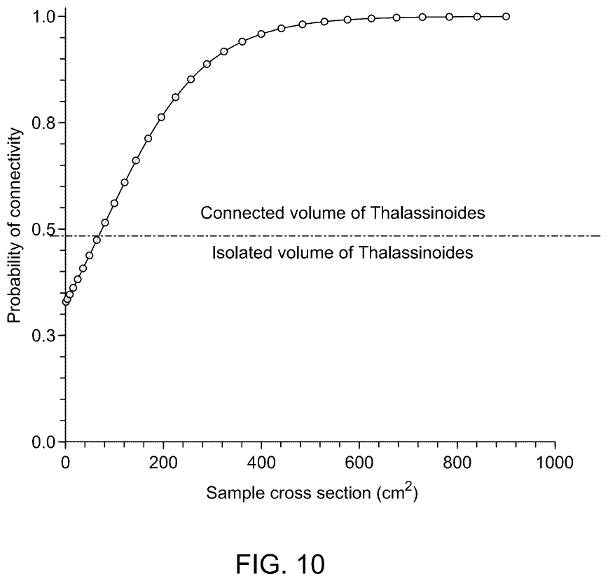

The results of binary logistic regression confirm and quantify the graphic analysis results about Thalassinoides connectivity, and the relationships among BP, BS and SCS, as shown in FIGS. 9A-9C. The binary logistic regression results quantify the impact of each one of these variables for 1-meter-long samples. Results of the binary logistic regression, as listed in Tables 3A-3I, indicate that BP, BS and SCS are significant predictors for representing Thalassinoides connectivity in samples (P-value for all is <0.05). The B coefficients indicate that there is a negative impact of BS on Thalassinoides connectivity in samples, whereas there is a positive impact of BP and SCS on Thalassinoides connectivity in samples. Standardized β coefficients indicate that SCS has the highest impact on representation of Thalassinoides connectivity in the digital samples, followed by BP, and then BS, as shown in FIGS. 9A-9C. An important product of the logistic regression modelling is a probability equation 1 for samples to represent Thalassinoides connectivity in a 1-meter-long sample, as shown in FIG. 10. The equation is as follows:

$$Probability = 1 / ( 1 + exp ( - ( - 3.23 - 0.63 * BS + 0.091 * BP + 0.009 * SCS ) ) ) \qquad ( Equation 1 )$$

The aforementioned probability equation for the Thalassinoides connectivity can be used to assess the volume/SCS of 1-meter-long sample required to represent the connectivity of Thalassinoides, provided that the BP and BS are known. The internal validation of the results of the logistic regression modelling using the ROC curve for the predictive probability of the model with the training data revealed an area under the curve of 0.958 with P less than 0.05, as shown in FIG. 9B. When using the probability equation to predict Thalassinoides connectivity for the 108 samples of the external validation data set, the results revealed 85.29% specificity and 94.59 sensitivity. The accuracy of the probability model is about 91.67%. The ROC of the external validation using the 108 samples is 0.969, as shown in FIG. 9C.

TABLE 3A
Predictions and residuals (variable BC)

| Observation | | | | |
|---|---|---|---|---|
| Obs1 | 1 | 0 | 0.756 | 0.244 |
| Obs2 | 1 | 1 | 0.028 | 0.972 |
| Obs3 | 1 | 1 | 0.001 | 0.999 |
| Obs4 | 0 | 0 | 0.914 | 0.086 |
| Obs5 | 1 | 0 | 0.606 | 0.394 |
| Obs6 | 0 | 0 | 0.926 | 0.074 |
| Obs7 | 1 | 1 | 0.002 | 0.998 |
| Obs8 | 1 | 1 | 0.481 | 0.519 |
| Obs9 | 0 | 0 | 0.989 | 0.011 |
| Obs10 | 1 | 1 | 0.146 | 0.854 |
| Obs11 | 1 | 1 | 0.011 | 0.989 |
| Obs12 | 1 | 1 | 0.028 | 0.972 |
| Obs13 | 1 | 1 | 0.016 | 0.984 |
| Obs14 | 1 | 1 | 0.000 | 1.000 |
| Obs15 | 1 | 0 | 0.621 | 0.379 |
| Obs16 | 1 | 1 | 0.221 | 0.779 |
| Obs17 | 1 | 1 | 0.001 | 0.999 |
| Obs18 | 1 | 1 | 0.408 | 0.592 |
| Obs19 | 0 | 0 | 0.841 | 0.159 |
| Obs20 | 1 | 1 | 0.033 | 0.967 |
| Obs21 | 1 | 1 | 0.000 | 1.000 |
| Obs22 | 1 | 1 | 0.066 | 0.934 |
| Obs23 | 1 | 1 | 0.232 | 0.768 |
| Obs24 | 0 | 0 | 0.942 | 0.058 |
| Obs25 | 0 | 1 | 0.360 | 0.640 |
| Obs26 | 1 | 1 | 0.100 | 0.900 |
| Obs27 | 1 | 1 | 0.026 | 0.974 |
| Obs28 | 0 | 0 | 0.968 | 0.032 |
| Obs29 | 0 | 0 | 0.968 | 0.032 |
| Obs30 | 1 | 1 | 0.160 | 0.840 |
| Obs31 | 1 | 1 | 0.002 | 0.998 |
| Obs32 | 1 | 1 | 0.345 | 0.655 |
| Obs33 | 1 | 0 | 0.626 | 0.374 |
| Obs34 | 1 | 1 | 0.013 | 0.987 |
| Obs35 | 1 | 0 | 0.580 | 0.420 |
| Obs36 | 0 | 0 | 0.984 | 0.016 |
| Obs37 | 1 | 1 | 0.011 | 0.989 |
| Obs38 | 1 | 1 | 0.001 | 0.999 |
| Obs39 | 1 | 1 | 0.124 | 0.876 |
| Obs40 | 0 | 1 | 0.434 | 0.566 |
| Obs41 | 1 | 1 | 0.005 | 0.995 |
| Obs42 | 0 | 0 | 0.962 | 0.038 |
| Obs43 | 0 | 0 | 0.643 | 0.357 |
| Obs44 | 1 | 1 | 0.208 | 0.792 |
| Obs45 | 0 | 1 | 0.434 | 0.566 |
| Obs46 | 0 | 0 | 0.725 | 0.275 |
| Obs47 | 1 | 1 | 0.481 | 0.519 |
| Obs48 | 0 | 0 | 0.942 | 0.058 |
| Obs49 | 1 | 1 | 0.116 | 0.884 |
| Obs50 | 1 | 1 | 0.001 | 0.999 |
| Obs51 | 1 | 1 | 0.116 | 0.884 |
| Obs52 | 1 | 1 | 0.000 | 1.000 |
| Obs53 | 1 | 1 | 0.000 | 1.000 |
| Obs54 | 1 | 1 | 0.277 | 0.723 |
| Obs55 | 1 | 1 | 0.000 | 1.000 |
| Obs56 | 0 | 0 | 0.984 | 0.016 |
| Obs57 | 1 | 1 | 0.046 | 0.954 |
| Obs58 | 1 | 1 | 0.481 | 0.519 |
| Obs59 | 1 | 1 | 0.000 | 1.000 |
| Obs60 | 0 | 0 | 0.643 | 0.357 |
| Obs61 | 1 | 1 | 0.038 | 0.962 |
| Obs62 | 1 | 1 | 0.014 | 0.986 |
| Obs63 | 1 | 1 | 0.001 | 0.999 |
| Obs64 | 0 | 0 | 0.837 | 0.163 |
| Obs65 | 1 | 1 | 0.144 | 0.856 |
| Obs66 | 0 | 0 | 0.643 | 0.357 |
| Obs67 | 0 | 0 | 0.837 | 0.163 |
| Obs68 | 0 | 1 | 0.352 | 0.648 |
| Obs69 | 0 | 0 | 0.914 | 0.086 |
| Obs70 | 0 | 0 | 0.837 | 0.163 |
| Obs71 | 1 | 1 | 0.003 | 0.997 |
| Obs72 | 1 | 1 | 0.002 | 0.998 |
| Obs73 | 1 | 0 | 0.865 | 0.135 |
| Obs74 | 1 | 1 | 0.000 | 1.000 |
| Obs75 | 1 | 1 | 0.000 | 1.000 |
| Obs76 | 1 | 1 | 0.004 | 0.996 |
| Obs77 | 1 | 1 | 0.031 | 0.969 |
| Obs78 | 1 | 1 | 0.082 | 0.918 |
| Obs79 | 1 | 1 | 0.004 | 0.996 |
| Obs80 | 1 | 1 | 0.000 | 1.000 |
| Obs81 | 1 | 1 | 0.001 | 0.999 |
| Obs82 | 1 | 1 | 0.000 | 1.000 |
| Obs83 | 1 | 1 | 0.028 | 0.972 |
| Obs84 | 0 | 0 | 0.942 | 0.058 |
| Obs85 | 0 | 0 | 0.998 | 0.002 |
| Obs86 | 1 | 1 | 0.014 | 0.986 |
| Obs87 | 0 | 0 | 0.537 | 0.463 |
| Obs88 | 1 | 1 | 0.466 | 0.534 |
| Obs89 | 0 | 0 | 0.521 | 0.479 |
| Obs90 | 0 | 0 | 0.981 | 0.019 |
| Obs91 | 1 | 1 | 0.038 | 0.962 |
| Obs92 | 0 | 0 | 0.981 | 0.019 |
| Obs93 | 1 | 1 | 0.030 | 0.970 |
| Obs94 | 1 | 1 | 0.106 | 0.894 |
| Obs95 | 1 | 1 | 0.031 | 0.969 |
| Obs96 | 1 | 1 | 0.320 | 0.680 |
| Obs97 | 1 | 1 | 0.345 | 0.655 |
| Obs98 | 1 | 1 | 0.001 | 0.999 |
| Obs99 | 1 | 1 | 0.221 | 0.779 |
| Obs100 | 0 | 0 | 0.919 | 0.081 |
| Obs101 | 1 | 1 | 0.000 | 1.000 |
| Obs102 | 1 | 1 | 0.146 | 0.854 |
| Obs103 | 1 | 1 | 0.124 | 0.876 |
| Obs104 | 0 | 0 | 0.968 | 0.032 |
| Obs105 | 1 | 1 | 0.000 | 1.000 |
| Obs106 | 1 | 1 | 0.014 | 0.986 |
| Obs107 | 1 | 1 | 0.207 | 0.793 |
| Obs108 | 1 | 0 | 0.789 | 0.211 |
| Obs109 | 1 | 1 | 0.207 | 0.793 |
| Obs110 | 1 | 1 | 0.136 | 0.864 |
| Obs111 | 1 | 1 | 0.005 | 0.995 |
| Obs112 | 1 | 1 | 0.277 | 0.723 |
| Obs113 | 1 | 1 | 0.082 | 0.918 |
| Obs114 | 0 | 0 | 0.901 | 0.099 |
| Obs115 | 1 | 0 | 0.926 | 0.074 |
| Obs116 | 0 | 0 | 0.946 | 0.054 |
| Obs117 | 1 | 1 | 0.000 | 1.000 |
| Obs118 | 1 | 1 | 0.003 | 0.997 |
| Obs119 | 1 | 1 | 0.001 | 0.999 |
| Obs120 | 1 | 1 | 0.136 | 0.864 |
| Obs121 | 1 | 1 | 0.019 | 0.981 |
| Obs122 | 0 | 0 | 0.521 | 0.479 |
| Obs123 | 0 | 0 | 0.719 | 0.281 |
| Obs124 | 0 | 0 | 0.998 | 0.002 |
| Obs125 | 1 | 0 | 0.606 | 0.394 |
| Obs126 | 0 | 0 | 0.942 | 0.058 |
| Obs127 | 0 | 0 | 0.846 | 0.154 |
| Obs128 | 0 | 0 | 0.719 | 0.281 |
| Obs129 | 1 | 1 | 0.136 | 0.864 |
| Obs130 | 1 | 0 | 0.621 | 0.379 |
| Obs131 | 0 | 0 | 0.919 | 0.081 |
| Obs132 | 1 | 1 | 0.481 | 0.519 |
| Obs133 | 1 | 1 | 0.100 | 0.900 |
| Obs134 | 0 | 0 | 0.981 | 0.019 |
| Obs135 | 1 | 1 | 0.054 | 0.946 |
| Obs136 | 1 | 1 | 0.011 | 0.989 |
| Obs137 | 0 | 0 | 0.997 | 0.003 |
| Obs138 | 1 | 1 | 0.000 | 1.000 |
| Obs139 | 1 | 1 | 0.000 | 1.000 |
| Obs140 | 1 | 1 | 0.116 | 0.884 |
| Obs141 | 0 | 0 | 0.946 | 0.054 |
| Obs142 | 1 | 0 | 0.901 | 0.099 |
| Obs143 | 1 | 1 | 0.004 | 0.996 |
| Obs144 | 0 | 0 | 0.981 | 0.019 |
| Obs145 | 1 | 1 | 0.016 | 0.984 |
| Obs146 | 1 | 1 | 0.051 | 0.949 |
| Obs147 | 1 | 1 | 0.136 | 0.864 |
| Obs148 | 1 | 1 | 0.005 | 0.995 |
| Obs149 | 0 | 0 | 0.963 | 0.037 |
| Obs150 | 1 | 1 | 0.000 | 1.000 |
| Obs151 | 1 | 1 | 0.033 | 0.967 |
| Obs152 | 1 | 1 | 0.000 | 1.000 |
| Obs153 | 0 | 1 | 0.160 | 0.840 |
| Obs154 | 1 | 1 | 0.033 | 0.967 |
| Obs155 | 1 | 1 | 0.003 | 0.997 |
| Obs156 | 1 | 1 | 0.004 | 0.996 |
| Obs157 | 1 | 1 | 0.002 | 0.998 |
| Obs158 | 1 | 1 | 0.005 | 0.995 |
| Obs159 | 1 | 1 | 0.000 | 1.000 |
| Obs160 | 0 | 0 | 0.865 | 0.135 |
| Obs161 | 1 | 1 | 0.002 | 0.998 |
| Obs162 | 0 | 0 | 0.989 | 0.011 |
| Obs163 | 1 | 1 | 0.017 | 0.983 |
| Obs164 | 1 | 1 | 0.005 | 0.995 |
| Obs165 | 1 | 1 | 0.213 | 0.787 |
| Obs166 | 0 | 0 | 0.650 | 0.350 |
| Obs167 | 0 | 0 | 0.719 | 0.281 |
| Obs168 | 1 | 1 | 0.106 | 0.894 |
| Obs169 | 0 | 0 | 0.998 | 0.002 |
| Obs170 | 1 | 1 | 0.051 | 0.949 |
| Obs171 | 0 | 0 | 0.650 | 0.350 |
| Obs172 | 1 | 1 | 0.000 | 1.000 |
| Obs173 | 0 | 0 | 0.580 | 0.420 |
| Obs174 | 1 | 1 | 0.046 | 0.954 |
| Obs175 | 1 | 1 | 0.001 | 0.999 |
| Obs176 | 1 | 1 | 0.000 | 1.000 |
| Obs177 | 1 | 1 | 0.001 | 0.999 |
| Obs178 | 1 | 1 | 0.100 | 0.900 |
| Obs179 | 1 | 1 | 0.013 | 0.987 |
| Obs180 | 0 | 0 | 0.989 | 0.011 |
| Obs181 | 1 | 1 | 0.352 | 0.648 |
| Obs182 | 1 | 1 | 0.014 | 0.986 |
| Obs183 | 1 | 1 | 0.066 | 0.934 |
| Obs184 | 1 | 1 | 0.054 | 0.946 |
| Obs185 | 1 | 1 | 0.001 | 0.999 |
| Obs186 | 1 | 1 | 0.005 | 0.995 |
| Obs187 | 1 | 1 | 0.030 | 0.970 |
| Obs188 | 1 | 1 | 0.011 | 0.989 |
| Obs189 | 1 | 1 | 0.144 | 0.856 |
| Obs190 | 1 | 1 | 0.116 | 0.884 |
| Obs191 | 1 | 0 | 0.837 | 0.163 |
| Obs192 | 0 | 1 | 0.116 | 0.884 |
| Obs193 | 0 | 0 | 0.719 | 0.281 |
| Obs194 | 0 | 0 | 0.984 | 0.016 |
| Obs195 | 1 | 1 | 0.000 | 1.000 |
| Obs196 | 0 | 0 | 0.981 | 0.019 |
| Obs197 | 1 | 1 | 0.073 | 0.927 |
| Obs198 | 1 | 1 | 0.208 | 0.792 |
| Obs199 | 1 | 1 | 0.026 | 0.974 |
| Obs200 | 1 | 1 | 0.000 | 1.000 |
| Obs201 | 0 | 0 | 0.926 | 0.074 |
| Obs202 | 1 | 1 | 0.017 | 0.983 |
| Obs203 | 0 | 0 | 0.521 | 0.479 |
| Obs204 | 1 | 1 | 0.003 | 0.997 |
| Obs205 | 1 | 1 | 0.232 | 0.768 |
| Obs206 | 1 | 1 | 0.160 | 0.840 |
| Obs207 | 1 | 1 | 0.001 | 0.999 |
| Obs208 | 0 | 0 | 0.984 | 0.016 |
| Obs209 | 1 | 1 | 0.030 | 0.970 |
| Obs210 | 0 | 0 | 0.919 | 0.081 |
| Obs211 | 0 | 0 | 0.537 | 0.463 |
| Obs212 | 1 | 1 | 0.005 | 0.995 |
| Obs213 | 1 | 1 | 0.054 | 0.946 |
| Obs214 | 1 | 1 | 0.160 | 0.840 |
| Obs215 | 0 | 0 | 0.756 | 0.244 |
| Obs216 | 1 | 1 | 0.124 | 0.876 |
| Obs217 | 0 | 1 | 0.434 | 0.566 |
| Obs218 | 1 | 1 | 0.232 | 0.768 |
| Obs219 | 0 | 0 | 0.789 | 0.211 |
| Obs220 | 1 | 1 | 0.360 | 0.640 |
| Obs221 | 0 | 0 | 0.882 | 0.118 |
| Obs222 | 0 | 0 | 0.865 | 0.135 |
| Obs223 | 1 | 1 | 0.232 | 0.768 |
| Obs224 | 1 | 1 | 0.005 | 0.995 |
| Obs225 | 1 | 1 | 0.000 | 1.000 |
| Obs226 | 1 | 1 | 0.136 | 0.864 |
| Obs227 | 0 | 0 | 0.926 | 0.074 |
| Obs228 | 0 | 0 | 0.725 | 0.275 |
| Obs229 | 1 | 1 | 0.207 | 0.793 |
| Obs230 | 1 | 1 | 0.031 | 0.969 |
| Obs231 | 0 | 0 | 0.521 | 0.479 |
| Obs232 | 0 | 0 | 0.537 | 0.463 |
| Obs233 | 0 | 0 | 0.865 | 0.135 |
| Obs234 | 1 | 0 | 0.621 | 0.379 |
| Obs235 | 1 | 1 | 0.005 | 0.995 |
| Obs236 | 0 | 0 | 0.895 | 0.105 |
| Obs237 | 0 | 0 | 0.962 | 0.038 |
| Obs238 | 1 | 1 | 0.003 | 0.997 |
| Obs239 | 0 | 0 | 0.914 | 0.086 |
| Obs240 | 0 | 0 | 0.537 | 0.463 |
| Obs241 | 1 | 0 | 0.926 | 0.074 |
| Obs242 | 1 | 1 | 0.051 | 0.949 |
| Obs243 | 1 | 1 | 0.073 | 0.927 |
| Obs244 | 1 | 0 | 0.606 | 0.394 |
| Obs245 | 1 | 1 | 0.003 | 0.997 |
| Obs246 | 1 | 1 | 0.000 | 1.000 |
| Obs247 | 1 | 1 | 0.000 | 1.000 |
| Obs248 | 1 | 1 | 0.051 | 0.949 |
| Obs249 | 0 | 1 | 0.213 | 0.787 |
| Obs250 | 1 | 1 | 0.221 | 0.779 |
| Obs251 | 0 | 0 | 0.643 | 0.357 |
| Obs252 | 0 | 0 | 0.650 | 0.350 |
| Obs253 | 0 | 0 | 0.580 | 0.420 |
| Obs254 | 1 | 1 | 0.046 | 0.954 |
| Obs255 | 0 | 0 | 0.621 | 0.379 |
| Obs256 | 0 | 1 | 0.011 | 0.989 |
| Obs257 | 1 | 1 | 0.016 | 0.984 |
| Obs258 | 0 | 0 | 0.946 | 0.054 |
| Obs259 | 1 | 1 | 0.277 | 0.723 |
| Obs260 | 1 | 1 | 0.002 | 0.998 |
| Obs261 | 0 | 0 | 0.989 | 0.011 |
| Obs262 | 1 | 1 | 0.005 | 0.995 |
| Obs263 | 0 | 0 | 0.580 | 0.420 |
| Obs264 | 1 | 1 | 0.001 | 0.999 |
| Obs265 | 0 | 0 | 0.882 | 0.118 |
| Obs266 | 0 | 0 | 0.882 | 0.118 |
| Obs267 | 0 | 0 | 0.901 | 0.099 |
| Obs268 | 1 | 1 | 0.345 | 0.655 |
| Obs269 | 0 | 1 | 0.106 | 0.894 |
| Obs270 | 0 | 1 | 0.360 | 0.640 |
| Obs271 | 0 | 0 | 0.968 | 0.032 |
| Obs272 | 1 | 1 | 0.016 | 0.984 |
| Obs273 | 1 | 1 | 0.000 | 1.000 |
| Obs274 | 1 | 1 | 0.221 | 0.779 |
| Obs275 | 0 | 0 | 0.841 | 0.159 |
| Obs276 | 1 | 1 | 0.466 | 0.534 |
| Obs277 | 1 | 1 | 0.001 | 0.999 |
| Obs278 | 0 | 1 | 0.352 | 0.648 |
| Obs279 | 1 | 1 | 0.002 | 0.998 |
| Obs280 | 1 | 1 | 0.013 | 0.987 |
| Obs281 | 0 | 0 | 0.919 | 0.081 |
| Obs282 | 0 | 0 | 0.968 | 0.032 |
| Obs283 | 0 | 0 | 0.841 | 0.159 |
| Obs284 | 0 | 1 | 0.320 | 0.680 |
| Obs285 | 1 | 1 | 0.016 | 0.984 |
| Obs286 | 1 | 1 | 0.003 | 0.997 |
| Obs287 | 0 | 0 | 0.946 | 0.054 |
| Obs288 | 1 | 1 | 0.002 | 0.998 |
| Obs289 | 1 | 1 | 0.031 | 0.969 |
| Obs290 | 1 | 0 | 0.756 | 0.244 |
| Obs291 | 1 | 1 | 0.003 | 0.997 |
| Obs292 | 1 | 1 | 0.320 | 0.680 |
| Obs293 | 1 | 1 | 0.073 | 0.927 |
| Obs294 | 0 | 0 | 0.895 | 0.105 |
| Obs295 | 1 | 1 | 0.001 | 0.999 |
| Obs296 | 1 | 1 | 0.007 | 0.993 |
| Obs297 | 1 | 1 | 0.026 | 0.974 |
| Obs298 | 1 | 1 | 0.033 | 0.967 |
| Obs299 | 1 | 1 | 0.008 | 0.992 |
| Obs300 | 1 | 1 | 0.000 | 1.000 |
| Obs301 | 0 | 0 | 0.561 | 0.439 |
| Obs302 | 0 | 0 | 0.846 | 0.154 |
| Obs303 | 0 | 0 | 0.719 | 0.281 |
| Obs304 | 0 | 1 | 0.408 | 0.592 |
| Obs305 | 0 | 1 | 0.208 | 0.792 |
| Obs306 | 1 | 1 | 0.003 | 0.997 |
| Obs307 | 1 | 1 | 0.016 | 0.984 |
| Obs308 | 1 | 1 | 0.001 | 0.999 |
| Obs309 | 1 | 1 | 0.000 | 1.000 |
| Obs310 | 1 | 1 | 0.003 | 0.997 |
| Obs311 | 0 | 0 | 0.561 | 0.439 |
| Obs312 | 0 | 0 | 0.901 | 0.099 |
| Obs313 | 1 | 1 | 0.054 | 0.946 |
| Obs314 | 1 | 1 | 0.000 | 1.000 |
| Obs315 | 0 | 0 | 0.926 | 0.074 |
| Obs316 | 0 | 1 | 0.156 | 0.844 |
| Obs317 | 1 | 1 | 0.051 | 0.949 |
| Obs318 | 1 | 1 | 0.000 | 1.000 |
| Obs319 | 0 | 0 | 0.580 | 0.420 |
| Obs320 | 0 | 0 | 0.914 | 0.086 |
| Obs321 | 1 | 1 | 0.408 | 0.592 |
| Obs322 | 1 | 1 | 0.082 | 0.918 |
| Obs323 | 1 | 1 | 0.004 | 0.996 |
| Obs324 | 1 | 1 | 0.000 | 1.000 |
| Obs325 | 1 | 1 | 0.004 | 0.996 |
| Obs326 | 0 | 1 | 0.136 | 0.864 |
| Obs327 | 0 | 0 | 0.521 | 0.479 |
| Obs328 | 1 | 1 | 0.000 | 1.000 |
| Obs329 | 1 | 1 | 0.345 | 0.655 |
| Obs330 | 1 | 1 | 0.008 | 0.992 |
| Obs331 | 1 | 1 | 0.054 | 0.946 |
| Obs332 | 1 | 1 | 0.016 | 0.984 |
| Obs333 | 1 | 1 | 0.073 | 0.927 |
| Obs334 | 0 | 1 | 0.136 | 0.864 |
| Obs335 | 1 | 1 | 0.016 | 0.984 |
| Obs336 | 1 | 0 | 0.626 | 0.374 |
| Obs337 | 0 | 0 | 0.962 | 0.038 |
| Obs338 | 0 | 0 | 0.901 | 0.099 |
| Obs339 | 1 | 1 | 0.232 | 0.768 |
| Obs340 | 0 | 0 | 0.650 | 0.350 |
| Obs341 | 1 | 1 | 0.156 | 0.844 |
| Obs342 | 0 | 0 | 0.789 | 0.211 |
| Obs343 | 1 | 1 | 0.051 | 0.949 |
| Obs344 | 1 | 1 | 0.003 | 0.997 |
| Obs345 | 1 | 1 | 0.002 | 0.998 |
| Obs346 | 1 | 1 | 0.144 | 0.856 |
| Obs347 | 0 | 0 | 0.650 | 0.350 |
| Obs348 | 0 | 0 | 0.719 | 0.281 |
| Obs349 | 1 | 1 | 0.156 | 0.844 |
| Obs350 | 0 | 1 | 0.116 | 0.884 |
| Obs351 | 1 | 1 | 0.002 | 0.998 |
| Obs352 | 1 | 1 | 0.434 | 0.566 |
| Obs353 | 1 | 1 | 0.207 | 0.793 |
| Obs354 | 0 | 0 | 0.561 | 0.439 |
| Obs355 | 1 | 0 | 0.882 | 0.118 |
| Obs356 | 1 | 1 | 0.030 | 0.970 |
| Obs357 | 1 | 1 | 0.001 | 0.999 |
| Obs358 | 1 | 1 | 0.207 | 0.793 |
| Obs359 | 0 | 1 | 0.156 | 0.844 |
| Obs360 | 1 | 1 | 0.002 | 0.998 |
| Obs361 | 1 | 1 | 0.007 | 0.993 |
| Obs362 | 1 | 1 | 0.007 | 0.993 |
| Obs363 | 1 | 1 | 0.001 | 0.999 |
| Obs364 | 1 | 1 | 0.028 | 0.972 |
| Obs365 | 1 | 1 | 0.005 | 0.995 |
| Obs366 | 0 | 0 | 0.919 | 0.081 |
| Obs367 | 1 | 1 | 0.005 | 0.995 |
| Obs368 | 1 | 1 | 0.136 | 0.864 |
| Obs369 | 1 | 1 | 0.019 | 0.981 |
| Obs370 | 1 | 1 | 0.000 | 1.000 |
| Obs371 | 0 | 0 | 0.989 | 0.011 |
| Obs372 | 1 | 1 | 0.005 | 0.995 |
| Obs373 | 0 | 0 | 0.537 | 0.463 |
| Obs374 | 0 | 0 | 0.895 | 0.105 |
| Obs375 | 1 | 1 | 0.277 | 0.723 |
| Obs376 | 0 | 0 | 0.984 | 0.016 |
| Obs377 | 1 | 1 | 0.106 | 0.894 |
| Obs378 | 1 | 1 | 0.208 | 0.792 |
| Obs379 | 1 | 0 | 0.719 | 0.281 |
| Obs380 | 1 | 1 | 0.004 | 0.996 |
| Obs381 | 1 | 1 | 0.066 | 0.934 |
| Obs382 | 1 | 1 | 0.100 | 0.900 |
| Obs383 | 0 | 0 | 0.725 | 0.275 |
| Obs384 | 1 | 1 | 0.116 | 0.884 |
| Obs385 | 1 | 1 | 0.116 | 0.884 |
| Obs386 | 1 | 1 | 0.003 | 0.997 |
| Obs387 | 1 | 1 | 0.004 | 0.996 |
| Obs388 | 1 | 1 | 0.001 | 0.999 |
| Obs389 | 1 | 1 | 0.000 | 1.000 |
| Obs390 | 1 | 1 | 0.026 | 0.974 |
| Obs391 | 0 | 0 | 0.606 | 0.394 |
| Obs392 | 1 | 1 | 0.000 | 1.000 |
| Obs393 | 1 | 1 | 0.116 | 0.884 |
| Obs394 | 1 | 1 | 0.434 | 0.566 |
| Obs395 | 1 | 1 | 0.073 | 0.927 |
| Obs396 | 0 | 0 | 0.963 | 0.037 |
| Obs397 | 1 | 0 | 0.837 | 0.163 |
| Obs398 | 1 | 1 | 0.008 | 0.992 |
| Obs399 | 1 | 1 | 0.124 | 0.876 |
| Obs400 | 0 | 0 | 0.895 | 0.105 |
| Obs401 | 1 | 1 | 0.073 | 0.927 |
| Obs402 | 0 | 1 | 0.208 | 0.792 |
| Obs403 | 1 | 0 | 0.606 | 0.394 |
| Obs404 | 1 | 1 | 0.466 | 0.534 |
| Obs405 | 1 | 1 | 0.082 | 0.918 |
| Obs406 | 1 | 1 | 0.003 | 0.997 |
| Obs407 | 1 | 1 | 0.001 | 0.999 |
| Obs408 | 1 | 0 | 0.865 | 0.135 |
| Obs409 | 1 | 1 | 0.026 | 0.974 |
| Obs410 | 1 | 1 | 0.030 | 0.970 |
| Obs411 | 1 | 1 | 0.000 | 1.000 |
| Obs412 | 1 | 1 | 0.008 | 0.992 |
| Obs413 | 1 | 1 | 0.000 | 1.000 |
| Obs414 | 1 | 1 | 0.000 | 1.000 |
| Obs415 | 1 | 1 | 0.000 | 1.000 |
| Obs416 | 0 | 1 | 0.213 | 0.787 |
| Obs417 | 1 | 1 | 0.481 | 0.519 |
| Obs418 | 1 | 1 | 0.031 | 0.969 |
| Obs419 | 0 | 0 | 0.997 | 0.003 |
| Obs420 | 0 | 1 | 0.213 | 0.787 |
| Obs421 | 1 | 1 | 0.002 | 0.998 |
| Obs422 | 0 | 0 | 0.626 | 0.374 |
| Obs423 | 1 | 1 | 0.136 | 0.864 |
| Obs424 | 1 | 1 | 0.073 | 0.927 |
| Obs425 | 1 | 1 | 0.001 | 0.999 |
| Obs426 | 1 | 0 | 0.719 | 0.281 |
| Obs427 | 0 | 0 | 0.895 | 0.105 |
| Obs428 | 0 | 0 | 0.841 | 0.159 |
| Obs429 | 1 | 1 | 0.017 | 0.983 |
| Obs430 | 1 | 1 | 0.000 | 1.000 |
| Obs431 | 1 | 1 | 0.051 | 0.949 |
| Obs432 | 1 | 1 | 0.003 | 0.997 |
| Obs433 | 0 | 0 | 0.882 | 0.118 |
| Obs434 | 0 | 0 | 0.725 | 0.275 |
| Obs435 | 1 | 1 | 0.038 | 0.962 |
| Obs436 | 1 | 1 | 0.000 | 1.000 |
| Obs437 | 1 | 1 | 0.038 | 0.962 |
| Obs438 | 1 | 1 | 0.100 | 0.900 |
| Obs439 | 0 | 0 | 0.719 | 0.281 |
| Obs440 | 0 | 1 | 0.116 | 0.884 |
| Obs441 | 0 | 0 | 0.942 | 0.058 |
| Obs442 | 1 | 1 | 0.066 | 0.934 |
| Obs443 | 1 | 1 | 0.007 | 0.993 |
| Obs444 | 1 | 1 | 0.320 | 0.680 |
| Obs445 | 1 | 1 | 0.345 | 0.655 |
| Obs446 | 0 | 1 | 0.360 | 0.640 |
| Obs447 | 1 | 1 | 0.007 | 0.993 |
| Obs448 | 1 | 1 | 0.221 | 0.779 |
| Obs449 | 1 | 1 | 0.144 | 0.856 |
| Obs450 | 0 | 0 | 0.962 | 0.038 |
| Obs451 | 1 | 1 | 0.146 | 0.854 |
| Obs452 | 0 | 0 | 0.997 | 0.003 |
| Obs453 | 0 | 1 | 0.352 | 0.648 |
| Obs454 | 1 | 1 | 0.013 | 0.987 |
| Obs455 | 0 | 0 | 0.963 | 0.037 |
| Obs456 | 1 | 1 | 0.156 | 0.844 |
| Obs457 | 1 | 1 | 0.002 | 0.998 |
| Obs458 | 1 | 1 | 0.038 | 0.962 |
| Obs459 | 1 | 1 | 0.000 | 1.000 |
| Obs460 | 1 | 1 | 0.001 | 0.999 |
| Obs461 | 1 | 1 | 0.005 | 0.995 |
| Obs462 | 0 | 0 | 0.963 | 0.037 |
| Obs463 | 1 | 1 | 0.017 | 0.983 |
| Obs464 | 1 | 1 | 0.002 | 0.998 |
| Obs465 | 1 | 0 | 0.756 | 0.244 |
| Obs466 | 0 | 0 | 0.725 | 0.275 |
| Obs467 | 1 | 1 | 0.019 | 0.981 |
| Obs468 | 0 | 0 | 0.914 | 0.086 |
| Obs469 | 1 | 1 | 0.028 | 0.972 |
| Obs470 | 1 | 1 | 0.051 | 0.949 |
| Obs471 | 0 | 0 | 0.789 | 0.211 |
| Obs472 | 1 | 1 | 0.066 | 0.934 |
| Obs473 | 1 | 1 | 0.051 | 0.949 |
| Obs474 | 1 | 0 | 0.756 | 0.244 |
| Obs475 | 0 | 0 | 0.643 | 0.357 |
| Obs476 | 1 | 1 | 0.073 | 0.927 |
| Obs477 | 1 | 1 | 0.124 | 0.876 |
| Obs478 | 1 | 1 | 0.003 | 0.997 |
| Obs479 | 1 | 1 | 0.146 | 0.854 |
| Obs480 | 1 | 1 | 0.466 | 0.534 |
| Obs481 | 1 | 1 | 0.136 | 0.864 |
| Obs482 | 1 | 1 | 0.000 | 1.000 |
| Obs483 | 1 | 1 | 0.005 | 0.995 |
| Obs484 | 0 | 0 | 0.997 | 0.003 |
| Obs485 | 1 | 1 | 0.008 | 0.992 |
| Obs486 | 1 | 1 | 0.017 | 0.983 |
| Obs487 | 1 | 1 | 0.320 | 0.680 |
| Obs488 | 1 | 1 | 0.046 | 0.954 |
| Obs489 | 0 | 1 | 0.073 | 0.927 |
| Obs490 | 1 | 1 | 0.002 | 0.998 |
| Obs491 | 1 | 1 | 0.046 | 0.954 |
| Obs492 | 1 | 1 | 0.082 | 0.918 |
| Obs493 | 1 | 1 | 0.004 | 0.996 |
| Obs494 | 0 | 0 | 0.998 | 0.002 |
| Obs495 | 1 | 1 | 0.001 | 0.999 |
| Obs496 | 0 | 0 | 0.998 | 0.002 |
| Obs497 | 1 | 0 | 0.621 | 0.379 |
| Obs498 | 0 | 0 | 0.841 | 0.159 |
| Obs499 | 0 | 0 | 0.789 | 0.211 |
| Obs500 | 0 | 0 | 0.626 | 0.374 |
| Obs501 | 1 | 1 | 0.016 | 0.984 |
| Obs502 | 1 | 1 | 0.073 | 0.927 |
| Obs503 | 1 | 1 | 0.014 | 0.986 |
| Obs504 | 1 | 1 | 0.408 | 0.592 |
| Obs505 | 1 | 1 | 0.019 | 0.981 |
| Obs506 | 1 | 1 | 0.000 | 1.000 |
| Obs507 | 1 | 1 | 0.033 | 0.967 |
| Obs508 | 0 | 0 | 0.626 | 0.374 |
| Obs509 | 0 | 0 | 0.997 | 0.003 |
| Obs510 | 1 | 1 | 0.013 | 0.987 |
| Obs511 | 1 | 1 | 0.160 | 0.840 |
| Obs512 | 1 | 1 | 0.000 | 1.000 |
| Obs513 | 1 | 1 | 0.000 | 1.000 |
| Obs514 | 1 | 1 | 0.408 | 0.592 |
| Obs515 | 0 | 0 | 0.846 | 0.154 |
| Obs516 | 0 | 0 | 0.946 | 0.054 |
| Obs517 | 0 | 1 | 0.352 | 0.648 |
| Obs518 | 0 | 0 | 0.963 | 0.037 |
| Obs519 | 0 | 1 | 0.213 | 0.787 |
| Obs520 | 1 | 1 | 0.000 | 1.000 |
| Obs521 | 0 | 0 | 0.846 | 0.154 |
| Obs522 | 1 | 0 | 0.561 | 0.439 |
| Obs523 | 1 | 1 | 0.466 | 0.534 |
| Obs524 | 1 | 1 | 0.000 | 1.000 |
| Obs525 | 1 | 1 | 0.004 | 0.996 |
| Obs526 | 0 | 1 | 0.106 | 0.894 |
| Obs527 | 0 | 0 | 0.846 | 0.154 |
| Obs528 | 1 | 1 | 0.001 | 0.999 |
| Obs529 | 1 | 1 | 0.016 | 0.984 |
| Obs530 | 1 | 1 | 0.277 | 0.723 |
| Obs531 | 0 | 0 | 0.561 | 0.439 |
| Obs532 | 0 | 0 | 0.719 | 0.281 |
| Obs533 | 1 | 1 | 0.144 | 0.856 |
| Obs534 | 1 | 1 | 0.001 | 0.999 |
| Obs535 | 1 | 1 | 0.000 | 1.000 |
| Obs536 | 1 | 1 | 0.019 | 0.981 |
| Obs537 | 1 | 1 | 0.146 | 0.854 |
| Obs538 | 1 | 1 | 0.051 | 0.949 |
| Obs539 | 1 | 1 | 0.000 | 1.000 |
| Obs540 | 1 | 1 | 0.360 | 0.640 |

TABLE 3B
Goodness of fit statistics (variable BC)

| Statistic | Independent | Full |
|---|---|---|
| Observations | 432 | 432 |
| Sum of weights | 432.000 | 432.000 |
| DF | 431 | 428 |
| −2 Log(Likelihood) | 542.755 | 240.107 |
| R²(McFadden) | 0.000 | 0.558 |
| R²(Cox and Snell) | 0.000 | 0.504 |
| R²(Nagelkerke) | 0.000 | 0.701 |
| AIC | 544.755 | 248.107 |
| SBC | 548.823 | 264.381 |
| Iterations | 0 | 6 |

TABLE 3C
Test of null hypothesis Pr(BC = 1) = 0.678

| Statistic | DF | Chi-square | Pr > Chi² |
|---|---|---|---|
| −2 Log(Likelihood) | 3 | 302.647 | <0.0001 |
| Score | 3 | 204.837 | <0.0001 |
| Wald | 3 | 94.786 | <0.0001 |

TABLE 3D
Type-II analysis (variable BC)

| | | Chi-square | | Chi-square | |
|---|---|---|---|---|---|
| Source | DF | (Wald) | Pr > Wald | (LR) | Pr > LR |
| BS | 1 | 49.620 | <0.0001 | 72.563 | <0.0001 |
| BP | 1 | 68.961 | <0.0001 | 117.499 | <0.0001 |
| SCS | 1 | 75.014 | <0.0001 | 192.392 | <0.0001 |

TABLE 3E
Hosmer-Lemeshow test (variable BC0)

| Statistic | Chi-square | DF | Pr > Chi² |
|---|---|---|---|
| Hosmer-Lemeshow Statistic | 8.730 | 8 | 0.366 |

TABLE 3F
Model parameters (variable BC)

| | | | | | | | | Odd | Odds |
|---|---|---|---|---|---|---|---|---|---|
| | | | | | Wald | Wald | | ratio | ratio |
| | | | Wald | | Lower | Upper | | lower | Upper |
| | | Standard | Chi- | Pr > | bound | bound | Odds | bounds | bound |
| Source | Value | error | Square | Chi² | (95%) | (95%) | ratio | (95%) | (95%) |
| Intercept | −3.235 | 0.594 | 29.614 | <0.0001 | −4.400 | −2.070 | | | |
| BS | −0.633 | 0.090 | 49.620 | <0.0001 | −0.809 | −0.457 | 0.531 | 0.445 | 0.633 |
| BP | 0.091 | 0.011 | 68.961 | <0.0001 | 0.070 | 0.113 | 1.095 | 1.072 | 1.119 |
| SCS | 0.010 | 0.001 | 75.014 | <0.0001 | 0.007 | 0.012 | 1.010 | 1.007 | 1.012 |

TABLE 3G
Standardized coefficients (variable BC)

| | | | | | Wald | Wald |
|---|---|---|---|---|---|---|
| | | | Wald | | Lower | Upper |
| | | Standard | Chi- | Pr > | bound | bound |
| Source | Value | error | Square | Chi² | (95%) | (95%) |
| BS | −0.759 | 0.108 | 49.620 | <0.0001 | −0.970 | −0.548 |
| BP | 1.031 | 0.124 | 68.961 | <0.0001 | 0.787 | 1.274 |
| SCS | 1.613 | 0.186 | 75.014 | <0.0001 | 1.248 | 1.978 |

TABLE 3H
Classification for the training samples (variable BC)

| | to | | | |
|---|---|---|---|---|
| from | 0 | 1 | Total | % Correct |
| 0 | 116 | 23 | 139 | 83.45% |
| 1 | 23 | 270 | 293 | 92.15% |
| Total | 139 | 293 | 432 | 89.35% |

TABLE 3I
Classification for the validation sample (variable BC)

| | to | | | |
|---|---|---|---|---|
| from | 0 | 1 | Total | % Correct |
| 0 | 27 | 7 | 34 | 79.41% |
| 1 | 4 | 70 | 74 | 94.59% |
| Total | 31 | 77 | 108 | 89.81% |

### Example 11: Sampling Design Implications

The digital samples generated in the present disclosure are limited by computational constraints. The focus was to get the smallest SCS as close as possible to core-plug and rotary side wall core dimensions; hence a SCS of about 25 cm² was achieved. This is equivalent to a cylindrical sample with 2.2-inch diameter, close to the 1 to 1.5 inches of typical core plugs. Similarly, the 100 cm² SCS has an area comparable to a cylinder with 4.4-inch diameter. This is similar in SCS to 4-inch drill core. However, the digital samples of the present disclosure have a 1-meter-length which is substantially longer than the samples used in the laboratory to measure permeability. The typical length of laboratory samples depends on their diameter. For example, core plugs typically are only 1 to 2 inches long. Whole core (full diameter) analyses typically are somewhere in the range of about 4 to 10-inches. Results of the experimentation conducted in the present disclosure allows for analysis of the controls on the shorter-than-1 m-samples. In order to perform the said procedure, the results from 25 cm² and 100 cm² SCS runs were analysed as proxies for core plugs and 4-inch core, respectively, documented in Table 5A and Table 6A. Out of these 180 samples (90 samples for each cross section), 67 samples have LCBV that span the entire 1 m length of the digital samples, and the rest 113 samples have LCBV with lengths less than 1 m. For the 67 samples with 1 m-long LCBVs, the combination of SCS, BP and BS yield results that assure that any length of vertical sample will represent the vertical connectivity of the burrows. For the other 113 samples, however, the combination of SCS, BP and BS do not yield LCBVs that span the entire 1-meter-length, and thus, shorter samples cannot be guaranteed of representing the vertical connectivity of the burrows. For these, the probability of a particular 1- to 10-inch-long sample falling within the LCBV depends on the SCS, BS, BP, and the length of the sample segment. The probability of those sample segments falling fully within the LCBV, and thus correctly representing permeability, can be calculated as follows. Results provided the length of the LCBV for each combination of SCS, BS, and BP, the results are provided in Table 4A. Given samples of specified length (lS), a number of samples can be calculated (#Sa) that may fall within the length of the LCBV as represented in equation 2. Since it is unlikely that the two samples at the top and bottom of the LCBV lie fully within the LCBV, the integer of 2 is subtracted from the product of equation 2 to produce the number of samples of a specified length that would fall fully within the length of the LCBV, as represented by equation 3. Since the columns have 1-m length, the total number of samples possible (#St), of a specified length (lS) is produced by dividing 100 by the length of the subsample (lS in cm), as represented in equation 4. The probability of having the LCBV represented in the subsamples may be calculated by dividing the product of equation 3 by the product of equation 4 as represented in equation 5.

$$\# Sa = \frac{lLCBV}{lS} \qquad ( Equation 2 )$$

$$\# S β = \frac{lLCBV}{lS} - 2 \qquad ( Equation 3 )$$

$$\# St = \frac{100}{lS} \qquad ( Equation 4 )$$

$$pLCBV = \frac{\# Sb}{\# St} \qquad ( Equation 5 )$$

Where, #Sa is total samples possible in LCBV, #Sb is total samples possible fully in LCBV, #St is total samples possible of specified length, lLCBV is length of the LCBV, lS is length of the sample, and pLCBV is the probability of having the LCBV represented in a sample.

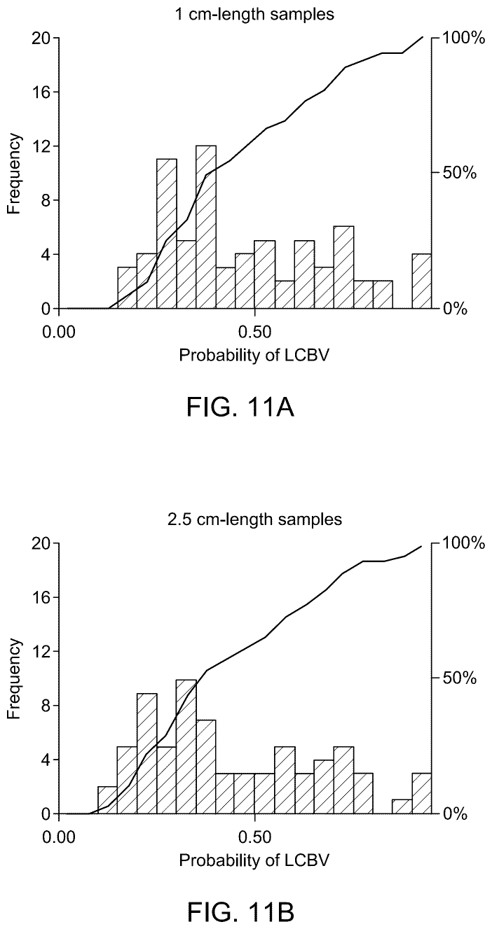

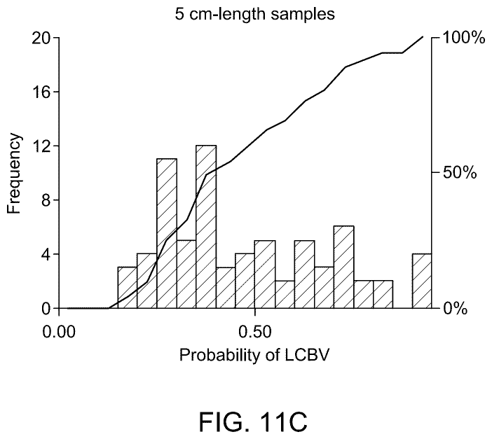

Further, a total of 71 digital samples has LCBV less than 100 cm in the digital samples meant to simulate core plug sampling, with SCS of 25 cm² (90 samples), as documented in Table 4A. Applying the above equations to 71 digital samples using the lS of 5 cm, calculates the probability of a 5 cm-long sample sampling the LCBV (represented by pLCBV). As shown in FIG. 11C, only 28% of digital samples have 0.5 or greater pLCBV. The same analysis was carried out for shorter sample lengths (1 and 2.5 cm). For the 1 cm length samples, as shown in FIG. 11A, only 37% of digital samples yield 0.5 or greater pLCBV. For the 2.5 cm length samples, as shown in FIG. 11B, 34% of digital samples yield 0.5 or greater pLCBV.

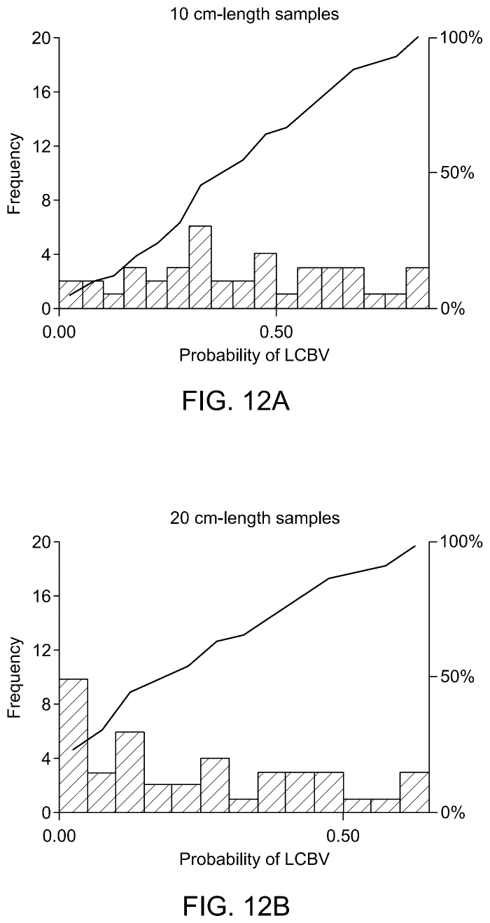

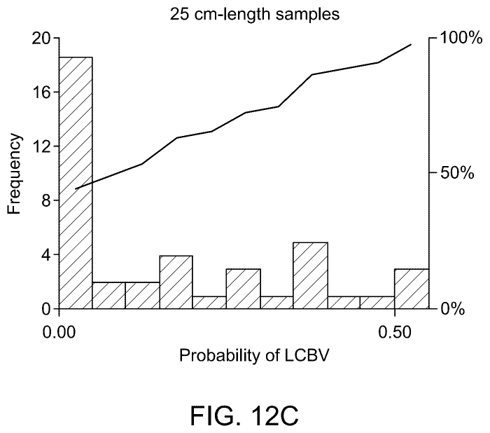

Similarly, for the digital samples meant to simulate whole core, with SCS of 100 cm² (90 samples), as documented in Table 6A, 42 of the digital samples have LCBV of less than 100 cm. Applying the above specified equations to the 42 samples, with lS of 10 cm, 20 cm, and 25 cm to simulate various lengths of whole-core samples may be simulated, as shown in FIGS. 12A-12C. The calculated pLCBV of these samples ranges from 0% to 78% for the 10 cm length, from 0% to 58% for the 20 cm length, and from 0% to 48% for the 25 cm length. As shown in FIG. 12A, for 10-cm long samples, only 33% of digital samples have 0.5 or greater pLCBV. As shown in FIG. 12B, for 20-cm long samples, only 12% of digital samples have 0.5 or greater pLCBV. As shown in FIG. 12C, for 25-cm long samples, none of the digital samples have 0.5 or greater pLCBV.

The results of this process may be employed for users designing sampling routines. They enter their BP, BS, and SCS into the logistic regression algorithm. That algorithm produces output of if burrows represent the connection of burrows across the 1 m model, for a particular SCS. If that burrow connectivity is represented, then any sample length with that SCS should represent the rock's burrow permeability appropriately. For the many SCS samples, where the output does not show 100% probability of burrows connecting across the 1 m sample, the user can take the additional step derived from the relationships outlined in FIGS. 11A-11C and FIGS. 12A-12C. To apply this, users may employ look-up Tables 5A-5D, and look-up Tables 6A-6D, choosing core plug or whole core SCS. They then must choose a sample length. This produces a probability of a sample, of that specified width and length, falling within the LCBV and correctly representing the permeability.

TABLE 4A
Length of LCBV for each combination of BS, BP, SCS

| BS | BP | SCS | Length | BS | BP | SCS | Length |
|---|---|---|---|---|---|---|---|
| 9 | 25 | 5 | 16 | 2.6 | 50 | 15 | 100 |
| 9 | 25 | 5 | 18 | 2.6 | 75 | 15 | 100 |
| 9 | 50 | 5 | 19 | 2.6 | 25 | 15 | 100 |
| 9 | 75 | 5 | 23 | 2.6 | 50 | 15 | 100 |
| 9 | 50 | 5 | 24 | 2.6 | 75 | 15 | 100 |
| 9 | 50 | 5 | 25 | 2.6 | 25 | 15 | 100 |
| 9 | 25 | 5 | 34 | 2.6 | 50 | 15 | 100 |
| 9 | 75 | 5 | 35 | 2.6 | 75 | 15 | 100 |
| 9 | 50 | 5 | 37 | 9 | 25 | 20 | 19 |
| 9 | 75 | 5 | 100 | 9 | 25 | 20 | 31 |
| 9 | 25 | 5 | 35 | 9 | 25 | 20 | 35 |
| 9 | 50 | 5 | 61 | 9 | 25 | 20 | 40 |
| 9 | 25 | 5 | 62 | 9 | 25 | 20 | 60 |
| 9 | 75 | 5 | 71 | 9 | 25 | 20 | 66 |
| 9 | 75 | 5 | 100 | 9 | 50 | 20 | 70 |
| 5 | 50 | 5 | 19 | 9 | 50 | 20 | 88 |
| 5.1 | 25 | 5 | 25 | 9 | 75 | 20 | 100 |
| 5.1 | 50 | 5 | 33 | 9 | 75 | 20 | 100 |
| 5.1 | 25 | 5 | 38 | 9 | 50 | 20 | 100 |
| 5.1 | 50 | 5 | 39 | 9 | 75 | 20 | 100 |
| 5.1 | 75 | 5 | 44 | 9 | 50 | 20 | 100 |
| 5.1 | 75 | 5 | 49 | 9 | 75 | 20 | 100 |
| 5.1 | 25 | 5 | 51 | 9 | 50 | 20 | 100 |
| 5.1 | 75 | 5 | 58 | 9 | 75 | 20 | 100 |
| 4 | 25 | 5 | 15 | 9 | 50 | 20 | 100 |
| 4 | 25 | 5 | 18 | 9 | 75 | 20 | 100 |
| 4 | 25 | 5 | 24 | 5.1 | 25 | 20 | 40 |
| 4 | 50 | 5 | 25 | 5.1 | 25 | 20 | 60 |
| 4 | 25 | 5 | 28 | 5.1 | 25 | 20 | 66 |
| 4 | 50 | 5 | 33 | 5.1 | 25 | 20 | 71 |
| 4 | 25 | 5 | 35 | 5.1 | 25 | 20 | 80 |
| 4 | 50 | 5 | 36 | 5.1 | 50 | 20 | 100 |
| 4 | 50 | 5 | 49 | 5.1 | 75 | 20 | 100 |
| 4 | 50 | 5 | 58 | 5.1 | 50 | 20 | 100 |
| 4 | 75 | 5 | 90 | 5.1 | 75 | 20 | 100 |
| 4 | 75 | 5 | 92 | 5.1 | 50 | 20 | 100 |
| 4 | 75 | 5 | 100 | 5.1 | 75 | 20 | 100 |
| 3.4 | 50 | 5 | 24 | 5.1 | 50 | 20 | 100 |
| 3.4 | 25 | 5 | 28 | 5.1 | 75 | 20 | 100 |
| 3.4 | 25 | 5 | 32 | 5.1 | 50 | 20 | 100 |
| 3.4 | 25 | 5 | 32 | 5.1 | 75 | 20 | 100 |
| 3.4 | 50 | 5 | 36 | 5.1 | 25 | 20 | 100 |
| 3.4 | 50 | 5 | 46 | 5.1 | 50 | 20 | 100 |
| 3.4 | 50 | 5 | 56 | 5.1 | 75 | 20 | 100 |
| 3.4 | 25 | 5 | 69 | 4 | 25 | 20 | 61 |
| 3.4 | 75 | 5 | 100 | 4 | 25 | 20 | 89 |
| 3.4 | 75 | 5 | 100 | 4 | 25 | 20 | 100 |
| 3.4 | 75 | 5 | 100 | 4 | 50 | 20 | 100 |
| 3.4 | 75 | 5 | 100 | 4 | 75 | 20 | 100 |
| 2.7 | 25 | 5 | 13 | 4 | 25 | 20 | 100 |
| 2.7 | 25 | 5 | 27 | 4 | 50 | 20 | 100 |
| 2.7 | 50 | 5 | 45 | 4 | 75 | 20 | 100 |
| 2.7 | 25 | 5 | 66 | 4 | 25 | 20 | 100 |
| 2.7 | 50 | 5 | 100 | 4 | 50 | 20 | 100 |
| 2.7 | 50 | 5 | 100 | 4 | 75 | 20 | 100 |
| 2.7 | 25 | 5 | 71 | 4 | 50 | 20 | 100 |
| 2.7 | 25 | 5 | 44 | 4 | 75 | 20 | 100 |
| 2.7 | 50 | 5 | 100 | 4 | 50 | 20 | 100 |
| 2.7 | 50 | 5 | 100 | 4 | 75 | 20 | 100 |
| 2.7 | 75 | 5 | 65 | 4 | 25 | 20 | 100 |
| 2.7 | 75 | 5 | 92 | 4 | 50 | 20 | 100 |
| 2.7 | 75 | 5 | 100 | 4 | 75 | 20 | 100 |
| 2.7 | 75 | 5 | 100 | 3.4 | 25 | 20 | 100 |
| 2.7 | 75 | 5 | 100 | 3.4 | 50 | 20 | 100 |
| 2.6 | 25 | 5 | 23 | 3.4 | 75 | 20 | 100 |
| 2.6 | 25 | 5 | 24 | 3.4 | 25 | 20 | 100 |
| 2.6 | 25 | 5 | 28 | 3.4 | 50 | 20 | 100 |
| 2.6 | 50 | 5 | 35 | 3.4 | 75 | 20 | 100 |
| 2.6 | 25 | 5 | 40 | 3.4 | 25 | 20 | 100 |
| 2.6 | 50 | 5 | 52 | 3.4 | 50 | 20 | 100 |
| 2.6 | 50 | 5 | 66 | 3.4 | 75 | 20 | 100 |
| 2.6 | 50 | 5 | 68 | 3.4 | 25 | 20 | 100 |
| 2.6 | 75 | 5 | 75 | 3.4 | 50 | 20 | 100 |
| 2.6 | 75 | 5 | 80 | 3.4 | 75 | 20 | 100 |
| 2.6 | 75 | 5 | 100 | 3.4 | 25 | 20 | 100 |
| 2.6 | 75 | 5 | 100 | 3.4 | 50 | 20 | 100 |
| 5.1 | 25 | 5 | 27 | 3.4 | 75 | 20 | 100 |
| 5.1 | 25 | 5 | 33 | 2.7 | 25 | 20 | 100 |
| 5.1 | 50 | 5 | 36 | 2.7 | 50 | 20 | 100 |
| 5.1 | 75 | 5 | 72 | 2.7 | 75 | 20 | 100 |
| 5.1 | 75 | 5 | 77 | 2.7 | 25 | 20 | 100 |
| 5.1 | 50 | 5 | 92 | 2.7 | 50 | 20 | 100 |
| 4 | 75 | 5 | 52 | 2.7 | 75 | 20 | 100 |
| 4 | 75 | 5 | 60 | 2.7 | 25 | 20 | 100 |
| 3.4 | 25 | 5 | 72 | 2.7 | 50 | 20 | 100 |
| 3.4 | 50 | 5 | 100 | 2.7 | 75 | 20 | 100 |
| 3.4 | 75 | 5 | 100 | 2.7 | 25 | 20 | 100 |
| 2.6 | 25 | 5 | 57 | 2.7 | 50 | 20 | 100 |
| 2.6 | 50 | 5 | 79 | 2.7 | 75 | 20 | 100 |
| 2.6 | 75 | 5 | 100 | 2.7 | 25 | 20 | 100 |
| 9 | 25 | 10 | 16 | 2.7 | 50 | 20 | 100 |
| 9 | 25 | 10 | 19 | 2.7 | 75 | 20 | 100 |
| 9 | 50 | 10 | 25 | 2.6 | 25 | 20 | 100 |
| 9 | 25 | 10 | 34 | 2.6 | 50 | 20 | 100 |
| 9 | 25 | 10 | 36 | 2.6 | 75 | 20 | 100 |
| 9 | 25 | 10 | 54 | 2.6 | 25 | 20 | 100 |
| 9 | 50 | 10 | 72 | 2.6 | 50 | 20 | 100 |
| 9 | 50 | 10 | 73 | 2.6 | 75 | 20 | 100 |
| 9 | 50 | 10 | 85 | 2.6 | 25 | 20 | 100 |
| 9 | 50 | 10 | 90 | 2.6 | 50 | 20 | 100 |
| 9 | 75 | 10 | 100 | 2.6 | 75 | 20 | 100 |
| 9 | 75 | 10 | 100 | 2.6 | 25 | 20 | 100 |
| 9 | 75 | 10 | 100 | 2.6 | 50 | 20 | 100 |
| 9 | 75 | 10 | 100 | 2.6 | 75 | 20 | 100 |
| 9 | 75 | 10 | 100 | 2.6 | 25 | 20 | 100 |
| 5.1 | 25 | 10 | 24 | 2.6 | 50 | 20 | 100 |
| 5.1 | 25 | 10 | 26 | 2.6 | 75 | 20 | 100 |
| 5.1 | 50 | 10 | 32 | 9 | 50 | 25 | 4 |
| 5.1 | 50 | 10 | 34 | 9 | 25 | 25 | 40 |
| 5.1 | 50 | 10 | 42 | 9 | 25 | 25 | 51 |
| 5.1 | 25 | 10 | 43 | 9 | 25 | 25 | 67 |
| 5.1 | 25 | 10 | 48 | 9 | 25 | 25 | 72 |
| 5.1 | 50 | 10 | 53 | 9 | 75 | 25 | 100 |
| 5.1 | 25 | 10 | 64 | 9 | 50 | 25 | 100 |
| 5.1 | 50 | 10 | 84 | 9 | 75 | 25 | 100 |
| 5.1 | 75 | 10 | 84 | 9 | 50 | 25 | 100 |
| 5.1 | 75 | 10 | 100 | 9 | 75 | 25 | 100 |
| 5.1 | 75 | 10 | 100 | 9 | 50 | 25 | 100 |
| 5.1 | 75 | 10 | 100 | 9 | 75 | 25 | 100 |
| 5.1 | 75 | 10 | 100 | 5.1 | 25 | 25 | 76 |
| 4 | 25 | 10 | 37 | 5.1 | 25 | 25 | 83 |
| 4 | 25 | 10 | 42 | 5.1 | 25 | 25 | 100 |
| 4 | 25 | 10 | 47 | 5.1 | 50 | 25 | 100 |
| 4 | 25 | 10 | 49 | 5.1 | 75 | 25 | 100 |
| 4 | 50 | 10 | 60 | 5.1 | 25 | 25 | 100 |
| 4 | 50 | 10 | 60 | 5.1 | 50 | 25 | 100 |
| 4 | 50 | 10 | 64 | 5.1 | 75 | 25 | 100 |
| 4 | 50 | 10 | 74 | 5.1 | 50 | 25 | 100 |
| 4 | 25 | 10 | 100 | 5.1 | 75 | 25 | 100 |
| 4 | 75 | 10 | 100 | 5.1 | 50 | 25 | 100 |
| 4 | 75 | 10 | 100 | 5.1 | 75 | 25 | 100 |
| 4 | 50 | 10 | 100 | 4 | 25 | 25 | 51 |
| 4 | 75 | 10 | 100 | 4 | 25 | 25 | 100 |
| 4 | 75 | 10 | 100 | 4 | 50 | 25 | 100 |
| 4 | 75 | 10 | 100 | 4 | 75 | 25 | 100 |
| 3.4 | 25 | 10 | 49 | 4 | 25 | 25 | 100 |
| 3.4 | 25 | 10 | 49 | 4 | 50 | 25 | 100 |
| 3.4 | 25 | 10 | 64 | 4 | 75 | 25 | 100 |
| 3.4 | 25 | 10 | 96 | 4 | 50 | 25 | 100 |
| 3.4 | 50 | 10 | 100 | 4 | 75 | 25 | 100 |
| 3.4 | 75 | 10 | 100 | 4 | 25 | 25 | 100 |
| 3.4 | 50 | 10 | 100 | 4 | 50 | 25 | 100 |
| 3.4 | 75 | 10 | 100 | 4 | 75 | 25 | 100 |
| 3.4 | 50 | 10 | 100 | 3.4 | 25 | 25 | 100 |
| 3.4 | 75 | 10 | 100 | 3.4 | 50 | 25 | 100 |
| 3.4 | 50 | 10 | 100 | 3.4 | 75 | 25 | 100 |
| 3.4 | 75 | 10 | 100 | 3.4 | 25 | 25 | 100 |
| 3.4 | 25 | 10 | 100 | 3.4 | 50 | 25 | 100 |
| 3.4 | 50 | 10 | 100 | 3.4 | 75 | 25 | 100 |
| 3.4 | 75 | 10 | 100 | 3.4 | 25 | 25 | 100 |
| 2.7 | 25 | 10 | 80 | 3.4 | 50 | 25 | 100 |
| 2.7 | 25 | 10 | 50 | 3.4 | 75 | 25 | 100 |
| 2.7 | 50 | 10 | 64 | 3.4 | 25 | 25 | 100 |
| 2.7 | 25 | 10 | 98 | 3.4 | 50 | 25 | 100 |
| 2.7 | 25 | 10 | 96 | 3.4 | 75 | 25 | 100 |
| 2.7 | 50 | 10 | 100 | 2.7 | 25 | 25 | 100 |
| 2.7 | 50 | 10 | 76 | 2.7 | 50 | 25 | 100 |
| 2.7 | 25 | 10 | 100 | 2.7 | 75 | 25 | 100 |
| 2.7 | 50 | 10 | 100 | 2.7 | 25 | 25 | 100 |
| 2.7 | 75 | 10 | 100 | 2.7 | 50 | 25 | 100 |
| 2.7 | 75 | 10 | 100 | 2.7 | 75 | 25 | 100 |
| 2.7 | 75 | 10 | 100 | 2.7 | 25 | 25 | 100 |
| 2.7 | 50 | 10 | 100 | 2.7 | 50 | 25 | 100 |
| 2.7 | 75 | 10 | 100 | 2.7 | 75 | 25 | 100 |
| 2.7 | 75 | 10 | 100 | 2.7 | 25 | 25 | 100 |
| 2.6 | 25 | 10 | 67 | 2.7 | 50 | 25 | 100 |
| 2.6 | 25 | 10 | 80 | 2.7 | 75 | 25 | 100 |
| 2.6 | 25 | 10 | 91 | 2.6 | 25 | 25 | 100 |
| 2.6 | 25 | 10 | 100 | 2.6 | 50 | 25 | 100 |
| 2.6 | 50 | 10 | 100 | 2.6 | 75 | 25 | 100 |
| 2.6 | 75 | 10 | 100 | 2.6 | 25 | 25 | 100 |
| 2.6 | 50 | 10 | 100 | 2.6 | 50 | 25 | 100 |
| 2.6 | 75 | 10 | 100 | 2.6 | 75 | 25 | 100 |
| 2.6 | 25 | 10 | 100 | 2.6 | 25 | 25 | 100 |
| 2.6 | 50 | 10 | 100 | 2.6 | 50 | 25 | 100 |
| 2.6 | 75 | 10 | 100 | 2.6 | 75 | 25 | 100 |
| 2.6 | 50 | 10 | 100 | 2.6 | 25 | 25 | 100 |
| 2.6 | 75 | 10 | 100 | 2.6 | 50 | 25 | 100 |
| 2.6 | 50 | 10 | 100 | 2.6 | 75 | 25 | 100 |
| 2.6 | 75 | 10 | 100 | 9 | 25 | 30 | 15 |
| 9 | 25 | 15 | 22 | 9 | 50 | 30 | 30 |
| 9 | 25 | 15 | 41 | 9 | 25 | 30 | 66 |
| 9 | 25 | 15 | 49 | 9 | 25 | 30 | 100 |
| 9 | 25 | 15 | 65 | 9 | 50 | 30 | 100 |
| 9 | 50 | 15 | 74 | 9 | 75 | 30 | 100 |
| 9 | 50 | 15 | 82 | 9 | 50 | 30 | 100 |
| 9 | 50 | 15 | 100 | 9 | 75 | 30 | 100 |
| 9 | 75 | 15 | 100 | 9 | 25 | 30 | 100 |
| 9 | 75 | 15 | 100 | 9 | 50 | 30 | 100 |
| 9 | 75 | 15 | 100 | 9 | 75 | 30 | 100 |
| 9 | 50 | 15 | 100 | 9 | 75 | 30 | 100 |
| 9 | 75 | 15 | 100 | 5.1 | 25 | 30 | 71 |
| 5.1 | 25 | 15 | 30 | 5.1 | 25 | 30 | 100 |
| 5.1 | 25 | 15 | 39 | 5.1 | 50 | 30 | 100 |
| 5.1 | 25 | 15 | 59 | 5.1 | 75 | 30 | 100 |
| 5.1 | 50 | 15 | 78 | 5.1 | 25 | 30 | 100 |
| 5.1 | 75 | 15 | 79 | 5.1 | 50 | 30 | 100 |
| 5.1 | 50 | 15 | 95 | 5.1 | 75 | 30 | 100 |
| 5.1 | 50 | 15 | 100 | 5.1 | 50 | 30 | 100 |
| 5.1 | 75 | 15 | 100 | 5.1 | 75 | 30 | 100 |
| 5.1 | 50 | 15 | 100 | 5.1 | 25 | 30 | 100 |
| 5.1 | 75 | 15 | 100 | 5.1 | 50 | 30 | 100 |
| 5.1 | 25 | 15 | 100 | 5.1 | 75 | 30 | 100 |
| 5.1 | 75 | 15 | 100 | 4 | 25 | 30 | 100 |
| 4 | 25 | 15 | 40 | 4 | 50 | 30 | 100 |
| 4 | 75 | 15 | 48 | 4 | 75 | 30 | 100 |
| 4 | 25 | 15 | 51 | 4 | 25 | 30 | 100 |
| 4 | 25 | 15 | 82 | 4 | 50 | 30 | 100 |
| 4 | 25 | 15 | 84 | 4 | 75 | 30 | 100 |
| 4 | 50 | 15 | 100 | 4 | 25 | 30 | 100 |
| 4 | 50 | 15 | 100 | 4 | 50 | 30 | 100 |
| 4 | 75 | 15 | 100 | 4 | 75 | 30 | 100 |
| 4 | 50 | 15 | 100 | 4 | 25 | 30 | 100 |
| 4 | 75 | 15 | 100 | 4 | 50 | 30 | 100 |
| 4 | 50 | 15 | 100 | 4 | 75 | 30 | 100 |
| 4 | 75 | 15 | 100 | 3.4 | 25 | 30 | 100 |
| 3.4 | 25 | 15 | 54 | 3.4 | 50 | 30 | 100 |
| 3.4 | 25 | 15 | 64 | 3.4 | 75 | 30 | 100 |
| 3.4 | 25 | 15 | 99 | 3.4 | 25 | 30 | 100 |
| 3.4 | 50 | 15 | 100 | 3.4 | 50 | 30 | 100 |
| 3.4 | 75 | 15 | 100 | 3.4 | 75 | 30 | 100 |
| 3.4 | 25 | 15 | 100 | 3.4 | 25 | 30 | 100 |
| 3.4 | 50 | 15 | 100 | 3.4 | 50 | 30 | 100 |
| 3.4 | 75 | 15 | 100 | 3.4 | 75 | 30 | 100 |
| 3.4 | 50 | 15 | 100 | 3.4 | 25 | 30 | 100 |
| 3.4 | 75 | 15 | 100 | 3.4 | 50 | 30 | 100 |
| 3.4 | 50 | 15 | 100 | 3.4 | 75 | 30 | 100 |
| 3.4 | 75 | 15 | 100 | 2.7 | 25 | 30 | 100 |
| 3.4 | 25 | 15 | 100 | 2.7 | 50 | 30 | 100 |
| 3.4 | 50 | 15 | 100 | 2.7 | 75 | 30 | 100 |
| 3.4 | 75 | 15 | 100 | 2.7 | 25 | 30 | 100 |
| 2.7 | 25 | 15 | 100 | 2.7 | 50 | 30 | 100 |
| 2.7 | 50 | 15 | 100 | 2.7 | 75 | 30 | 100 |
| 2.7 | 75 | 15 | 100 | 2.7 | 25 | 30 | 100 |
| 2.7 | 25 | 15 | 100 | 2.7 | 50 | 30 | 100 |
| 2.7 | 50 | 15 | 100 | 2.7 | 75 | 30 | 100 |
| 2.7 | 75 | 15 | 100 | 2.7 | 25 | 30 | 100 |
| 2.7 | 25 | 15 | 100 | 2.7 | 50 | 30 | 100 |
| 2.7 | 50 | 15 | 100 | 2.7 | 75 | 30 | 100 |
| 2.7 | 75 | 15 | 100 | 2.6 | 25 | 30 | 100 |
| 2.7 | 25 | 15 | 100 | 2.6 | 50 | 30 | 100 |
| 2.7 | 50 | 15 | 100 | 2.6 | 75 | 30 | 100 |
| 2.7 | 75 | 15 | 100 | 2.6 | 25 | 30 | 100 |
| 2.7 | 25 | 15 | 100 | 2.6 | 50 | 30 | 100 |
| 2.7 | 50 | 15 | 100 | 2.6 | 75 | 30 | 100 |
| 2.7 | 75 | 15 | 100 | 2.6 | 25 | 30 | 100 |
| 2.6 | 25 | 15 | 100 | 2.6 | 50 | 30 | 100 |
| 2.6 | 50 | 15 | 100 | 2.6 | 75 | 30 | 100 |
| 2.6 | 75 | 15 | 100 | 2.6 | 25 | 30 | 100 |
| 2.6 | 25 | 15 | 100 | 2.6 | 50 | 30 | 100 |
| 2.6 | 50 | 15 | 100 | 2.6 | 75 | 30 | 100 |
| 2.6 | 75 | 15 | 100 | | | | |
| 2.6 | 25 | 15 | 100 | | | | |

TABLE 4B
Burrow connectivity for each combination of BS, BP, SCS

| | | | Burrow | | | | Burrow |
|---|---|---|---|---|---|---|---|
| BS | BP | SCS | connectivity | BS | BP | SCS | connectivity |
| 2.6 | 25 | 5 | 0 | 4 | 25 | 5 | 0 |
| 2.6 | 25 | 5 | 0 | 4 | 25 | 5 | 0 |
| 2.6 | 25 | 5 | 0 | 4 | 25 | 5 | 0 |
| 2.6 | 25 | 5 | 0 | 4 | 25 | 5 | 0 |
| 2.6 | 25 | 5 | 0 | 4 | 25 | 5 | 0 |
| 2.6 | 25 | 10 | 0 | 4 | 25 | 10 | 0 |
| 2.6 | 25 | 10 | 0 | 4 | 25 | 10 | 0 |
| 2.6 | 25 | 10 | 0 | 4 | 25 | 10 | 0 |
| 2.6 | 25 | 10 | 1 | 4 | 25 | 10 | 1 |
| 2.6 | 25 | 10 | 1 | 4 | 25 | 10 | 1 |
| 2.6 | 25 | 15 | 0 | 4 | 25 | 15 | 0 |
| 2.6 | 25 | 15 | 1 | 4 | 25 | 15 | 0 |
| 2.6 | 25 | 15 | 1 | 4 | 25 | 15 | 0 |
| 2.6 | 25 | 15 | 1 | 4 | 25 | 15 | 0 |
| 2.6 | 25 | 15 | 1 | 4 | 25 | 15 | 1 |
| 2.6 | 25 | 20 | 1 | 4 | 25 | 20 | 0 |
| 2.6 | 25 | 20 | 1 | 4 | 25 | 20 | 1 |
| 2.6 | 25 | 20 | 1 | 4 | 25 | 20 | 1 |
| 2.6 | 25 | 20 | 1 | 4 | 25 | 20 | 1 |
| 2.6 | 25 | 20 | 1 | 4 | 25 | 20 | 1 |
| 2.6 | 25 | 25 | 1 | 4 | 25 | 25 | 0 |
| 2.6 | 25 | 25 | 1 | 4 | 25 | 25 | 1 |
| 2.6 | 25 | 25 | 1 | 4 | 25 | 25 | 1 |
| 2.6 | 25 | 25 | 1 | 4 | 25 | 25 | 1 |
| 2.6 | 25 | 25 | 1 | 4 | 25 | 25 | 1 |
| 2.6 | 25 | 30 | 1 | 4 | 25 | 30 | 1 |
| 2.6 | 25 | 30 | 1 | 4 | 25 | 30 | 1 |
| 2.6 | 25 | 30 | 1 | 4 | 25 | 30 | 1 |
| 2.6 | 25 | 30 | 1 | 4 | 25 | 30 | 1 |
| 2.6 | 25 | 30 | 1 | 4 | 25 | 30 | 1 |
| 2.6 | 50 | 5 | 0 | 4 | 50 | 5 | 0 |
| 2.6 | 50 | 5 | 0 | 4 | 50 | 5 | 0 |
| 2.6 | 50 | 5 | 0 | 4 | 50 | 5 | 0 |
| 2.6 | 50 | 5 | 0 | 4 | 50 | 5 | 0 |
| 2.6 | 50 | 5 | 0 | 4 | 50 | 5 | 0 |
| 2.6 | 50 | 10 | 1 | 4 | 50 | 10 | 0 |
| 2.6 | 50 | 10 | 1 | 4 | 50 | 10 | 0 |
| 2.6 | 50 | 10 | 1 | 4 | 50 | 10 | 0 |
| 2.6 | 50 | 10 | 1 | 4 | 50 | 10 | 0 |
| 2.6 | 50 | 10 | 1 | 4 | 50 | 10 | 1 |
| 2.6 | 50 | 15 | 1 | 4 | 50 | 15 | 1 |
| 2.6 | 50 | 15 | 1 | 4 | 50 | 15 | 1 |
| 2.6 | 50 | 15 | 1 | 4 | 50 | 15 | 1 |
| 2.6 | 50 | 15 | 1 | 4 | 50 | 15 | 1 |
| 2.6 | 50 | 15 | 1 | 4 | 50 | 15 | 1 |
| 2.6 | 50 | 20 | 1 | 4 | 50 | 20 | 1 |
| 2.6 | 50 | 20 | 1 | 4 | 50 | 20 | 1 |
| 2.6 | 50 | 20 | 1 | 4 | 50 | 20 | 1 |
| 2.6 | 50 | 20 | 1 | 4 | 50 | 20 | 1 |
| 2.6 | 50 | 20 | 1 | 4 | 50 | 20 | 1 |
| 2.6 | 50 | 25 | 1 | 4 | 50 | 25 | 1 |
| 2.6 | 50 | 25 | 1 | 4 | 50 | 25 | 1 |
| 2.6 | 50 | 25 | 1 | 4 | 50 | 25 | 1 |
| 2.6 | 50 | 25 | 1 | 4 | 50 | 25 | 1 |
| 2.6 | 50 | 25 | 1 | 4 | 50 | 25 | 1 |
| 2.6 | 50 | 30 | 1 | 4 | 50 | 30 | 1 |
| 2.6 | 50 | 30 | 1 | 4 | 50 | 30 | 1 |
| 2.6 | 50 | 30 | 1 | 4 | 50 | 30 | 1 |
| 2.6 | 50 | 30 | 1 | 4 | 50 | 30 | 1 |
| 2.6 | 50 | 30 | 1 | 4 | 50 | 30 | 1 |
| 2.6 | 75 | 5 | 1 | 4 | 75 | 5 | 0 |
| 2.6 | 75 | 5 | 1 | 4 | 75 | 5 | 0 |
| 2.6 | 75 | 5 | 1 | 4 | 75 | 5 | 0 |
| 2.6 | 75 | 5 | 1 | 4 | 75 | 5 | 0 |
| 2.6 | 75 | 5 | 1 | 4 | 75 | 5 | 1 |
| 2.6 | 75 | 10 | 1 | 4 | 75 | 10 | 1 |
| 2.6 | 75 | 10 | 1 | 4 | 75 | 10 | 1 |
| 2.6 | 75 | 10 | 1 | 4 | 75 | 10 | 1 |
| 2.6 | 75 | 10 | 1 | 4 | 75 | 10 | 1 |
| 2.6 | 75 | 10 | 1 | 4 | 75 | 10 | 1 |
| 2.6 | 75 | 15 | 1 | 4 | 75 | 15 | 1 |
| 2.6 | 75 | 15 | 1 | 4 | 75 | 15 | 1 |
| 2.6 | 75 | 15 | 1 | 4 | 75 | 15 | 1 |
| 2.6 | 75 | 15 | 1 | 4 | 75 | 15 | 1 |
| 2.6 | 75 | 15 | 1 | 4 | 75 | 15 | 1 |
| 2.6 | 75 | 20 | 1 | 4 | 75 | 20 | 1 |
| 2.6 | 75 | 20 | 1 | 4 | 75 | 20 | 1 |
| 2.6 | 75 | 20 | 1 | 4 | 75 | 20 | 1 |
| 2.6 | 75 | 20 | 1 | 4 | 75 | 20 | 1 |
| 2.6 | 75 | 20 | 1 | 4 | 75 | 20 | 1 |
| 2.6 | 75 | 25 | 1 | 4 | 75 | 25 | 1 |
| 2.6 | 75 | 25 | 1 | 4 | 75 | 25 | 1 |
| 2.6 | 75 | 25 | 1 | 4 | 75 | 25 | 1 |
| 2.6 | 75 | 25 | 1 | 4 | 75 | 25 | 1 |
| 2.6 | 75 | 25 | 1 | 4 | 75 | 25 | 1 |
| 2.6 | 75 | 30 | 1 | 4 | 75 | 30 | 1 |
| 2.6 | 75 | 30 | 1 | 4 | 75 | 30 | 1 |
| 2.6 | 75 | 30 | 1 | 4 | 75 | 30 | 1 |
| 2.6 | 75 | 30 | 1 | 4 | 75 | 30 | 1 |
| 2.6 | 75 | 30 | 1 | 4 | 75 | 30 | 1 |
| 2.7 | 25 | 5 | 0 | 5.1 | 25 | 5 | 0 |
| 2.7 | 25 | 5 | 0 | 5.1 | 25 | 5 | 0 |
| 2.7 | 25 | 5 | 0 | 5.1 | 25 | 5 | 0 |
| 2.7 | 25 | 5 | 0 | 5.1 | 25 | 5 | 0 |
| 2.7 | 25 | 5 | 0 | 5.1 | 25 | 5 | 0 |
| 2.7 | 25 | 10 | 0 | 5.1 | 25 | 10 | 0 |
| 2.7 | 25 | 10 | 0 | 5.1 | 25 | 10 | 0 |
| 2.7 | 25 | 10 | 0 | 5.1 | 25 | 10 | 0 |
| 2.7 | 25 | 10 | 0 | 5.1 | 25 | 10 | 0 |
| 2.7 | 25 | 10 | 0 | 5.1 | 25 | 10 | 0 |
| 2.7 | 25 | 15 | 0 | 5.1 | 25 | 15 | 0 |
| 2.7 | 25 | 15 | 1 | 5.1 | 25 | 15 | 0 |
| 2.7 | 25 | 15 | 1 | 5.1 | 25 | 15 | 0 |
| 2.7 | 25 | 15 | 1 | 5.1 | 25 | 15 | 0 |
| 2.7 | 25 | 15 | 1 | 5.1 | 25 | 15 | 1 |
| 2.7 | 25 | 20 | 1 | 5.1 | 25 | 20 | 0 |
| 2.7 | 25 | 20 | 1 | 5.1 | 25 | 20 | 0 |
| 2.7 | 25 | 20 | 1 | 5.1 | 25 | 20 | 0 |
| 2.7 | 25 | 20 | 1 | 5.1 | 25 | 20 | 0 |
| 2.7 | 25 | 20 | 1 | 5.1 | 25 | 20 | 1 |
| 2.7 | 25 | 25 | 1 | 5.1 | 25 | 25 | 0 |
| 2.7 | 25 | 25 | 1 | 5.1 | 25 | 25 | 0 |
| 2.7 | 25 | 25 | 1 | 5.1 | 25 | 25 | 1 |
| 2.7 | 25 | 25 | 1 | 5.1 | 25 | 25 | 1 |
| 2.7 | 25 | 25 | 1 | 5.1 | 25 | 25 | 1 |
| 2.7 | 25 | 30 | 1 | 5.1 | 25 | 30 | 0 |
| 2.7 | 25 | 30 | 1 | 5.1 | 25 | 30 | 1 |
| 2.7 | 25 | 30 | 1 | 5.1 | 25 | 30 | 1 |
| 2.7 | 25 | 30 | 1 | 5.1 | 25 | 30 | 1 |
| 2.7 | 25 | 30 | 1 | 5.1 | 25 | 30 | 1 |
| 2.7 | 50 | 5 | 0 | 5.1 | 50 | 5 | 0 |
| 2.7 | 50 | 5 | 0 | 5.1 | 50 | 5 | 0 |
| 2.7 | 50 | 5 | 0 | 5.1 | 50 | 5 | 0 |
| 2.7 | 50 | 5 | 0 | 5.1 | 50 | 5 | 0 |
| 2.7 | 50 | 5 | 0 | 5.1 | 50 | 5 | 0 |
| 2.7 | 50 | 10 | 0 | 5.1 | 50 | 10 | 0 |
| 2.7 | 50 | 10 | 0 | 5.1 | 50 | 10 | 0 |
| 2.7 | 50 | 10 | 0 | 5.1 | 50 | 10 | 0 |
| 2.7 | 50 | 10 | 1 | 5.1 | 50 | 10 | 0 |
| 2.7 | 50 | 10 | 1 | 5.1 | 50 | 10 | 0 |
| 2.7 | 50 | 15 | 1 | 5.1 | 50 | 15 | 0 |
| 2.7 | 50 | 15 | 1 | 5.1 | 50 | 15 | 0 |
| 2.7 | 50 | 15 | 1 | 5.1 | 50 | 15 | 0 |
| 2.7 | 50 | 15 | 1 | 5.1 | 50 | 15 | 1 |
| 2.7 | 50 | 15 | 1 | 5.1 | 50 | 15 | 1 |
| 2.7 | 50 | 20 | 1 | 5.1 | 50 | 20 | 1 |
| 2.7 | 50 | 20 | 1 | 5.1 | 50 | 20 | 1 |
| 2.7 | 50 | 20 | 1 | 5.1 | 50 | 20 | 1 |
| 2.7 | 50 | 20 | 1 | 5.1 | 50 | 20 | 1 |
| 2.7 | 50 | 20 | 1 | 5.1 | 50 | 20 | 1 |
| 2.7 | 50 | 25 | 1 | 5.1 | 50 | 25 | 1 |
| 2.7 | 50 | 25 | 1 | 5.1 | 50 | 25 | 1 |
| 2.7 | 50 | 25 | 1 | 5.1 | 50 | 25 | 1 |
| 2.7 | 50 | 25 | 1 | 5.1 | 50 | 25 | 1 |
| 2.7 | 50 | 25 | 1 | 5.1 | 50 | 25 | 1 |
| 2.7 | 50 | 30 | 1 | 5.1 | 50 | 30 | 1 |
| 2.7 | 50 | 30 | 1 | 5.1 | 50 | 30 | 1 |
| 2.7 | 50 | 30 | 1 | 5.1 | 50 | 30 | 1 |
| 2.7 | 50 | 30 | 1 | 5.1 | 50 | 30 | 1 |
| 2.7 | 50 | 30 | 1 | 5.1 | 50 | 30 | 1 |
| 2.7 | 75 | 5 | 0 | 5.1 | 75 | 5 | 0 |
| 2.7 | 75 | 5 | 0 | 5.1 | 75 | 5 | 0 |
| 2.7 | 75 | 5 | 1 | 5.1 | 75 | 5 | 0 |
| 2.7 | 75 | 5 | 1 | 5.1 | 75 | 5 | 0 |
| 2.7 | 75 | 5 | 1 | 5.1 | 75 | 5 | 1 |
| 2.7 | 75 | 10 | 1 | 5.1 | 75 | 10 | 0 |
| 2.7 | 75 | 10 | 1 | 5.1 | 75 | 10 | 0 |
| 2.7 | 75 | 10 | 1 | 5.1 | 75 | 10 | 1 |
| 2.7 | 75 | 10 | 1 | 5.1 | 75 | 10 | 1 |
| 2.7 | 75 | 10 | 1 | 5.1 | 75 | 10 | 1 |
| 2.7 | 75 | 15 | 1 | 5.1 | 75 | 15 | 1 |
| 2.7 | 75 | 15 | 1 | 5.1 | 75 | 15 | 1 |
| 2.7 | 75 | 15 | 1 | 5.1 | 75 | 15 | 1 |
| 2.7 | 75 | 15 | 1 | 5.1 | 75 | 15 | 1 |
| 2.7 | 75 | 15 | 1 | 5.1 | 75 | 15 | 1 |
| 2.7 | 75 | 20 | 1 | 5.1 | 75 | 20 | 1 |
| 2.7 | 75 | 20 | 1 | 5.1 | 75 | 20 | 1 |
| 2.7 | 75 | 20 | 1 | 5.1 | 75 | 20 | 1 |
| 2.7 | 75 | 20 | 1 | 5.1 | 75 | 20 | 1 |
| 2.7 | 75 | 20 | 1 | 5.1 | 75 | 20 | 1 |
| 2.7 | 75 | 25 | 1 | 5.1 | 75 | 25 | 1 |
| 2.7 | 75 | 25 | 1 | 5.1 | 75 | 25 | 1 |
| 2.7 | 75 | 25 | 1 | 5.1 | 75 | 25 | 1 |
| 2.7 | 75 | 25 | 1 | 5.1 | 75 | 25 | 1 |
| 2.7 | 75 | 25 | 1 | 5.1 | 75 | 25 | 1 |
| 2.7 | 75 | 30 | 1 | 5.1 | 75 | 30 | 1 |
| 2.7 | 75 | 30 | 1 | 5.1 | 75 | 30 | 1 |
| 2.7 | 75 | 30 | 1 | 5.1 | 75 | 30 | 1 |
| 2.7 | 75 | 30 | 1 | 5.1 | 75 | 30 | 1 |
| 2.7 | 75 | 30 | 1 | 5.1 | 75 | 30 | 1 |
| 3.4 | 25 | 5 | 0 | 9 | 25 | 5 | 0 |
| 3.4 | 25 | 5 | 0 | 9 | 25 | 5 | 0 |
| 3.4 | 25 | 5 | 0 | 9 | 25 | 5 | 0 |
| 3.4 | 25 | 5 | 0 | 9 | 25 | 5 | 0 |
| 3.4 | 25 | 5 | 0 | 9 | 25 | 5 | 0 |
| 3.4 | 25 | 10 | 0 | 9 | 25 | 10 | 0 |
| 3.4 | 25 | 10 | 0 | 9 | 25 | 10 | 0 |
| 3.4 | 25 | 10 | 0 | 9 | 25 | 10 | 0 |
| 3.4 | 25 | 10 | 0 | 9 | 25 | 10 | 0 |
| 3.4 | 25 | 10 | 0 | 9 | 25 | 10 | 0 |
| 3.4 | 25 | 15 | 0 | 9 | 25 | 15 | 0 |
| 3.4 | 25 | 15 | 0 | 9 | 25 | 15 | 0 |
| 3.4 | 25 | 15 | 0 | 9 | 25 | 15 | 0 |
| 3.4 | 25 | 15 | 1 | 9 | 25 | 15 | 0 |
| 3.4 | 25 | 15 | 1 | 9 | 25 | 15 | 0 |
| 3.4 | 25 | 20 | 0 | 9 | 25 | 20 | 0 |
| 3.4 | 25 | 20 | 1 | 9 | 25 | 20 | 0 |
| 3.4 | 25 | 20 | 1 | 9 | 25 | 20 | 0 |
| 3.4 | 25 | 20 | 1 | 9 | 25 | 20 | 0 |
| 3.4 | 25 | 20 | 1 | 9 | 25 | 20 | 0 |
| 3.4 | 25 | 25 | 1 | 9 | 25 | 25 | 0 |
| 3.4 | 25 | 25 | 1 | 9 | 25 | 25 | 0 |
| 3.4 | 25 | 25 | 1 | 9 | 25 | 25 | 0 |
| 3.4 | 25 | 25 | 1 | 9 | 25 | 25 | 0 |
| 3.4 | 25 | 25 | 1 | 9 | 25 | 25 | 0 |
| 3.4 | 25 | 30 | 1 | 9 | 25 | 30 | 0 |
| 3.4 | 25 | 30 | 1 | 9 | 25 | 30 | 0 |
| 3.4 | 25 | 30 | 1 | 9 | 25 | 30 | 0 |
| 3.4 | 25 | 30 | 1 | 9 | 25 | 30 | 1 |
| 3.4 | 25 | 30 | 1 | 9 | 25 | 30 | 1 |
| 3.4 | 50 | 5 | 0 | 9 | 50 | 5 | 0 |
| 3.4 | 50 | 5 | 0 | 9 | 50 | 5 | 0 |
| 3.4 | 50 | 5 | 0 | 9 | 50 | 5 | 0 |
| 3.4 | 50 | 5 | 0 | 9 | 50 | 5 | 0 |
| 3.4 | 50 | 5 | 0 | 9 | 50 | 5 | 0 |
| 3.4 | 50 | 10 | 1 | 9 | 50 | 10 | 0 |
| 3.4 | 50 | 10 | 1 | 9 | 50 | 10 | 0 |
| 3.4 | 50 | 10 | 1 | 9 | 50 | 10 | 0 |
| 3.4 | 50 | 10 | 1 | 9 | 50 | 10 | 0 |
| 3.4 | 50 | 10 | 1 | 9 | 50 | 10 | 0 |
| 3.4 | 50 | 15 | 1 | 9 | 50 | 15 | 0 |
| 3.4 | 50 | 15 | 1 | 9 | 50 | 15 | 0 |
| 3.4 | 50 | 15 | 1 | 9 | 50 | 15 | 0 |
| 3.4 | 50 | 15 | 1 | 9 | 50 | 15 | 0 |
| 3.4 | 50 | 15 | 1 | 9 | 50 | 15 | 1 |
| 3.4 | 50 | 20 | 1 | 9 | 50 | 20 | 0 |
| 3.4 | 50 | 20 | 1 | 9 | 50 | 20 | 0 |
| 3.4 | 50 | 20 | 1 | 9 | 50 | 20 | 0 |
| 3.4 | 50 | 20 | 1 | 9 | 50 | 20 | 1 |
| 3.4 | 50 | 20 | 1 | 9 | 50 | 20 | 1 |
| 3.4 | 50 | 25 | 1 | 9 | 50 | 25 | 0 |
| 3.4 | 50 | 25 | 1 | 9 | 50 | 25 | 1 |
| 3.4 | 50 | 25 | 1 | 9 | 50 | 25 | 1 |
| 3.4 | 50 | 25 | 1 | 9 | 50 | 25 | 1 |
| 3.4 | 50 | 25 | 1 | 9 | 50 | 25 | 1 |
| 3.4 | 50 | 30 | 1 | 9 | 50 | 30 | 1 |
| 3.4 | 50 | 30 | 1 | 9 | 50 | 30 | 1 |
| 3.4 | 50 | 30 | 1 | 9 | 50 | 30 | 1 |
| 3.4 | 50 | 30 | 1 | 9 | 50 | 30 | 1 |
| 3.4 | 50 | 30 | 1 | 9 | 50 | 30 | 1 |
| 3.4 | 75 | 5 | 0 | 9 | 75 | 5 | 0 |
| 3.4 | 75 | 5 | 0 | 9 | 75 | 5 | 0 |
| 3.4 | 75 | 5 | 1 | 9 | 75 | 5 | 0 |
| 3.4 | 75 | 5 | 1 | 9 | 75 | 5 | 1 |
| 3.4 | 75 | 5 | 1 | 9 | 75 | 5 | 1 |
| 3.4 | 75 | 10 | 1 | 9 | 75 | 10 | 0 |
| 3.4 | 75 | 10 | 1 | 9 | 75 | 10 | 1 |
| 3.4 | 75 | 10 | 1 | 9 | 75 | 10 | 1 |
| 3.4 | 75 | 10 | 1 | 9 | 75 | 10 | 1 |
| 3.4 | 75 | 10 | 1 | 9 | 75 | 10 | 1 |
| 3.4 | 75 | 15 | 1 | 9 | 75 | 15 | 1 |
| 3.4 | 75 | 15 | 1 | 9 | 75 | 15 | 1 |
| 3.4 | 75 | 15 | 1 | 9 | 75 | 15 | 1 |
| 3.4 | 75 | 15 | 1 | 9 | 75 | 15 | 1 |
| 3.4 | 75 | 15 | 1 | 9 | 75 | 15 | 1 |
| 3.4 | 75 | 20 | 1 | 9 | 75 | 20 | 1 |
| 3.4 | 75 | 20 | 1 | 9 | 75 | 20 | 1 |
| 3.4 | 75 | 20 | 1 | 9 | 75 | 20 | 1 |
| 3.4 | 75 | 20 | 1 | 9 | 75 | 20 | 1 |
| 3.4 | 75 | 20 | 1 | 9 | 75 | 20 | 1 |
| 3.4 | 75 | 25 | 1 | 9 | 75 | 25 | 1 |
| 3.4 | 75 | 25 | 1 | 9 | 75 | 25 | 1 |
| 3.4 | 75 | 25 | 1 | 9 | 75 | 25 | 1 |
| 3.4 | 75 | 25 | 1 | 9 | 75 | 25 | 1 |
| 3.4 | 75 | 25 | 1 | 9 | 75 | 25 | 1 |
| 3.4 | 75 | 30 | 1 | 9 | 75 | 30 | 1 |
| 3.4 | 75 | 30 | 1 | 9 | 75 | 30 | 1 |
| 3.4 | 75 | 30 | 1 | 9 | 75 | 30 | 1 |
| 3.4 | 75 | 30 | 1 | 9 | 75 | 30 | 1 |
| 3.4 | 75 | 30 | 1 | 9 | 75 | 30 | 1 |

TABLE 5A
Data pertaining to MPS models with cross section of about 25 cm²

| | | | Length | | | | Length |
|---|---|---|---|---|---|---|---|
| BS | BP | SCS | (L) cm | BS | BP | SCS | (L) cm |
| 2.7 | 25 | 25 | 13 | 5.1 | 25 | 25 | 38 |
| 4 | 25 | 25 | 15 | 5.1 | 50 | 25 | 39 |
| 9 | 25 | 25 | 16 | 2.6 | 25 | 25 | 40 |
| 9 | 25 | 25 | 18 | 5.1 | 75 | 25 | 44 |
| 4 | 25 | 25 | 18 | 2.7 | 25 | 25 | 44 |
| 9 | 50 | 25 | 19 | 2.7 | 50 | 25 | 45 |
| 5.1 | 50 | 25 | 19 | 3.4 | 50 | 25 | 46 |
| 9 | 75 | 25 | 23 | 5.1 | 75 | 25 | 49 |
| 2.6 | 25 | 25 | 23 | 4 | 50 | 25 | 49 |
| 9 | 50 | 25 | 24 | 5.1 | 25 | 25 | 51 |
| 4 | 25 | 25 | 24 | 2.6 | 50 | 25 | 52 |
| 3.4 | 50 | 25 | 24 | 4 | 75 | 25 | 52 |
| 2.6 | 25 | 25 | 24 | 3.4 | 50 | 25 | 56 |
| 9 | 50 | 25 | 25 | 2.6 | 25 | 25 | 57 |
| 5.1 | 25 | 25 | 25 | 5.1 | 75 | 25 | 58 |
| 4 | 50 | 25 | 25 | 4 | 50 | 25 | 58 |
| 2.7 | 25 | 25 | 27 | 4 | 75 | 25 | 60 |
| 5.1 | 25 | 25 | 27 | 9 | 50 | 25 | 61 |
| 4 | 25 | 25 | 28 | 9 | 25 | 25 | 62 |
| 3.4 | 25 | 25 | 28 | 2.7 | 75 | 25 | 65 |
| 2.6 | 25 | 25 | 28 | 2.7 | 25 | 25 | 66 |
| 3.4 | 25 | 25 | 32 | 2.6 | 50 | 25 | 66 |
| 3.4 | 25 | 25 | 32 | 2.6 | 50 | 25 | 68 |
| 5.1 | 50 | 25 | 33 | 3.4 | 25 | 25 | 69 |
| 4 | 50 | 25 | 33 | 9 | 75 | 25 | 71 |
| 5.1 | 25 | 25 | 33 | 2.7 | 25 | 25 | 71 |
| 9 | 25 | 25 | 34 | 5.1 | 75 | 25 | 72 |
| 9 | 75 | 25 | 35 | 3.4 | 25 | 25 | 72 |
| 9 | 25 | 25 | 35 | 2.6 | 75 | 25 | 75 |
| 4 | 25 | 25 | 35 | 5.1 | 75 | 25 | 77 |
| 2.6 | 50 | 25 | 35 | 2.6 | 50 | 25 | 79 |
| 4 | 50 | 25 | 36 | 2.6 | 75 | 25 | 80 |
| 3.4 | 50 | 25 | 36 | 4 | 75 | 25 | 90 |
| 5.1 | 50 | 25 | 36 | 4 | 75 | 25 | 92 |
| 9 | 50 | 25 | 37 | 2.7 | 75 | 25 | 92 |
| | | | | 5.1 | 50 | 25 | 92 |

TABLE 5B
Probability of having LCBV represented
by 5 cm long samples (pLCBV)

| | | | pLCBV = | | | | pLCBV = |
|---|---|---|---|---|---|---|---|
| | | (L/5) − | ((L/5) − | | | (L/5) − | ((L/5) − |
| L | L/5 | 2 | 2)/20 | L | L/5 | 2 | 2)/20 |
| 13 | 2.6 | 0.6 | 0.03 | 39 | 7.8 | 5.8 | 0.29 |
| 15 | 3 | 1 | 0.05 | 40 | 8 | 6 | 0.3 |
| 16 | 3.2 | 1.2 | 0.06 | 44 | 8.8 | 6.8 | 0.34 |
| 18 | 3.6 | 1.6 | 0.08 | 44 | 8.8 | 6.8 | 0.34 |
| 18 | 3.6 | 1.6 | 0.08 | 45 | 9 | 7 | 0.35 |
| 19 | 3.8 | 1.8 | 0.09 | 46 | 9.2 | 7.2 | 0.36 |
| 19 | 3.8 | 1.8 | 0.09 | 49 | 9.8 | 7.8 | 0.39 |
| 23 | 4.6 | 2.6 | 0.13 | 49 | 9.8 | 7.8 | 0.39 |
| 23 | 4.6 | 2.6 | 0.13 | 51 | 10.2 | 8.2 | 0.41 |
| 24 | 4.8 | 2.8 | 0.14 | 52 | 10.4 | 8.4 | 0.42 |
| 24 | 4.8 | 2.8 | 0.14 | 52 | 10.4 | 8.4 | 0.42 |
| 24 | 4.8 | 2.8 | 0.14 | 56 | 11.2 | 9.2 | 0.46 |
| 24 | 4.8 | 2.8 | 0.14 | 57 | 11.4 | 9.4 | 0.47 |
| 25 | 5 | 3 | 0.15 | 58 | 11.6 | 9.6 | 0.48 |
| 25 | 5 | 3 | 0.15 | 58 | 11.6 | 9.6 | 0.48 |
| 25 | 5 | 3 | 0.15 | 60 | 12 | 10 | 0.5 |
| 27 | 5.4 | 3.4 | 0.17 | 61 | 12.2 | 10.2 | 0.51 |
| 27 | 5.4 | 3.4 | 0.17 | 62 | 12.4 | 10.4 | 0.52 |
| 28 | 5.6 | 3.6 | 0.18 | 65 | 13 | 11 | 0.55 |
| 28 | 5.6 | 3.6 | 0.18 | 66 | 13.2 | 11.2 | 0.56 |
| 28 | 5.6 | 3.6 | 0.18 | 66 | 13.2 | 11.2 | 0.56 |
| 32 | 6.4 | 4.4 | 0.22 | 68 | 13.6 | 11.6 | 0.58 |
| 32 | 6.4 | 4.4 | 0.22 | 69 | 13.8 | 11.8 | 0.59 |
| 33 | 6.6 | 4.6 | 0.23 | 71 | 14.2 | 12.2 | 0.61 |
| 33 | 6.6 | 4.6 | 0.23 | 71 | 14.2 | 12.2 | 0.61 |
| 33 | 6.6 | 4.6 | 0.23 | 72 | 14.4 | 12.4 | 0.62 |
| 34 | 6.8 | 4.8 | 0.24 | 72 | 14.4 | 12.4 | 0.62 |
| 35 | 7 | 5 | 0.25 | 75 | 15 | 13 | 0.65 |
| 35 | 7 | 5 | 0.25 | 77 | 15.4 | 13.4 | 0.67 |
| 35 | 7 | 5 | 0.25 | 79 | 15.8 | 13.8 | 0.69 |
| 35 | 7 | 5 | 0.25 | 80 | 16 | 14 | 0.7 |
| 36 | 7.2 | 5.2 | 0.26 | 90 | 18 | 16 | 0.8 |
| 36 | 7.2 | 5.2 | 0.26 | 92 | 18.4 | 16.4 | 0.82 |
| 36 | 7.2 | 5.2 | 0.26 | 92 | 18.4 | 16.4 | 0.82 |
| 37 | 7.4 | 5.4 | 0.27 | 92 | 18.4 | 16.4 | 0.82 |
| 38 | 7.6 | 5.6 | 0.28 | | | | |

TABLE 5C
Probability of having LCBV represented
by 2.5 cm long samples (pLCBV)

| | | | pLCBV = | | | | pLCBV = |
|---|---|---|---|---|---|---|---|
| | | (L/5) − | ((L/5) − | | | (L/5) − | ((L/5) − |
| L | L/2.5 | 2 | 2)/40 | L | L/2.5 | 2 | 2)/40 |
| 13 | 5.2 | 3.2 | 0.08 | 39 | 7.8 | 5.8 | 0.29 |
| 15 | 6 | 4 | 0.1 | 40 | 8 | 6 | 0.3 |
| 16 | 6.4 | 4.4 | 0.11 | 44 | 8.8 | 6.8 | 0.34 |
| 18 | 7.2 | 5.2 | 0.13 | 44 | 8.8 | 6.8 | 0.34 |
| 18 | 7.2 | 5.2 | 0.13 | 45 | 9 | 7 | 0.35 |
| 19 | 7.6 | 5.6 | 0.14 | 46 | 9.2 | 7.2 | 0.36 |
| 19 | 7.6 | 5.6 | 0.14 | 49 | 9.8 | 7.8 | 0.39 |
| 23 | 9.2 | 7.2 | 0.18 | 49 | 9.8 | 7.8 | 0.39 |
| 23 | 9.2 | 7.2 | 0.18 | 51 | 10.2 | 8.2 | 0.41 |
| 24 | 9.6 | 7.6 | 0.19 | 52 | 10.4 | 8.4 | 0.42 |
| 24 | 9.6 | 7.6 | 0.19 | 52 | 10.4 | 8.4 | 0.42 |
| 24 | 9.6 | 7.6 | 0.19 | 56 | 11.2 | 9.2 | 0.46 |
| 24 | 9.6 | 7.6 | 0.19 | 57 | 11.4 | 9.4 | 0.47 |
| 25 | 10 | 8 | 0.2 | 58 | 11.6 | 9.6 | 0.48 |
| 25 | 10 | 8 | 0.2 | 58 | 11.6 | 9.6 | 0.48 |
| 25 | 10 | 8 | 0.2 | 60 | 12 | 10 | 0.5 |
| 27 | 10.8 | 8.8 | 0.22 | 61 | 12.2 | 10.2 | 0.51 |
| 27 | 10.8 | 8.8 | 0.22 | 62 | 12.4 | 10.4 | 0.52 |
| 28 | 11.2 | 9.2 | 0.23 | 65 | 13 | 11 | 0.55 |
| 28 | 11.2 | 9.2 | 0.23 | 66 | 13.2 | 11.2 | 0.56 |
| 28 | 11.2 | 9.2 | 0.23 | 66 | 13.2 | 11.2 | 0.56 |
| 32 | 12.8 | 10.8 | 0.27 | 68 | 13.6 | 11.6 | 0.58 |
| 32 | 12.8 | 10.8 | 0.27 | 69 | 13.8 | 11.8 | 0.59 |
| 33 | 13.2 | 11.2 | 0.28 | 71 | 14.2 | 12.2 | 0.61 |
| 33 | 13.2 | 11.2 | 0.28 | 71 | 14.2 | 12.2 | 0.61 |
| 33 | 13.2 | 11.2 | 0.28 | 72 | 14.4 | 12.4 | 0.62 |
| 34 | 13.6 | 11.6 | 0.29 | 72 | 14.4 | 12.4 | 0.62 |
| 35 | 14 | 12 | 0.3 | 75 | 15 | 13 | 0.65 |
| 35 | 14 | 12 | 0.3 | 77 | 15.4 | 13.4 | 0.67 |
| 35 | 14 | 12 | 0.3 | 79 | 15.8 | 13.8 | 0.69 |
| 35 | 14 | 12 | 0.3 | 80 | 16 | 14 | 0.7 |
| 36 | 14.4 | 12.4 | 0.31 | 90 | 18 | 16 | 0.8 |
| 36 | 14.4 | 12.4 | 0.31 | 92 | 18.4 | 16.4 | 0.82 |
| 36 | 14.4 | 12.4 | 0.31 | 92 | 18.4 | 16.4 | 0.82 |
| 37 | 14.8 | 12.8 | 0.32 | 92 | 18.4 | 16.4 | 0.82 |
| 38 | 15.2 | 13.2 | 0.33 | | | | |

TABLE 5D
Probability of having LCBV represented
by 1 cm long samples (pLCBV)

| | | | pLCBV = | | | | pLCBV = |
|---|---|---|---|---|---|---|---|
| | | (L/5) − | ((L/5) − | | | (L/5) − | ((L/5) − |
| L | L/1 | 2 | 2)/100 | L | L/1 | 2 | 2)/100 |
| 13 | 13 | 11 | 0.11 | 39 | 39 | 37 | 0.37 |
| 15 | 15 | 13 | 0.13 | 40 | 40 | 38 | 0.38 |
| 16 | 16 | 14 | 0.14 | 44 | 44 | 42 | 0.42 |
| 18 | 18 | 16 | 0.16 | 44 | 44 | 42 | 0.42 |
| 18 | 18 | 16 | 0.16 | 45 | 45 | 43 | 0.43 |
| 19 | 19 | 17 | 0.17 | 46 | 46 | 44 | 0.44 |
| 19 | 19 | 17 | 0.17 | 49 | 49 | 47 | 0.47 |
| 23 | 23 | 21 | 0.21 | 49 | 49 | 47 | 0.47 |
| 23 | 23 | 21 | 0.21 | 51 | 51 | 49 | 0.49 |
| 24 | 24 | 22 | 0.22 | 52 | 52 | 50 | 0.5 |
| 24 | 24 | 22 | 0.22 | 52 | 52 | 50 | 0.5 |
| 24 | 24 | 22 | 0.22 | 56 | 56 | 54 | 0.54 |
| 24 | 24 | 22 | 0.22 | 57 | 57 | 55 | 0.55 |
| 25 | 25 | 23 | 0.23 | 58 | 58 | 56 | 0.56 |
| 25 | 25 | 23 | 0.23 | 58 | 58 | 56 | 0.56 |
| 25 | 25 | 23 | 0.23 | 60 | 60 | 58 | 0.58 |
| 27 | 27 | 25 | 0.25 | 61 | 61 | 59 | 0.59 |
| 27 | 27 | 25 | 0.25 | 62 | 62 | 60 | 0.6 |
| 28 | 28 | 26 | 0.26 | 65 | 65 | 63 | 0.63 |
| 28 | 28 | 26 | 0.26 | 66 | 66 | 64 | 0.64 |
| 28 | 28 | 26 | 0.26 | 66 | 66 | 64 | 0.64 |
| 32 | 32 | 30 | 0.3 | 68 | 68 | 66 | 0.66 |
| 32 | 32 | 30 | 0.3 | 69 | 69 | 67 | 0.67 |
| 33 | 33 | 31 | 0.31 | 71 | 71 | 69 | 0.69 |
| 33 | 33 | 31 | 0.31 | 71 | 71 | 69 | 0.69 |
| 33 | 33 | 31 | 0.31 | 72 | 72 | 70 | 0.7 |
| 34 | 34 | 32 | 0.32 | 72 | 72 | 70 | 0.7 |
| 35 | 35 | 33 | 0.33 | 75 | 75 | 73 | 0.73 |
| 35 | 35 | 33 | 0.33 | 77 | 77 | 75 | 0.75 |
| 35 | 35 | 33 | 0.33 | 79 | 79 | 77 | 0.77 |
| 35 | 35 | 33 | 0.33 | 80 | 80 | 78 | 0.78 |
| 36 | 36 | 34 | 0.34 | 90 | 90 | 88 | 0.88 |
| 36 | 36 | 34 | 0.34 | 92 | 92 | 90 | 0.9 |
| 36 | 36 | 34 | 0.34 | 92 | 92 | 90 | 0.9 |
| 37 | 37 | 35 | 0.35 | 92 | 92 | 90 | 0.9 |
| 38 | 38 | 36 | 0.36 | | | | |

TABLE 6A
Data pertaining to MPS models with cross section of 100 cm²

| BS | BP | SCS | Length (L) cm |
|---|---|---|---|
| 9 | 25 | 100 | 16 |
| 9 | 25 | 100 | 19 |
| 5.1 | 25 | 100 | 24 |
| 9 | 50 | 100 | 25 |
| 5.1 | 25 | 100 | 26 |
| 5.1 | 50 | 100 | 32 |
| 9 | 25 | 100 | 34 |
| 5.1 | 50 | 100 | 34 |
| 9 | 25 | 100 | 36 |
| 4 | 25 | 100 | 37 |
| 5.1 | 50 | 100 | 42 |
| 4 | 25 | 100 | 42 |
| 5.1 | 25 | 100 | 43 |
| 4 | 25 | 100 | 47 |
| 5.1 | 25 | 100 | 48 |
| 4 | 25 | 100 | 49 |
| 3.4 | 25 | 100 | 49 |
| 3.4 | 25 | 100 | 49 |
| 2.7 | 25 | 100 | 50 |
| 5.1 | 50 | 100 | 53 |
| 9 | 25 | 100 | 54 |
| 4 | 50 | 100 | 60 |
| 4 | 50 | 100 | 60 |
| 5.1 | 25 | 100 | 64 |
| 4 | 50 | 100 | 64 |
| 3.4 | 25 | 100 | 64 |
| 2.7 | 50 | 100 | 64 |
| 2.6 | 25 | 100 | 67 |
| 9 | 50 | 100 | 72 |
| 9 | 50 | 100 | 73 |
| 4 | 50 | 100 | 74 |
| 2.7 | 50 | 100 | 76 |
| 2.7 | 25 | 100 | 80 |
| 2.6 | 25 | 100 | 80 |
| 5.1 | 50 | 100 | 84 |
| 5.1 | 75 | 100 | 84 |
| 9 | 50 | 100 | 85 |
| 9 | 50 | 100 | 90 |
| 2.6 | 25 | 100 | 91 |
| 3.4 | 25 | 100 | 96 |
| 2.7 | 25 | 100 | 96 |
| 2.7 | 25 | 100 | 98 |

TABLE 6B
Probability of having LCBV represented
by 10 cm long samples (pLCBV)

| L | L/5 | (L/5) − 2 | pLCBV = ((L/5) − 2)/10 |
|---|---|---|---|
| 16 | 1.6 | −0.4 | −0.04 |
| 19 | 1.9 | −0.1 | −0.01 |
| 24 | 2.4 | 0.4 | 0.04 |
| 25 | 2.5 | 0.5 | 0.05 |
| 26 | 2.6 | 0.6 | 0.06 |
| 32 | 3.2 | 1.2 | 0.12 |
| 34 | 3.4 | 1.4 | 0.14 |
| 34 | 3.4 | 1.4 | 0.14 |
| 36 | 3.6 | 1.6 | 0.16 |
| 37 | 3.7 | 1.7 | 0.17 |
| 42 | 4.2 | 2.2 | 0.22 |
| 42 | 4.2 | 2.2 | 0.22 |
| 43 | 4.3 | 2.3 | 0.23 |
| 47 | 4.7 | 2.7 | 0.27 |
| 48 | 4.8 | 2.8 | 0.28 |
| 49 | 4.9 | 2.9 | 0.29 |
| 49 | 4.9 | 2.9 | 0.29 |
| 49 | 4.9 | 2.9 | 0.29 |
| 50 | 5 | 3 | 0.3 |
| 53 | 5.3 | 3.3 | 0.33 |
| 54 | 5.4 | 3.4 | 0.34 |
| 60 | 6 | 4 | 0.4 |
| 60 | 6 | 4 | 0.4 |
| 64 | 6.4 | 4.4 | 0.44 |
| 64 | 6.4 | 4.4 | 0.44 |
| 64 | 6.4 | 4.4 | 0.44 |
| 64 | 6.4 | 4.4 | 0.44 |
| 67 | 6.7 | 4.7 | 0.47 |
| 72 | 7.2 | 5.2 | 0.52 |
| 73 | 7.3 | 5.3 | 0.53 |
| 74 | 7.4 | 5.4 | 0.54 |
| 76 | 7.6 | 5.6 | 0.56 |
| 80 | 8 | 6 | 0.6 |
| 80 | 8 | 6 | 0.6 |
| 84 | 8.4 | 6.4 | 0.64 |
| 84 | 8.4 | 6.4 | 0.64 |
| 85 | 8.5 | 6.5 | 0.65 |
| 90 | 9 | 7 | 0.7 |
| 91 | 9.1 | 7.1 | 0.71 |
| 96 | 9.6 | 7.6 | 0.76 |
| 96 | 9.6 | 7.6 | 0.76 |
| 98 | 9.8 | 7.8 | 0.78 |

TABLE 6C
Probability of having LCBV represented
by 20 cm long samples (pLCBV)

| L | L/2.5 | (L/5) − 2 | pLCBV = ((L/5) − 2)/5 |
|---|---|---|---|
| 16 | 0.8 | −1.2 | −0.24 |
| 19 | 0.95 | −1.05 | −0.21 |
| 24 | 1.2 | −0.8 | −0.16 |
| 25 | 1.25 | −0.75 | −0.15 |
| 26 | 1.3 | −0.7 | −0.14 |
| 32 | 1.6 | −0.4 | −0.08 |
| 34 | 1.7 | −0.3 | −0.06 |
| 34 | 1.7 | −0.3 | −0.06 |
| 36 | 1.8 | −0.2 | −0.04 |
| 37 | 1.85 | −0.15 | −0.03 |
| 42 | 2.1 | 0.1 | 0.02 |
| 42 | 2.1 | 0.1 | 0.02 |
| 43 | 2.15 | 0.15 | 0.03 |
| 47 | 2.35 | 0.35 | 0.07 |
| 48 | 2.4 | 0.4 | 0.08 |
| 49 | 2.45 | 0.45 | 0.09 |
| 49 | 2.45 | 0.45 | 0.09 |
| 49 | 2.45 | 0.45 | 0.09 |
| 50 | 2.5 | 0.5 | 0.1 |
| 53 | 2.65 | 0.65 | 0.13 |
| 54 | 2.7 | 0.7 | 0.14 |
| 60 | 3 | 1 | 0.2 |
| 60 | 3 | 1 | 0.2 |
| 64 | 3.2 | 1.2 | 0.24 |
| 64 | 3.2 | 1.2 | 0.24 |
| 64 | 3.2 | 1.2 | 0.24 |
| 64 | 3.2 | 1.2 | 0.24 |
| 67 | 3.35 | 1.35 | 0.27 |
| 72 | 3.6 | 1.6 | 0.32 |
| 73 | 3.65 | 1.65 | 0.33 |
| 74 | 3.7 | 1.7 | 0.34 |
| 76 | 3.8 | 1.8 | 0.36 |
| 80 | 4 | 2 | 0.4 |
| 80 | 4 | 2 | 0.4 |
| 84 | 4.2 | 2.2 | 0.44 |
| 84 | 4.2 | 2.2 | 0.44 |
| 85 | 4.25 | 2.25 | 0.45 |
| 90 | 4.5 | 2.5 | 0.5 |
| 91 | 4.55 | 2.55 | 0.51 |
| 96 | 4.8 | 2.8 | 0.56 |
| 96 | 4.8 | 2.8 | 0.56 |
| 98 | 4.9 | 2.9 | 0.58 |

TABLE 6D
Probability of having LCBV represented
by 25 cm long samples (pLCBV)

| L | L/1 | (L/5) − 2 | pLCBV = ((L/5) − 2)/4 |
|---|---|---|---|
| 16 | 0.64 | −1.36 | −0.34 |
| 19 | 0.76 | −1.24 | −0.31 |
| 24 | 0.96 | −1.04 | −0.26 |
| 25 | 1 | −1 | −0.25 |
| 26 | 1.04 | −0.96 | −0.24 |
| 32 | 1.28 | −0.72 | −0.18 |
| 34 | 1.36 | −0.64 | −0.16 |
| 34 | 1.36 | −0.64 | −0.16 |
| 36 | 1.44 | −0.56 | −0.14 |
| 37 | 1.48 | −0.52 | −0.13 |
| 42 | 1.68 | −0.32 | −0.08 |
| 42 | 1.68 | −0.32 | −0.08 |
| 43 | 1.72 | −0.28 | −0.07 |
| 47 | 1.88 | −0.12 | −0.03 |
| 48 | 1.92 | −0.08 | −0.02 |
| 49 | 1.96 | −0.04 | −0.01 |
| 49 | 1.96 | −0.04 | −0.01 |
| 49 | 1.96 | −0.04 | −0.01 |
| 50 | 2 | 0 | 0 |
| 53 | 2.12 | 0.12 | 0.03 |
| 54 | 2.16 | 0.16 | 0.04 |
| 60 | 2.4 | 0.4 | 0.1 |
| 60 | 2.4 | 0.4 | 0.1 |
| 64 | 2.56 | 0.56 | 0.14 |
| 64 | 2.56 | 0.56 | 0.14 |
| 64 | 2.56 | 0.56 | 0.14 |
| 64 | 2.56 | 0.56 | 0.14 |
| 67 | 2.68 | 0.68 | 0.17 |
| 72 | 2.88 | 0.88 | 0.22 |
| 73 | 2.92 | 0.92 | 0.23 |
| 74 | 2.96 | 0.96 | 0.24 |
| 76 | 3.04 | 1.04 | 0.26 |
| 80 | 3.2 | 1.2 | 0.3 |
| 80 | 3.2 | 1.2 | 0.3 |
| 84 | 3.36 | 1.36 | 0.34 |
| 84 | 3.36 | 1.36 | 0.34 |
| 85 | 3.4 | 1.4 | 0.35 |
| 90 | 3.6 | 1.6 | 0.4 |
| 91 | 3.64 | 1.64 | 0.41 |
| 96 | 3.84 | 1.84 | 0.46 |
| 96 | 3.84 | 1.84 | 0.46 |
| 98 | 3.92 | 1.92 | 0.48 |

### Example 12: Application of the Above Described Model

The results highlighted the importance of BP, BS and SCS as variables that control the representation of the Thalassinoides connectivity in samples, as shown in FIGS. 7A-7F, FIGS. 8A-8C, and FIGS. 9A-9C. Further, it may be quantified from the above stated examples that BP, BS and SCS quantitatively control representation of Thalassinoides connectivity when sampling. The system 100 and the method 200 provided in the present disclosure are vital to determine the correct dimension of samples that can represent Thalassinoides connectivity and acquiring permeability measurements to represent a reservoir volume containing Thalassinoides. The logistic regression model developed in the present disclosure provides a method to collectively link BP, BS and SCS as controls on Thalassinoides connectivity across a 1-meter-long sample. Furthermore, the weight of each of these variables as controllers on the Thalassinoides connectivity is calculated by the logistic regression model, as shown in Tables 3A-3I. An important product of the logistic regression model is the equation of the probability of representing Thalassinoides connectivity in 1-meter-long digital samples. Where the logistic regression produces a high probability for an SCS to show Thalassinoides connectivity across that 1 m sample, a shorter segment of that same 1 m sample may show the same connectivity. Thus, the equation provided by the present disclosure can be used as a lower limit on the SCS required to represent Thalassinoides permeability. In one implementation of the present disclosure, to measure permeability on a sample that is shorter than 1 m, the BP and BS of that sample may be fed as input to the system 100 and further calculate the SCS having the desired high probability of connecting across the 1 m sample, an example is documented in Table 7. In addition, the output SCS may be used for sampling and measuring. In another implementation of the present disclosure, the Thalassinoides-bearing strata in the Hanifa formation in central Saudi Arabia was examined. Thalassinoides of the Hanifa formation were examined and burrow attributes, including BP (range from 10% to 40%) and BS (shaft diameters range from 0.86 cm to 3.6 cm with mean of 1.5 cm), were documented. A particularly large diameter core (drilled with a 10-inch bit) was studied to characterize Thalassinoides using CT-scans. The core was characterized in its entirety and evaluated to examine the scale of subsamples that would produce connectivity across the entire 26.5 cm length of the core. The CT scan of the full sample showed a BP of 38%. The Thalassinoides in that large core connected across the top and bottom of the core. In one case, if 1.5 cm BS and 38% BP is provided as input to the logistic regression equation developed herein, it produces a probability curve of Thalassinoides connecting across a 1 m-long sample given various sample cross sections, as shown in FIG. 10. Probabilities greater than 0.5 require sample cross sections of 64 cm² (8 cm side length) and higher, as shown in FIG. 10. By digitally subsampling the scanned 26.5 cm-long core, it was found that prisms with cross sections of 51.84 cm² (7.2 cm side length) showed 100% probability of Thalassinoides connecting across the top and bottom, and samples with cross sections of about 30 cm² (5.5 cm side length) showed 50% probability of Thalassinoides connecting. The SCS output from the 1-m logistic regression equation is slightly larger than the SCS required for Thalassinoides connectivity in the 26.5 cm example. This supports the results from the logistic regression equation, and can be used to guide the minimum SCS of samples that are shorter that 1 m, sample lengths within the realm of easy analysis by standard techniques.

TABLE 7
Probability of Connectivity (PC) of 1-meter-long columnar sample

| PC | BS (cm) | PB (%) | SCS (cm²) |
|---|---|---|---|
| 0.130 | 1.5 | 25 | 1 |
| 0.134 | 1.5 | 25 | 4 |
| 0.139 | 1.5 | 25 | 9 |
| 0.148 | 1.5 | 25 | 16 |
| 0.159 | 1.5 | 25 | 25 |
| 0.174 | 1.5 | 25 | 36 |
| 0.193 | 1.5 | 25 | 49 |
| 0.216 | 1.5 | 25 | 64 |
| 0.245 | 1.5 | 25 | 81 |
| 0.281 | 1.5 | 25 | 100 |
| 0.323 | 1.5 | 25 | 121 |
| 0.374 | 1.5 | 25 | 144 |
| 0.432 | 1.5 | 25 | 169 |
| 0.496 | 1.5 | 25 | 196 |
| 0.566 | 1.5 | 25 | 225 |
| 0.638 | 1.5 | 25 | 256 |
| 0.708 | 1.5 | 25 | 289 |
| 0.772 | 1.5 | 25 | 324 |
| 0.829 | 1.5 | 25 | 361 |
| 0.876 | 1.5 | 25 | 400 |
| 0.913 | 1.5 | 25 | 441 |
| 0.941 | 1.5 | 25 | 484 |
| 0.961 | 1.5 | 25 | 529 |
| 0.975 | 1.5 | 25 | 576 |
| 0.984 | 1.5 | 25 | 625 |
| 0.990 | 1.5 | 25 | 676 |
| 0.994 | 1.5 | 25 | 729 |
| 0.997 | 1.5 | 25 | 784 |
| 0.998 | 1.5 | 25 | 841 |
| 0.999 | 1.5 | 25 | 900 |

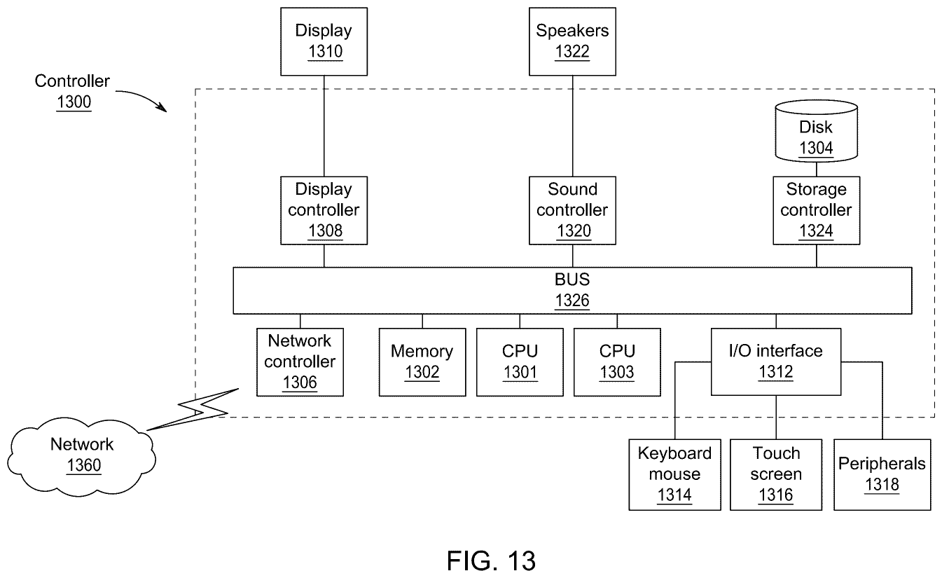

Next, further details of the hardware description of the computing environment according to exemplary embodiments is described with reference to FIG. 13. In FIG. 13, a controller 1300 is described is representative of the system 100 of FIG. 1 in which the controller is a computing device which includes a CPU 1301 which performs the processes described above/below. The process data and instructions may be stored in memory 1302. These processes and instructions may also be stored on a storage medium disk 1304 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 1301, 1303 and an operating system such as Microsoft Windows 7, Microsoft Windows 10, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, CPU 1301 or CPU 1303 may be a Xenon or Core processor from Intel of America or an Opteron processor from AMD of America, or may be other processor types that would be recognized by one of ordinary skill in the art. Alternatively, the CPU 1301, 1303 may be implemented on an FPGA, ASIC, PLD or using discrete logic circuits, as one of ordinary skill in the art would recognize. Further, CPU 1301, 1303 may be implemented as multiple processors cooperatively working in parallel to perform the instructions of the inventive processes described above.

The computing device in FIG. 13 also includes a network controller 1306, such as an Intel Ethernet PRO network interface card from Intel Corporation of America, for interfacing with network 1360. As can be appreciated, the network 1360 can be a public network, such as the Internet, or a private network such as an LAN or WAN network, or any combination thereof and can also include PSTN or ISDN sub-networks. The network 1360 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The computing device further includes a display controller 1308, such as a NVIDIA GeForce GTX or Quadro graphics adaptor from NVIDIA Corporation of America for interfacing with display 1310, such as a Hewlett Packard HPL2445w LCD monitor. A general purpose I/O interface 1312 interfaces with a keyboard and/or mouse 1314 as well as a touch screen panel 1316 on or separate from display 1310. General purpose I/O interface also connects to a variety of peripherals 1318 including printers and scanners, such as an OfficeJet or DeskJet from Hewlett Packard.

A sound controller 1320 is also provided in the computing device such as Sound Blaster X-Fi Titanium from Creative, to interface with speakers/microphone 1322 thereby providing sounds and/or music.

The general-purpose storage controller 1324 connects the storage medium disk 1304 with communication bus 1326, which may be an ISA, EISA, VESA, PCI, or similar, for interconnecting all of the components of the computing device. A description of the general features and functionality of the display 1310, keyboard and/or mouse 1314, as well as the display controller 1308, storage controller 1324, network controller 1306, sound controller 1320, and general purpose I/O interface 1312 is omitted herein for brevity as these features are known.

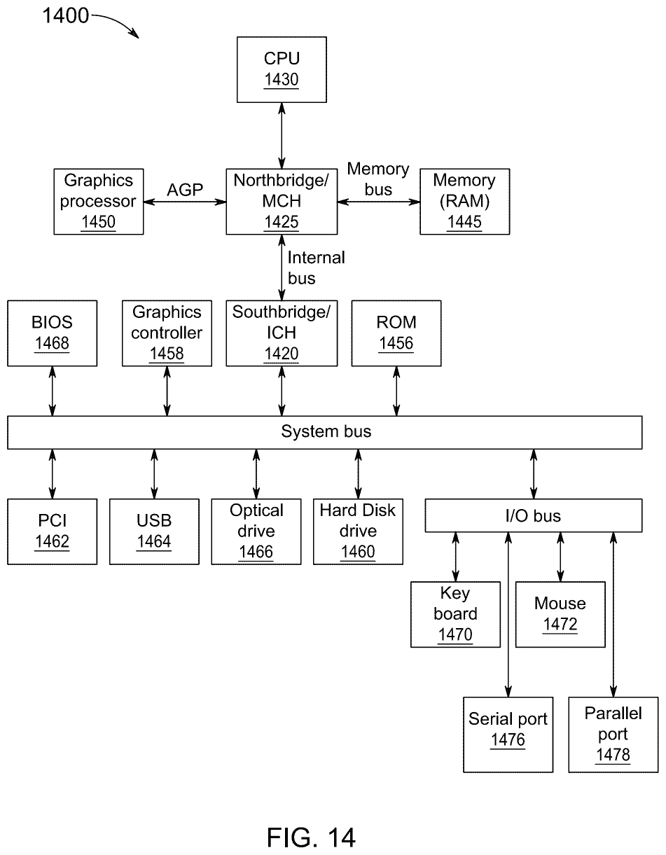

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 14.

FIG. 14 shows a schematic diagram of a data processing system, according to certain embodiments, for performing the functions of the exemplary embodiments. The data processing system is an example of a computer in which code or instructions implementing the processes of the illustrative embodiments may be located.

In FIG. 4, data processing system 1400 employs a hub architecture including a north bridge and memory controller hub (NB/MCH) 1425 and a south bridge and input/output (I/O) controller hub (SB/ICH) 1420. The central processing unit (CPU) 1430 is connected to NB/MCH 1425. The NB/MCH 1425 also connects to the memory 1445 via a memory bus and connects to the graphics processor 1450 via an accelerated graphics port (AGP). The NB/MCH 1425 also connects to the SB/ICH 1420 via an internal bus (e.g., a unified media interface or a direct media interface). The CPU Processing unit 1430 may contain one or more processors and even may be implemented using one or more heterogeneous processor systems.

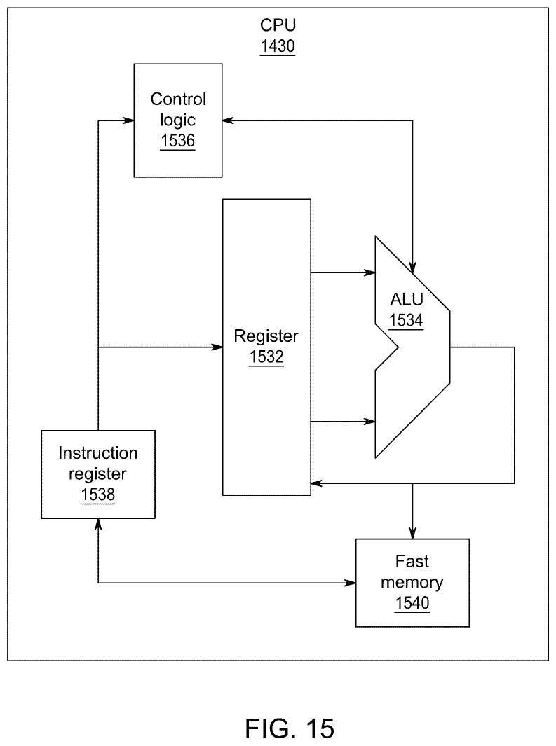

For example, FIG. 15 shows one implementation of CPU 1430. In one implementation, the instruction registers 1538 retrieves instructions from the fast memory 1540. At least part of these instructions is fetched from the instruction register 1538 by the control logic 1536 and interpreted according to the instruction set architecture of the CPU 1430. Part of the instructions can also be directed to the register 1532. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 1534 that loads values from the register 1532 and performs logical and mathematical operations on the loaded values according to the instructions. The results from these operations can be feedback into the register and/or stored in the fast memory 1540. According to certain implementations, the instruction set architecture of the CPU 1430 can use a reduced instruction set architecture, a complex instruction set architecture, a vector processor architecture, a very large instruction word architecture. Furthermore, the CPU 1430 can be based on the Von Neuman model or the Harvard model. The CPU 1430 can be a digital signal processor, an FPGA, an ASIC, a PLA, a PLD, or a CPLD. Further, the CPU 1430 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 14, the data processing system 1400 can include that the SB/ICH 1420 is coupled through a system bus to an I/O Bus, a read only memory (ROM) 1456, universal serial bus (USB) port 1464, a flash binary input/output system (BIOS) 1468, and a graphics controller 1458. PCI/PCIe devices can also be coupled to SB/ICH 1488 through a PCI bus 1462.

The PCI devices may include, for example, Ethernet adapters, add-in cards, and PC cards for notebook computers. The Hard disk drive 1460 and CD-ROM 1466 can use, for example, an integrated drive electronics (IDE) or serial advanced technology attachment (SATA) interface. In one implementation the I/O bus can include a super I/O (SIO) device.

Further, the hard disk drive (HDD) 1460 and optical drive 1466 can also be coupled to the SB/ICH 1420 through a system bus. In one implementation, a keyboard 1470, a mouse 1472, a parallel port 1478, and a serial port 1476 can be connected to the system bus through the I/O bus. Other peripherals and devices that can be connected to the SB/ICH 1420 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

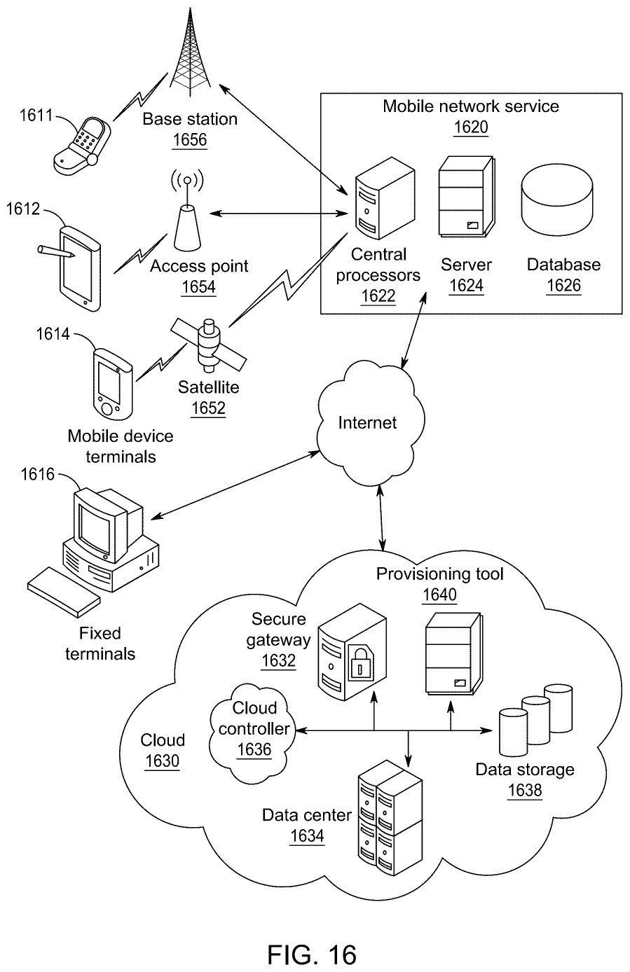

The functions and features described herein may also be executed by various distributed components of a system. For example, one or more processors may execute these system functions, wherein the processors are distributed across multiple components communicating in a network. The distributed components may include one or more client and server machines, such as cloud 1630 including a cloud controller 1636, a secure gateway 1632, a data centre 1634, data storage 1638 and a provisioning tool 1640, and mobile network services 1620 including central processors 1622, a server 1624 and a database 1626, which may share processing, as shown by FIG. 16, in addition to various human interface and communication devices (e.g., display monitors 1616, smart phones 1610, tablets 1612, personal digital assistants (PDAs) 1614). The network may be a private network, such as a LAN, satellite 1652 or WAN 1654, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A method of estimating a permeability of a bioturbated reservoir based on a Thalassinoides connectivity, comprising:
generating a plurality of geocellular models from a Thalassinoides morphology;
converting the plurality of geocellular models to a plurality of training images each having a host rock matrix and Thalassinoides burrows;
measuring a plurality of statistical parameters from the plurality of training images to obtain a width of a Thalassinoides shaft;
creating a plurality of samples each having a burrow percentage, a burrow size, and a sample cross section from the plurality of geocellular models;
determining a largest connected burrow volume (LCBV) of each sample of the plurality of samples based on the Thalassinoides burrows to obtain a burrow connectivity; and
computing the Thalassinoides connectivity based on the burrow connectivity to thereby estimate the permeability of the bioturbated reservoir.

2. The method of claim 1, wherein each geocellular model of the plurality of geocellular models comprises a three-dimensional multipoint statistics (3DMPS) model having a three-dimensional volume of about 1 m³.

3. The method of claim 2, wherein the plurality of geocellular models includes 18 3DMPS models.

4. The method of claim 3, wherein each 3DMPS model of the 18 3DMPS models is constructed based on an Eltom method.

5. The method of claim 4, wherein the creating further comprises:
extracting a plurality of columnar samples from each geocellular model of the plurality of geocellular models;
extracting a plurality of subsamples each having a column cross section from each columnar sample of the plurality of columnar samples, wherein an area of the column cross section is between 25 cm² and 900 cm²; and
combining the plurality of subsamples to obtain the plurality of samples.

6. The method of claim 5, wherein the plurality of subsamples includes 6 subsamples and wherein the area of the column cross section of the plurality of subsamples is 25 cm², 100 cm², 225 cm², 400 cm², 625 cm², or 900 cm².

7. The method of claim 1, wherein the burrow percentage of each sample of the plurality of samples is selected from the group consisting of 20%, 50%, and 75%.

8. The method of claim 1, wherein the burrow size of each sample of the plurality of samples is between 2.6 cm and 9 cm.

9. The method of claim 1, wherein the determining further comprises:
determining the LCBV of each sample of the plurality of samples based on an Eltom method;
determining whether the LCBV of each sample of the plurality of samples is connected across from a top to a bottom of each sample of the plurality of samples;
indicating, when the LCBV is connected across from the top to the bottom, the LCBV as a connected burrow; and
measuring a length and a position of the LCBV to determine the burrow connectivity.

10. The method of claim 9, wherein the computing further comprises:
dividing the plurality of samples into a training set and a validation set;
running a logistic regression analysis with the training set and the burrow connectivity to obtain a logistic regression result;
validating the logistic regression result with the validation set and the burrow connectivity to obtain a probability equation; and
computing the Thalassinoides connectivity based on the probability equation.

11. A system for estimating a permeability of bioturbated reservoirs represented by a Thalassinoides connectivity, comprising:
a processor configured to execute a program instruction;
a memory having the program instruction, wherein the memory is connected to the processor;
an input device connected to the processor and configured to receive a plurality of computed tomography (CT) scan images each having a Thalassinoides morphology; and
a display device configured to display the Thalassinoides connectivity,
wherein the program instruction comprises:
generating a plurality of geocellular models from the Thalassinoides morphology of the plurality of CT scan images;
converting the plurality of geocellular models to a plurality of training images each having a host rock matrix and Thalassinoides burrows;
measuring a plurality of statistical parameters from the plurality of training images to obtain a width of a Thalassinoides shaft;
creating a plurality of samples each having a burrow percentage, a burrow size, and a sample cross section from the plurality of geocellular models;
determining a largest connected burrow volume (LCBV) of each sample of the plurality of samples based on the Thalassinoides burrows to obtain a burrow connectivity; and
computing the Thalassinoides connectivity based on the burrow connectivity to thereby estimate the permeability of bioturbated reservoirs.

12. The system of claim 11, wherein each geocellular model of the plurality of geocellular models comprises a three-dimensional multipoint statistics (3DMPS) model having a three-dimensional volume of about 1 m³.

13. The system of claim 12, wherein the plurality of geocellular models includes 18 3DMPS models.

14. The system of claim 13, wherein each 3DMPS model of the 18 3DMPS models is constructed based on an Eltom method.

15. The system of claim 14, wherein the creating further comprises:
extracting a plurality of columnar samples from each geocellular model of the plurality of geocellular models;
extracting a plurality of subsamples each having a column cross section from each columnar sample of the plurality of columnar samples, wherein an area of the column cross section is between 25 cm² and 900 cm²; and
combining the plurality of subsamples to obtain the plurality of samples.

16. The system of claim 15, wherein the plurality of subsample includes 6 subsamples and wherein the area of the column cross section of the plurality of subsamples is 25 cm², 100 cm², 225 cm², 400 cm², 625 cm², or 900 cm².

17. The system of claim 11, wherein the burrow percentage of each sample of the plurality of samples is selected from the group consisting of 20%, 50%, and 75%.

18. The system of claim 11, wherein the burrow size of each sample of the plurality of samples is between 2.6 cm and 9 cm.

19. The system of claim 11, wherein the determining further comprises:
determining the LCBV of each sample of the plurality of samples based on an Eltom method;
determining whether the LCBV of each sample of the plurality of samples is connected across from a top to a bottom of each sample of the plurality of samples;
indicating, when the LCBV is connected across from the top to the bottom, the LCBV as a connected burrow; and
measuring a length and a position of the LCBV to determine the burrow connectivity.

20. The system of claim 19, wherein the computing further comprises:
dividing the plurality of samples into a training set and a validation set;
running a logistic regression analysis with the training set and the burrow connectivity to obtain a logistic regression result;
validating the logistic regression result with the validation set and the burrow connectivity to obtain a probability equation; and
computing the Thalassinoides connectivity based on the probability equation.
